(19) United States
(12) Patent Application Publication
(10) Pub. No.: US 2025/0387422 A1
(43) Pub. Date: Dec. 25, 2025

(54) FORMULATION OF AN AMPHOTERICIN B HYBRID AMIDE DERIVATIVE IN DSGPEG2K MICELLES

(71) Applicants: The Board of Trustees of the University of Illinois, Urbana, IL (US); Elion Therapeutics, Inc., New York, NY (US)

(72) Inventors: Martin D. Burke, Champaign, IL (US); Arun Maji, Champaign, IL (US); Jeffry G. Weers, Half Moon Bay, CA (US)

(21) Appl. No.: 18/877,792

(22) PCT Filed: Jun. 26, 2023
(86) PCT No.: PCT/US23/26220

§ 371 Date: Dec. 20, 2024

Related U.S. Application Data
(60) Provisional application No. 63/355,345, filed on Jun. 24, 2022.

(51) Int. Cl. A61K 31/7048; A61K 9/00; A61K 47/14

(52) U.S. Cl. CPC A61K 31/7048; A61K 9/0019; A61K 47/14

(57) ABSTRACT

Disclosed are compositions comprising a lipid polymer excipient and AmB or an AmB derivative. The lipid polymer excipient can form micelles when formulated with AmB or the AmB derivative and can solubilize and stabilize the drug. Also disclosed are methods of treating a fungal infection using the compositions.

### RELATED APPLICATIONS

The application claims the benefit of priority to U.S. Provisional Patent Application Ser. No. 63/355,345, filed Jun. 24, 2022.

### FIELD

The present disclosure provides compositions of amphotericin B and derivatives thereof having improved solution stability and plasma compatibility, and methods of using such formulations. More particularly, the present disclosure relates to micellar formulations comprising amphotericin B or a derivative thereof and a block copolymer that not only stabilize the active pharmaceutical ingredient, but also unexpectedly improve the potency and increase the half-life of the compound.

### BACKGROUND

Morbidity and mortality from invasive fungal infections are significant, and largely caused by two genera of fungal pathogens: Candida and Aspergillus. Candida species are the 4th most common pathogen isolated in all bloodstream infections. Treatment for invasive candidiasis has a limited success rate (50-70%), and this is typically only in the healthiest patients. Attributable mortality for invasive candidiasis is substantial (20-30%). The incidence of invasive aspergillosis due to A. fumigatus has increased three-fold in the last decade and its mortality has risen by over 300%. Moreover, current therapy for invasive aspergillosis has a lower 40-50% treatment success rate. Invasive aspergillosis is consistently a leading killer in immunocompromised patients, and invasive mold infections (fusariosis, scedosporosis, and mucromycosis) have even higher mortality rates and no effective therapeutic options. The current guideline-recommended first line therapeutic for invasive aspergillosis, as well as most other invasive mold infections, is the triazole antifungal voriconazole. However, pan-triazole resistance in Aspergillus is as high as 30% in some locations and amongst certain high-risk patient groups. Recognizing this lack of effective treatments, the Infectious Diseases Society of America highlighted A. fumigatus as one of only six pathogens where a “substantive breakthrough is urgently needed.”

Amphotericin B (AmB) is an exceptionally promising starting point, because this drug has potent and dose-dependent fungicidal activity against a broad range of fungal pathogens and has evaded resistance for over half a century. The fungicidal, as opposed to fungistatic, activity of AmB is essential in immunocompromised patients who lack a robust immune system to help clear an infection. Broad antifungal activity is especially important in critically ill patients when the identity of the pathogen is unknown and immediate empirical therapy is required. An international expert panel recently mandated that novel therapeutic approaches centered around AmB, with no resistance issues, are required. The problem is that AmB is exceptionally toxic, which limits its use to low-dose protocols that often fail to eradicate disease.

A new, paradigm-shifting mechanistic understanding of AmB that evaded the field for half a century was achieved. Previous studies report AmB binding to sterols, which was thought to primarily drive formation of membrane-permeabilizing pores to kill both fungal and human cells. After 10 years of intensive synthesis-enabled atomistic interrogations of this natural product and frontier SSNMR experiments, it is discovered that AmB primarily kills both fungal and human cells by forming a cytocidal extramembranous sterol sponge. This large aggregate sits on the surface of lipid bilayers and rapidly extracts membrane sterols, which leads to cell death. Membrane permeabilization is not required. Based on this new mechanism and increasingly refined structural information, it is proposed that a small molecule-based ligand-selective allosteric effect could enable selective binding of ergosterol over cholesterol. Guided by this model, a new derivative, C2′epiAmB was found to eliminate cholesterol binding and thus mammalian toxicity.

A limitation with C2′epiAmB, however, is lack of potency against a number of clinically relevant yeast and molds. Other AmB derivatives suffer from poor plasma compatibility and solution stability. Thus, there remains a need to develop formulations of AmB derivatives that retain potent, broad spectrum, and resistance-evasive fungicidal activity, minimize dose-limiting toxicities, and improve the plasma compatibility and solution stability of these important compounds.

### SUMMARY OF THE INVENTION

In certain aspects, the present invention provides a composition, comprising:
(i) a lipid polymer excipient having the structure of formula (X);

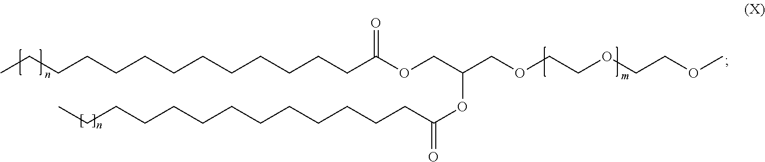

wherein each occurrence of n is independently selected from 0-10; and
m is selected from 10-60; and
(ii) a compound, or a pharmaceutically acceptable salt thereof, selected from the group consisting of:

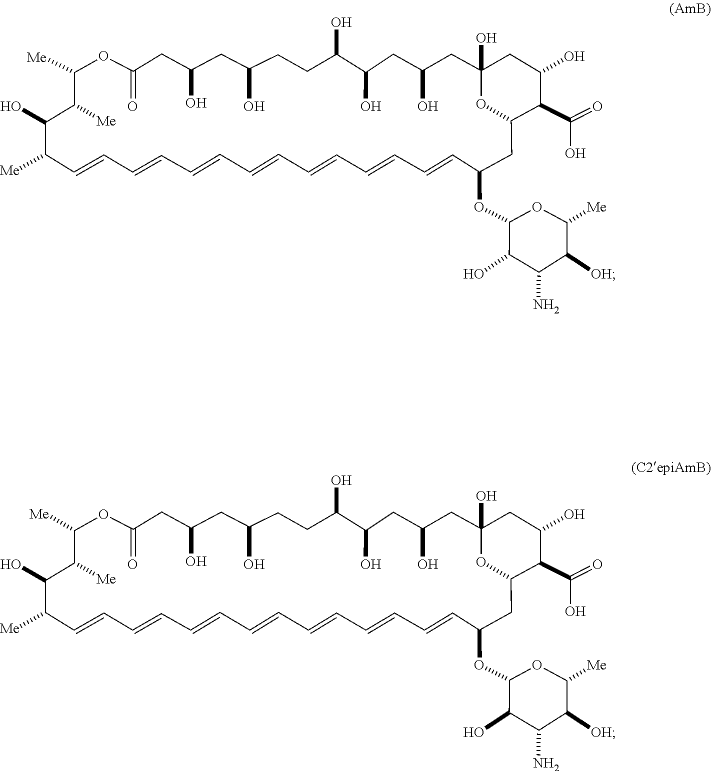

a compound having the structure of formula (I):

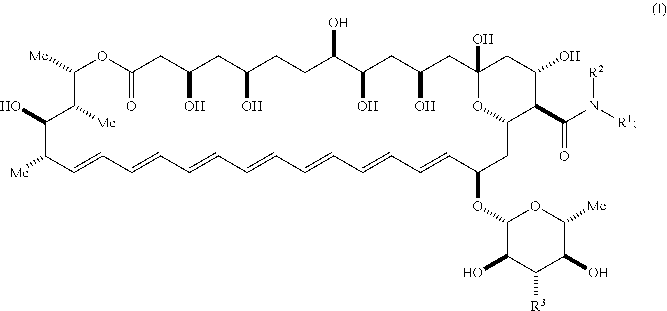

and
a compound having the structure of formula (II):

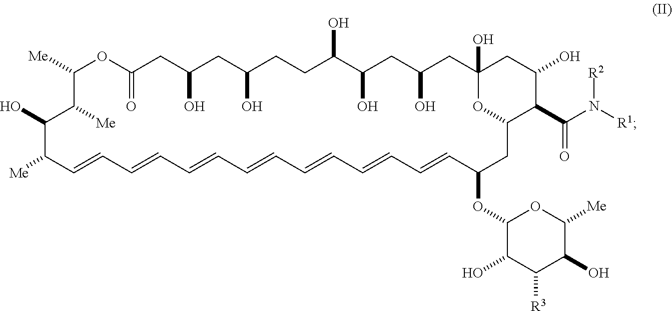

wherein:
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², taken together with the nitrogen to which they are attached, form a substituted or unsubstituted 3- to 10-membered heterocyclyl;
R³ is —NR⁵R⁶, substituted or unsubstituted amino, substituted or unsubstituted urea, substituted or unsubstituted carbamate or substituted or unsubstituted guanidinyl;
R⁴ is hydrogen or substituted or unsubstituted C₁₋₆ alkyl;
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, taken together with the nitrogen to which they are attached, form a substituted or unsubstituted 3- to 10-membered heterocyclyl; and R^{f} is selected from the group consisting of 2-alken-1-yl, tert-butyl, benzyl and fluorenylmethyl.

In certain embodiments, the compound is:

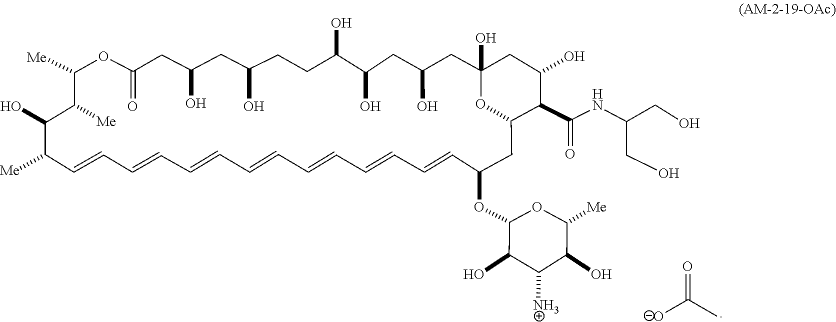

Also provided herein are methods of treating a fungal infection comprising administering to a subject in need thereof a therapeutically effective amount of a composition of the invention.

In another aspect, the invention provides a use of a composition of the invention in the manufacture of a medicament for treating a fungal infection.

The invention also provides a composition for use in treating a fungal infection.

### BRIEF DESCRIPTION OF THE DRAWINGS

FIG. 1 shows the solution stability (as % loss) of AM-2-19 over time. In IV-compatible solvents, such as 5% dextrose in water (“D5W”), AM-2-19 is unstable even over the course of hours.

FIG. 2A shows an image of formulations of AM-2-19-OAc and DSG-PEG-2000 after plasma addition revealing plasma compatibility.

FIG. 2B shows an image of AM-2-19-OAc formulations after plasma addition revealing plasma incompatibility.

FIG. 3 shows the structures of AM-2-19-OAc and DSG-PEG-2000 (“DSGPEG2K”), including the micellar structure of the block copolymer.

FIG. 4 shows overlaid UV spectra of AM-2-19-OAc-DSG-PEG-2000 at 0 h, 3 h, and 24 h. After 24 h, there was a retention >98% of the initial concentration.

FIG. 5A shows overlaid UV spectra of AM-2-19-OAc in D5W at 0 h, 3 h, and 6 h.

FIG. 5B shows overlaid UV spectra of AM-2-19-OAc-DSG-PEG-2000 in D5W at 0 h, 3 h, and 6 h.

FIG. 6A shows a UV spectrum of a 320 μM solution of AM-2-19-OAc in D5W.

FIG. 6B shows overlaid UV spectra of AM-2-19-OAc in D5W at different concentrations.

FIG. 6C shows a UV spectrum of a 320 μM solution of AM-2-19-OAc-DSG-PEG-2000 in D5W.

FIG. 6D shows overlaid UV spectra of AM-2-19-OAc-DSG-PEG-2000 in D5W at different concentrations.

FIG. 7 shows portions of ¹H NMR spectra at various temperatures of deuterated D5W solutions of DSG-PEG-2000 and AM-2-19-OAc-DSG-PEG-2000; also shown is a portion of a ¹H NMR spectrum of a solution of AM-2-19-OAc in deuterated D5W at 25° C.

FIG. 8 contains graphs showing the plasma concentration of the AM-2-19-OAc-DSG-PEG 2000 over time in mouse and rat plasma. As shown, the AM-2-19-OAc-DSG-PEG 2000 formulation exhibits an extended half-life in mice and rats relative to the half-life of AMm-2-19-OAc when not in the micellar formulation.

FIG. 9 contains graphs showing the concentration of the AM-2-19-OAc-DSG-PEG 2000 formulation over time in various tissues.

FIG. 10A shows overlaid UV spectra of 320 μM stock solutions of AM-2-19-OAc and AM-2-19-OAc-DSG-PEG-2000 in D5W.

FIG. 10B shows overlaid UV spectra of RPMI 1640 diluted solution solutions of AM-2-19-OAc and AM-2-19-OAc-DSG-PEG-2000 at 16 μM.

FIG. 10C shows overlaid UV spectra of RPMI 1640 diluted solution solutions of AM-2-19-OAc and AM-2-19-OAc-DSG-PEG-2000 at 4 μM.

FIG. 10D shows overlaid UV spectra of RPMI 1640 diluted solution solutions of AM-2-19-OAc and AM-2-19-OAc-DSG-PEG-2000 at 2 μM.

FIG. 11A shows a UV spectrum of a solution of AM-2-19-OAc in the absence of human plasma and human albumin.

FIG. 11B shows overlaid UV spectra of solutions of AM-2-19-OAc in the presence of human plasma and human albumin respectively.

FIG. 11C shows a UV spectrum of a solution of AM-2-19-OAc-DSG-PEG-2000 in the absence of human plasma and human albumin.

FIG. 11D shows overlaid UV spectra of solutions of AM-2-19-OAc-DSG-PEG-2000 in the presence of human plasma and human albumin respectively.

FIG. 12A shows overlaid UV spectra of solutions of AM-2-19-OAc titrated with Albumin, each solution having a different molar ratio of AM-2-19-OAc to Albumin.

FIG. 12B shows a blown-up portion of the overlaid UV spectra shown in FIG. 12A.

FIG. 12C shows overlaid UV spectra of solutions of AM-2-19-OAc-DSG-PEG-2000 titrated with Albumin, each solution having a different molar ratio of AM-2-19-OAc-DSG-PEG-2000 to Albumin.

FIG. 12D shows a blown-up portion of the overlaid UV spectra shown in FIG. 12C.

FIG. 13 shows the concentration of various toxicity biomarkers in response to the AM-2-19-OAc-DSG-PEG 2000 formulation (API-F100 (1:3)), various control formulations, and other antifungal compositions (e.g., AmBisome-D5W).

FIG. 14 contains graphs showing that AM-2-19-OAc-DSG-PEG 2000 retains the lack of toxicity in vitro observed for AM-2-19-OAc.

FIG. 15 shows a collection of histopathology charts demonstrating that the AM-2-19-OAc-DSG-PEG-2000 formulation does not cause kidney damage in mice, rats, or dogs.

FIG. 16 shows the concentration of various toxicity biomarkers at several time points after a single dose of AM-2-19-OAc-DSG-PEG-2000.

FIG. 17 shows the timeline, clinical pathology of kidney toxicity, and toxicokinetic analysis resulting from a 2-week dosing protocol of AM-2-19-OAc-DSG-PEG-2000 in dogs on Dosing Regimen A (dose every other day).

FIG. 18 shows the timeline, clinical pathology of kidney toxicity, and toxicokinetic analysis resulting from a 2-week dosing protocol of AM-2-19-OAc-DSG-PEG-2000 in dogs on Dosing Regimen B (dose every fourth day).

FIG. 19 shows the timeline, clinical pathology of kidney toxicity, and toxicokinetic analysis resulting from a 2-week dosing protocol of AM-2-19-OAc-DSG-PEG-2000 in dogs on Dosing Regimen C (dose every week).

FIG. 20 shows the results of efficacy assessments showing that AM-2-19-OAc-DSG-PEG-2000 exhibits a lower minimum inhibitory concentration (MIC) than the Amphotericin B liposome formulation AmBisome against numerous strains of yeasts and moulds, including resistance refractory C. albicans ATCC 90028.

FIG. 21 contains a series of graphs that show that AM-2-19-OAc-DSG-PEG-2000 decreases the fungal burden of numerous fungal pathogens in lung and kidney tissue as compared to control and to AmBisome.

FIG. 22 contains overlaid UV spectra of the DSG-PEG-2000 micellar formulations of several AmB derivatives at various time points showing stability of the formulations.

### DETAILED DESCRIPTION OF THE INVENTION

The present invention is based on the discovery of a micellar formulation of Amphotericin B and derivatives thereof that provides improved solution stability and plasma concentration of the antifungal payload. The inventions surprisingly discovered that the formulation unexpectedly increases the potency of the Amphotericin derivative, and also extends its half-life in vivo.

Amphotericin B (AmB) is a polyene macrolide with a mycosamine appendage, the complete compound has the structure below.

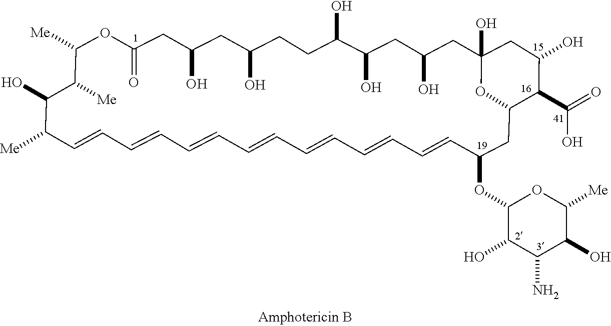 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

AmB is generally obtained from a strain of Streptomyces nodosus. It is currently approved for clinical use in the United States for the treatment of progressive, potentially life-threatening fungal infections, including such infections as systemic or deep tissue candidiasis, aspergillosis, cryptococcosis, blastomycosis, coccidioidomycosis, histoplasmosis, and mucormycosis, among others. It is generally formulated for intravenous injection. Amphotericin B is commercially available, for example, as Fungizone® (Squibb), Amphocin® (Pfizer), Abelcet® (Enzon), and Ambisome® (Astellas). Due to its undesirable toxic side effects, dosing is generally limited to a maximum of about 1.0 mg/kg/day and total cumulative doses not to exceed about 3 g in humans.

AmB kills both fungal and human cells by forming a cytocidal extramembranous sterol sponge. Anderson, T. M. et al., Nat Chem Biol 2014, 10 (5), 400-6. This large aggregate sits on the surface of lipid bilayers and rapidly extracts membrane sterols, which leads to cell death. Membrane permeabilization is not required. Based on this mechanism, a small molecule-based ligand-selective allosteric effect would enable selective binding of ergosterol over cholesterol and would eliminate the mammalian toxicity of AmB (in the form of C2′epiAmB). See Wilcock, B. C. et al., J Am Chem Soc 2013, 135 (23), 8488-91. The present invention discloses the K_{D}s for the binding of both ergosterol and cholesterol to the AmB sterol sponge, which provides a quantitative and mechanistically-grounded biophysical parameter to guide rational optimization of the therapeutic index of this clinically significant natural product.

Further derivatives of AmB have been identified and been shown to have an improved therapeutic index compared to AmB. Such derivatives of AmB retain potent binding of ergosterol but show no detectable binding of cholesterol, and retain fungicidal potency against many yeasts and molds but shows no detectable mammalian toxicity. This demonstrates that differential binding of ergosterol over cholesterol is possible and provides non-toxic variants of AmB that preserve desirable antifungal properties.

Though such AmB derivatives have therapeutic potential to eradicate life-threatening invasive fungal infections with a significantly improved safety profile, such compounds also suffer from poor plasma compatibility and solution instability, which presents challenges for drug administration, particularly intravenous administration.

As such, this invention is based in part on the discovery of new formulations of AmB derivatives which (1) are efficacious against fungal pathogens, (2) minimize mammalian toxicity, (3) exhibit plasma compatibility, and (4) are stable in solution.

Compositions of the invention are useful for inhibiting the growth of a fungus. In one embodiment, an effective amount of a composition of the invention is contacted with a fungus, thereby inhibiting growth of the fungus. In one embodiment, a composition of the invention is added to or included in tissue culture medium.

Compositions of the invention are useful for the treatment of fungal infections in a subject. In one embodiment, a therapeutically effective amount of a composition of the invention, is administered to a subject in need thereof, thereby treating the fungal infection.

Yeasts are eukaryotic organisms classified in the kingdom Fungi. Fungi include yeasts, molds, and larger organisms including mushrooms. Yeasts and molds are of clinical relevance as infectious agents. Yeasts are typically described as budding forms of fungi. Of particular importance in connection with the invention are species of yeast that can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic yeasts include, without limitation, various species of the genus Candida, as well as of Cryptococcus. Of particular note among pathogenic yeasts of the genus Candida are C. albicans, C. tropicalis, C. stellatoidea, C. glabrata, C. krusei, C. parapsilosis, C. guilliermondii, C. viswanathii, and C. lusitaniae. The genus Cryptococcus specifically includes Cryptococcus neoformans. Yeast can cause infections of mucosal membranes, for example oral, esophageal, and vaginal infections in humans, as well as infections of bone, blood, urogenital tract, and central nervous system. This list is exemplary and is not limiting in any way.

A number of fungi (apart from yeast) can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic fungi (apart from yeast) include, without limitation, species of Aspergillus, Rhizopus, Mucor, Histoplasma, Coccidioides, Blastomyces, Trichophyton, Microsporum, and Epidermophyton. Of particular note among the foregoing are A. fumigatus, A. flavus, A. niger, H. capsulatum, C. immitis, and B. dermatitidis. Fungi can cause systemic and deep tissue infections in lung, bone, blood, urogenital tract, and central nervous system, to name a few. Some fungi are responsible for infections of the skin and nails.

### Compositions of the Invention

In certain aspects, the invention provides a composition, comprising:
(i) a lipid polymer excipient having the structure of formula (X);

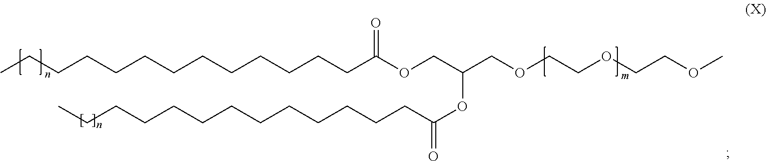

wherein each occurrence of n is independently selected from 0-10; and
m is selected from 10-60; and
(ii) a compound, or a pharmaceutically acceptable salt thereof, selected from the group consisting of:

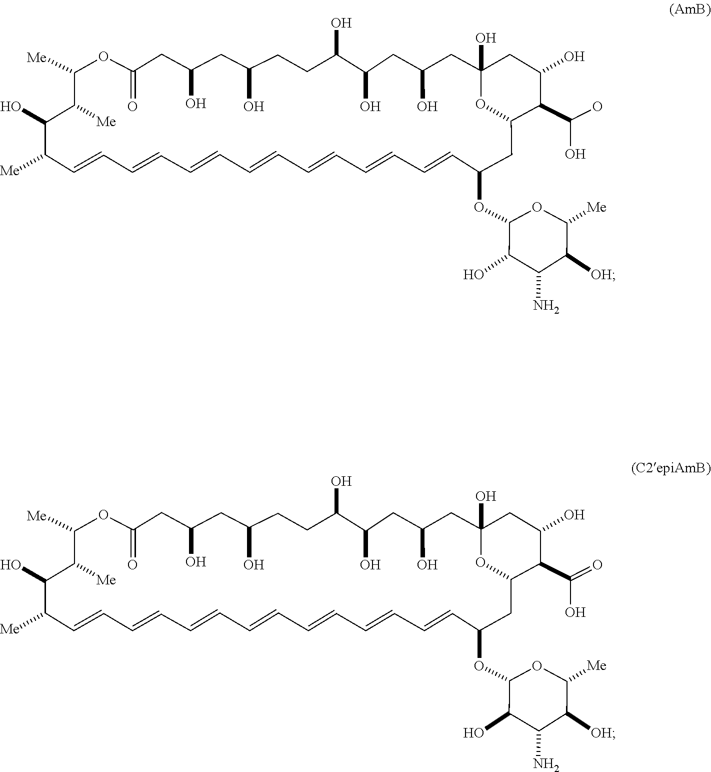

a compound having the structure of formula (I):

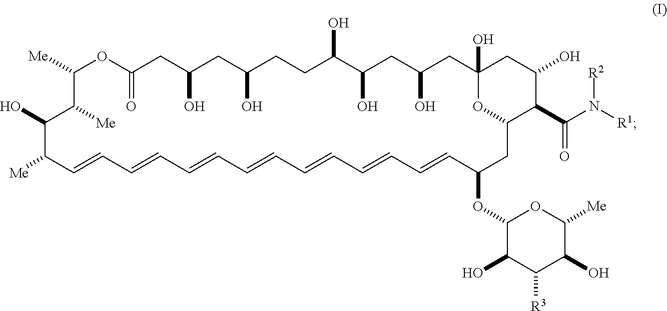

and
a compound having the structure of formula (II):

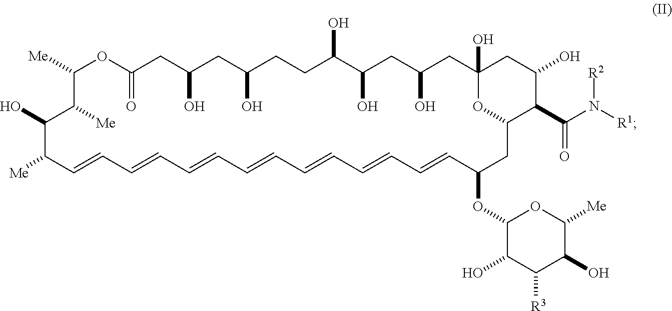

wherein:
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², taken together with the nitrogen to which they are attached, form a substituted or unsubstituted 3- to 10-membered heterocyclyl;
R³ is —NR⁵R⁶, substituted or unsubstituted amino, substituted or unsubstituted urea, substituted or unsubstituted carbamate or substituted or unsubstituted guanidinyl;
R⁴ is hydrogen or substituted or unsubstituted C₁₋₆ alkyl;
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, taken together with the nitrogen to which they are attached, form a substituted or unsubstituted 3- to 10-membered heterocyclyl; and
R^{f} is selected from the group consisting of 2-alken-1-yl, tert-butyl, benzyl and fluorenylmethyl.

In certain embodiments, the compound is AmB.

In certain embodiments, the compound is C2′epiAmB.

In certain embodiments, the compound is a compound having the structure of formula (I).

In certain embodiments, the compound is a compound having the structure of formula (II).

In certain embodiments, the compound is a compound having the structure of formula (I) or formula (II); and R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

In certain embodiments, the compound is a compound having the structure of formula (I) or formula (II); and R¹ and R² independently are hydrogen, unsubstituted C₁₋₆ alkyl, hydroxyl C₁₋₆ alkyl, alkoxy C₁₋₆ alkyl, halo C₁₋₆ alkyl, amino C₁₋₆ alkyl, heterocyclyl C₁₋₆ alkyl, unsubstituted C₂₋₆ alkynyl, unsubstituted C₃₋₁₀ carbocyclyl, amino C₃₋₁₀ carbocyclyl, unsubstituted 3- to 10-membered heterocyclyl, or hydroxyl 3- to 10-membered heterocyclyl.

In certain embodiments, the compound is a compound having the structure of formula (I) or formula (II); and at least one of R¹ and R² is hydrogen.

In certain embodiments, the compound is a compound having the structure of formula (I) or formula (II); and R¹ and R² are not both hydrogen.

In certain embodiments, the compound is a compound having the structure of formula (I) or formula (II); and R¹ and R², taken together with the nitrogen to which they are attached, form a substituted or unsubstituted 3- to 10-membered heterocyclyl.

In certain embodiments, the compound is a compound having the structure of formula (I) or formula (II);
R³ is —NR⁵R⁶;
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, taken together with the nitrogen to which they are attached, form a substituted or unsubstituted 3- to 10-membered heterocyclyl; and
R^{f} is selected from the group consisting of 2-alken-1-yl, tert-butyl, benzyl and fluorenylmethyl.

In certain such embodiments, R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

In further such embodiments, R⁵ and R⁶ independently are hydrogen or C(O)OR^{f}, optionally wherein R^{f} is fluorenylmethyl. In certain such embodiments, at least one of R⁵ and R⁶ is hydrogen; preferably, R⁵ and R⁶ are both hydrogen.

In certain embodiments, the compound is a compound having the structure of formula (I) or formula (II); and R⁴ is hydrogen, substituted or unsubstituted C₁₋₆ alkyl, or substituted or unsubstituted C₂₋₆ alkenyl. In certain such embodiments, R⁴ is hydrogen, halo C₁₋₆ alkyl, or unsubstituted C₂₋₆ alkenyl. In certain preferred embodiments, R⁴ is hydrogen.

In certain embodiments, the compound is selected from the group consisting of:

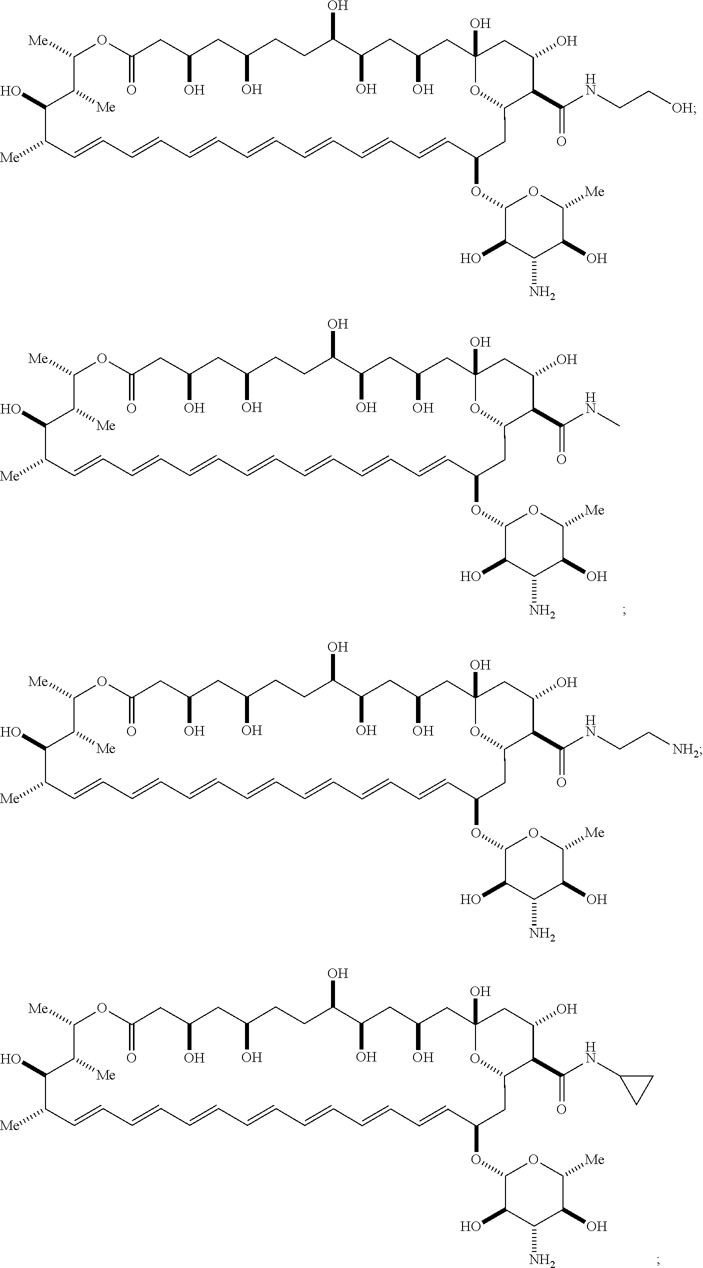

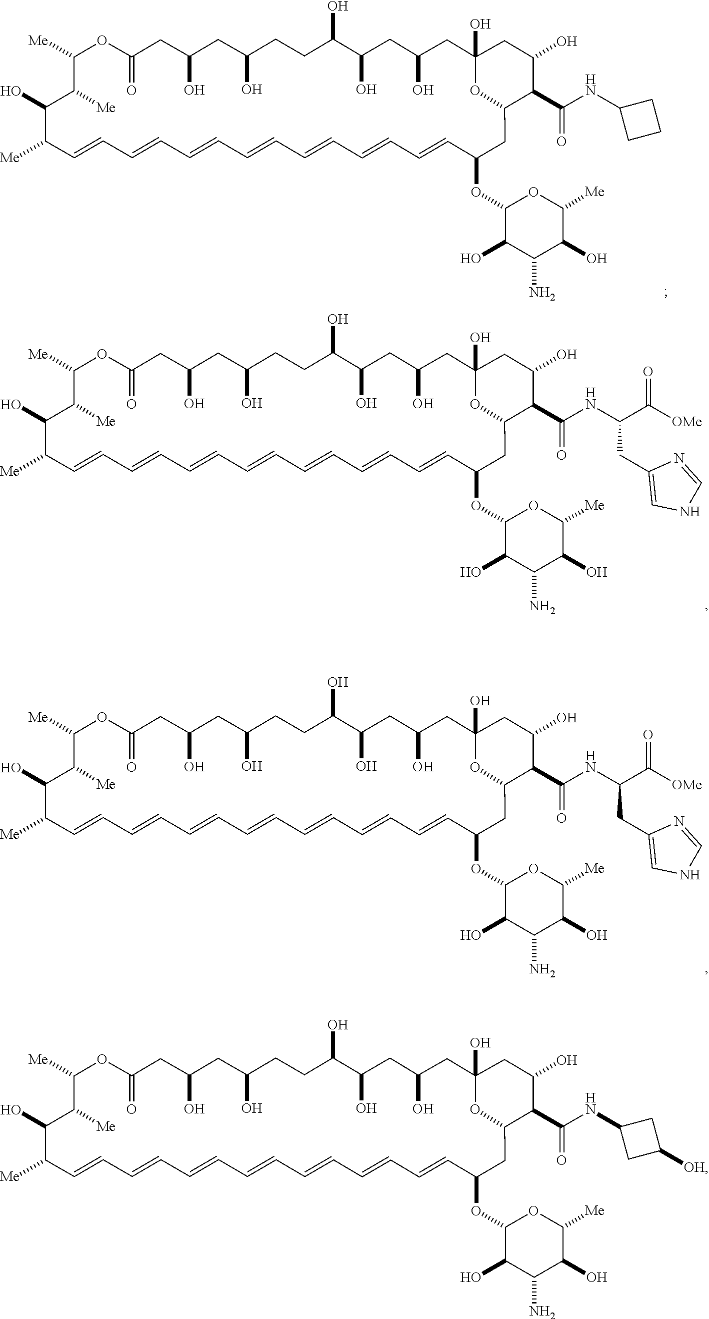

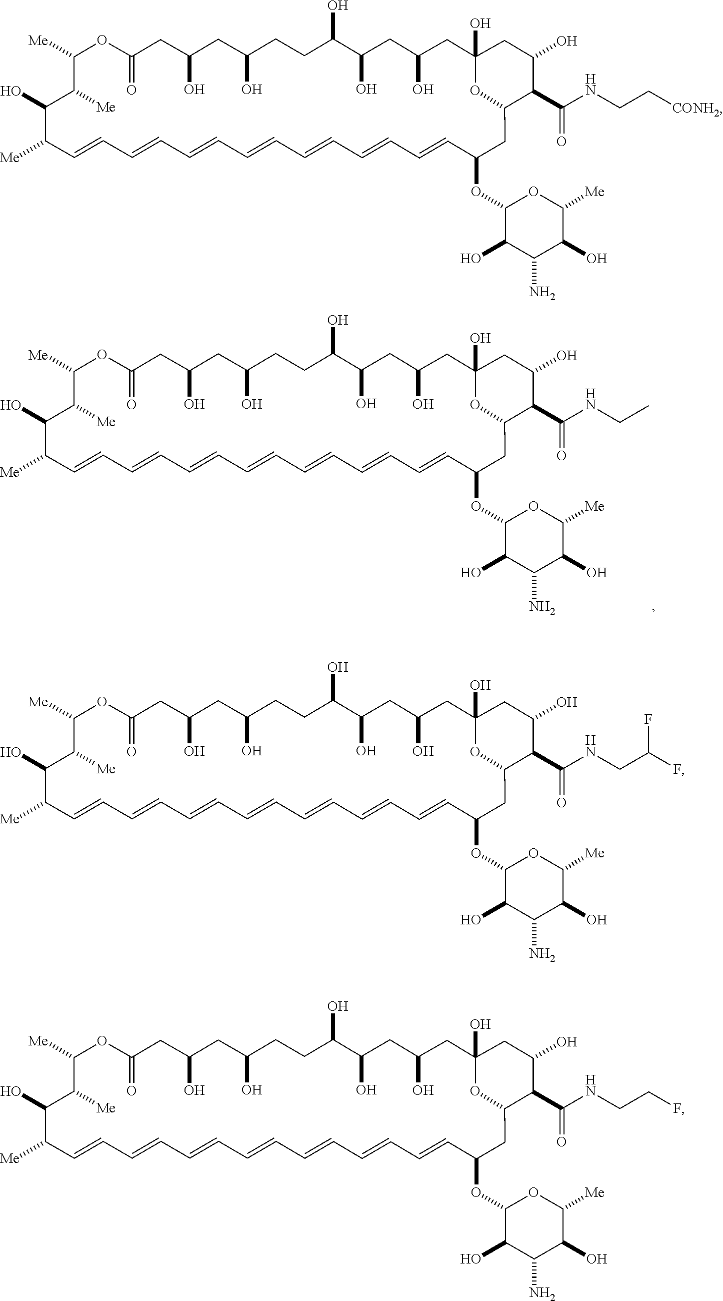

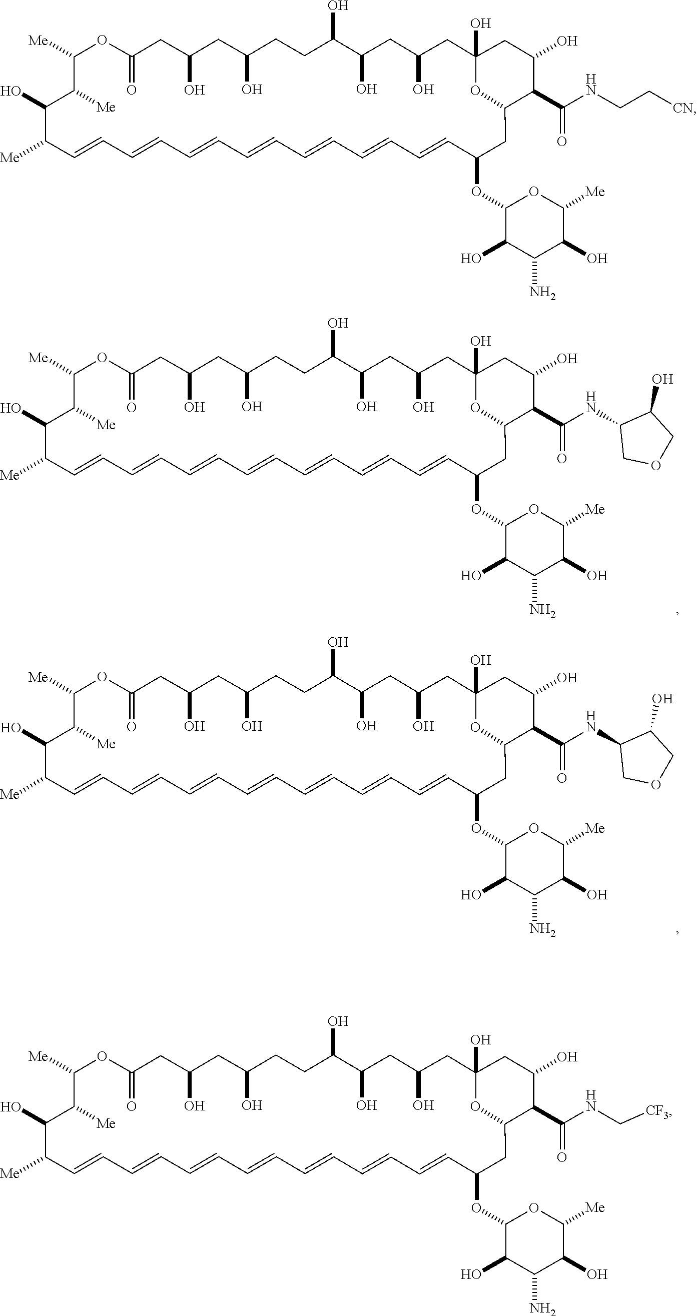

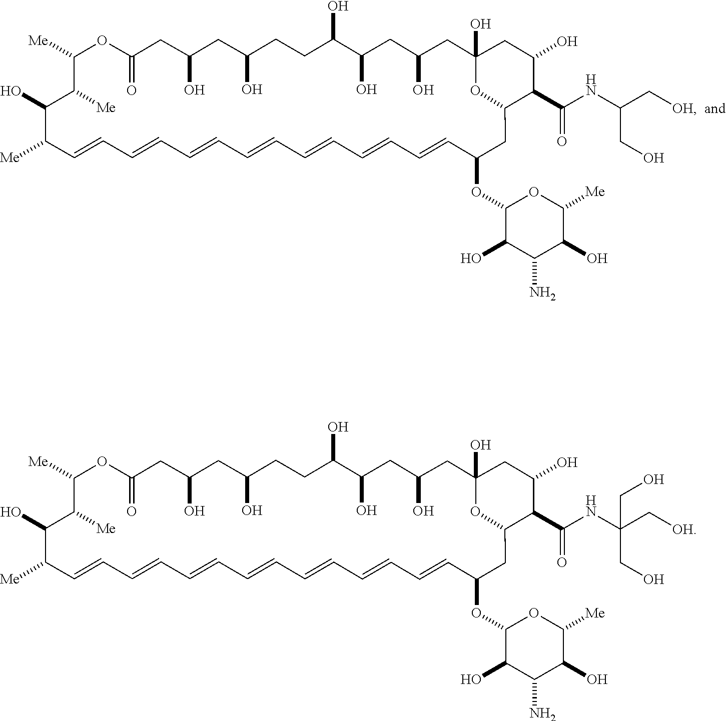

Alternatively, the compound is selected from the group consisting of:

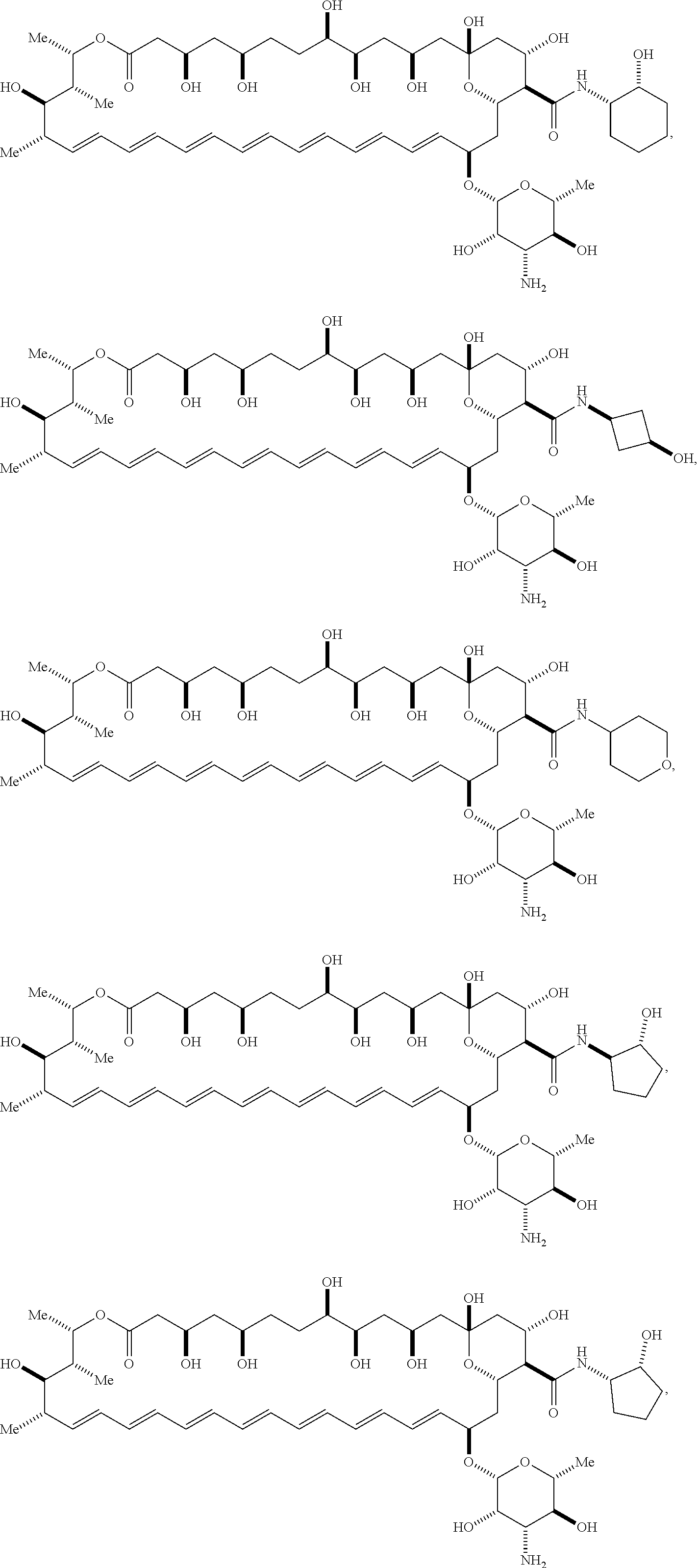

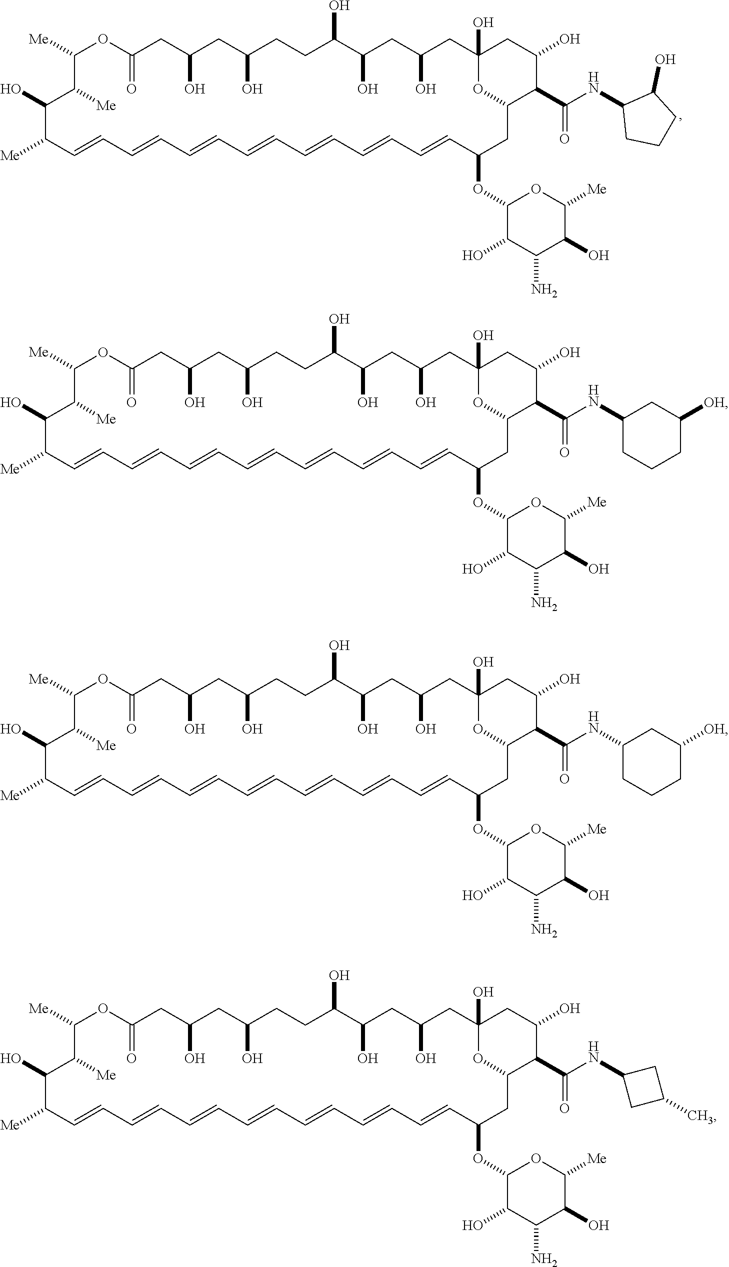

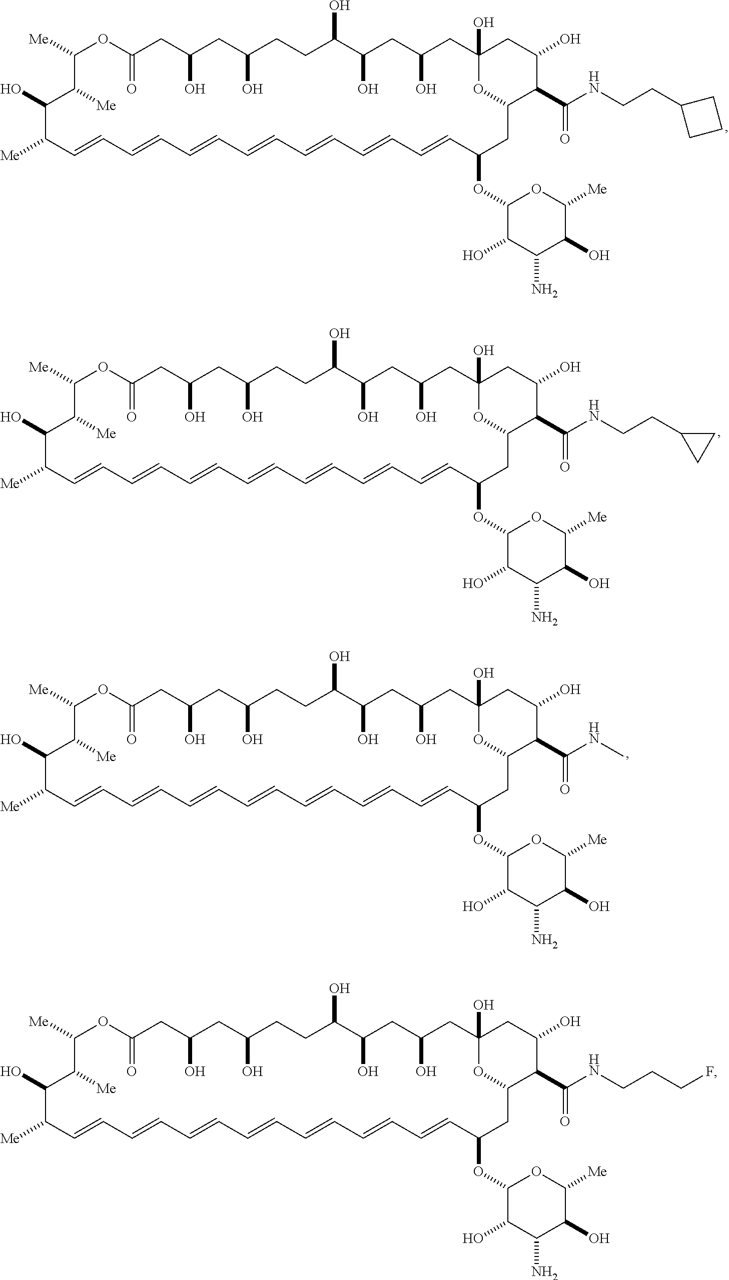

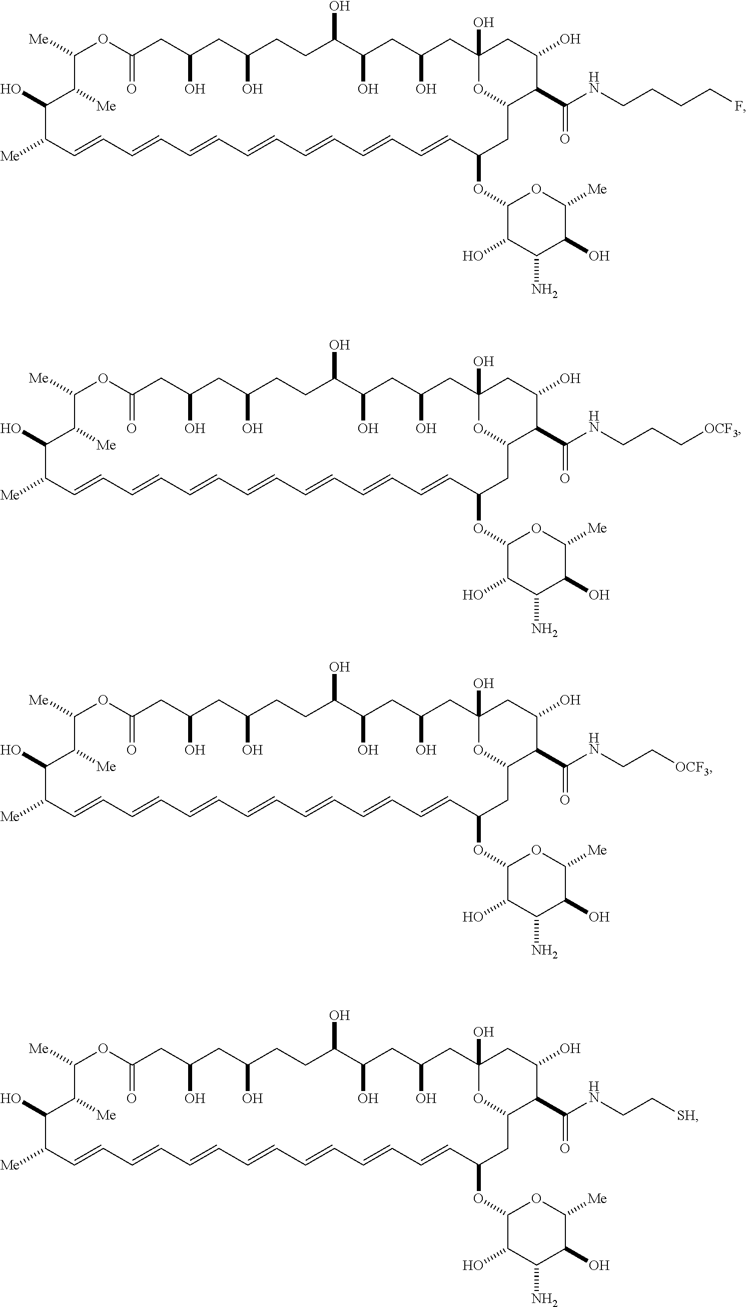

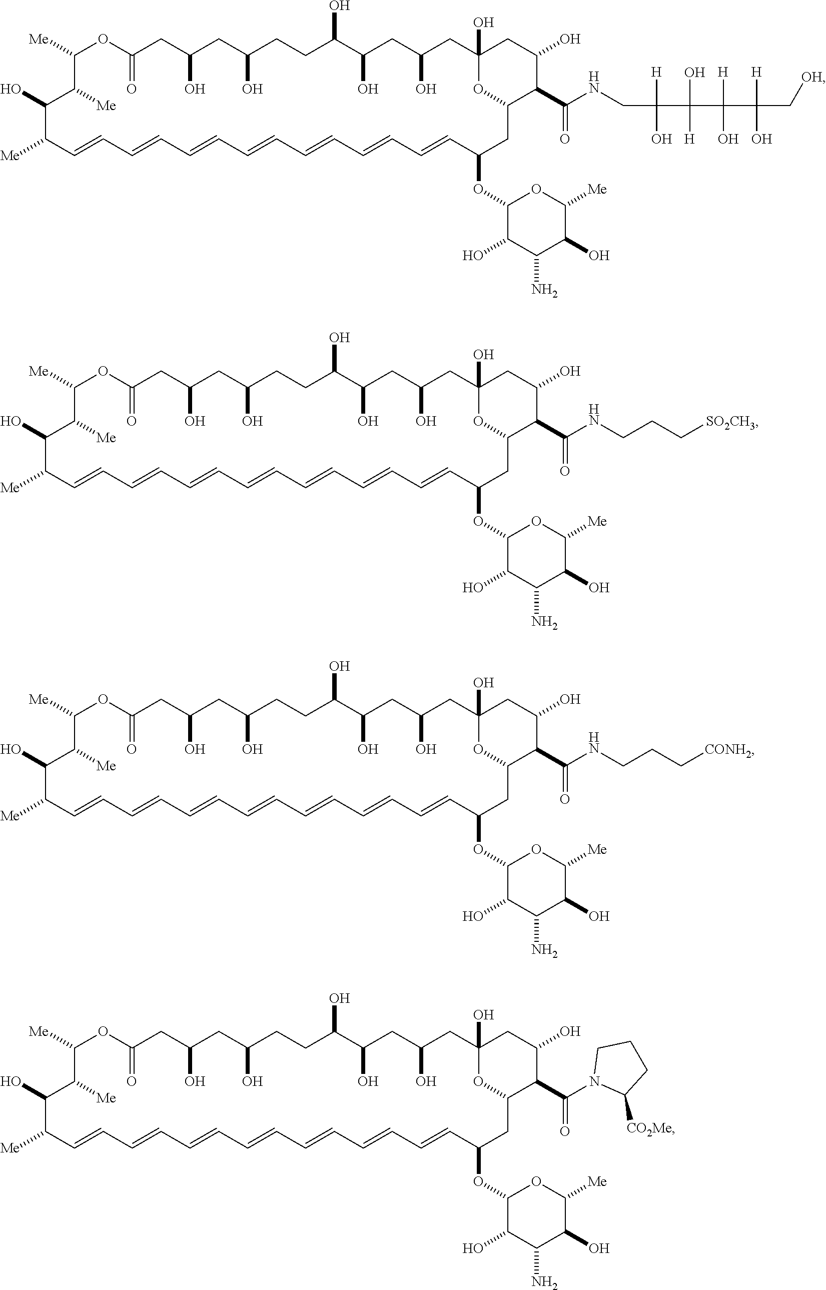

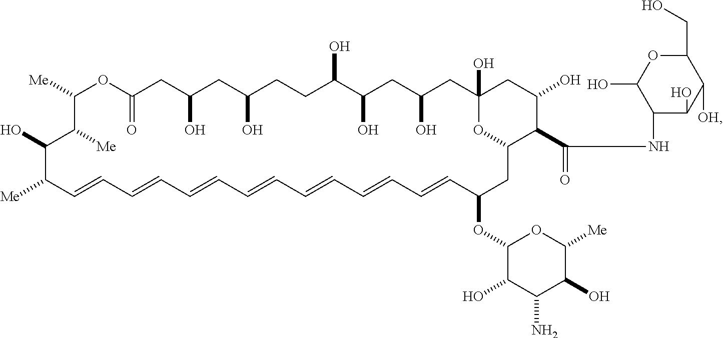

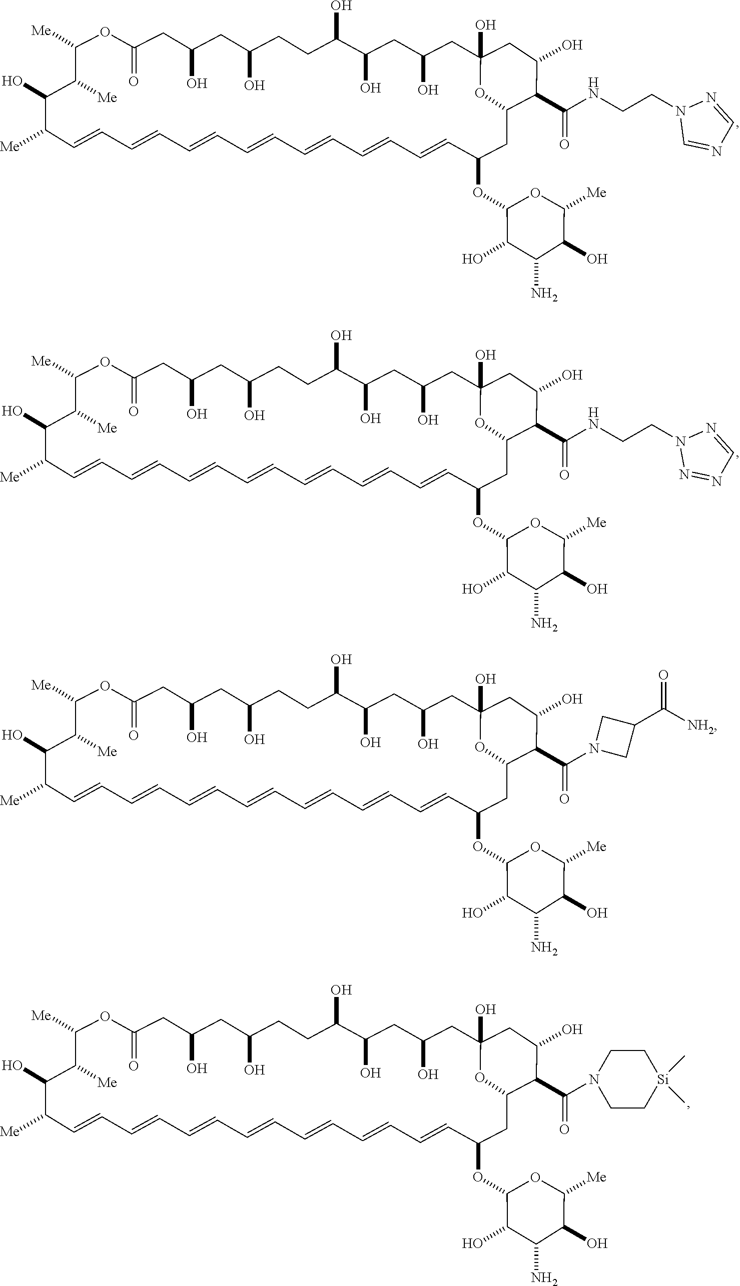

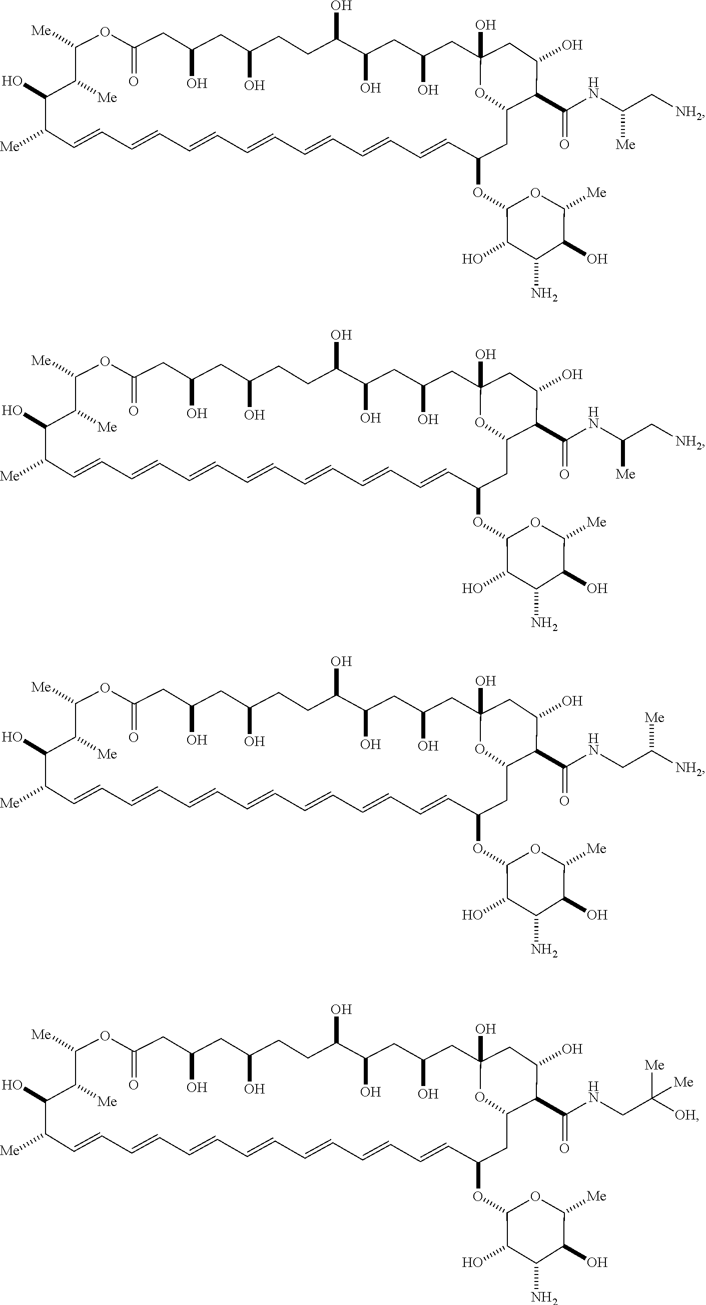

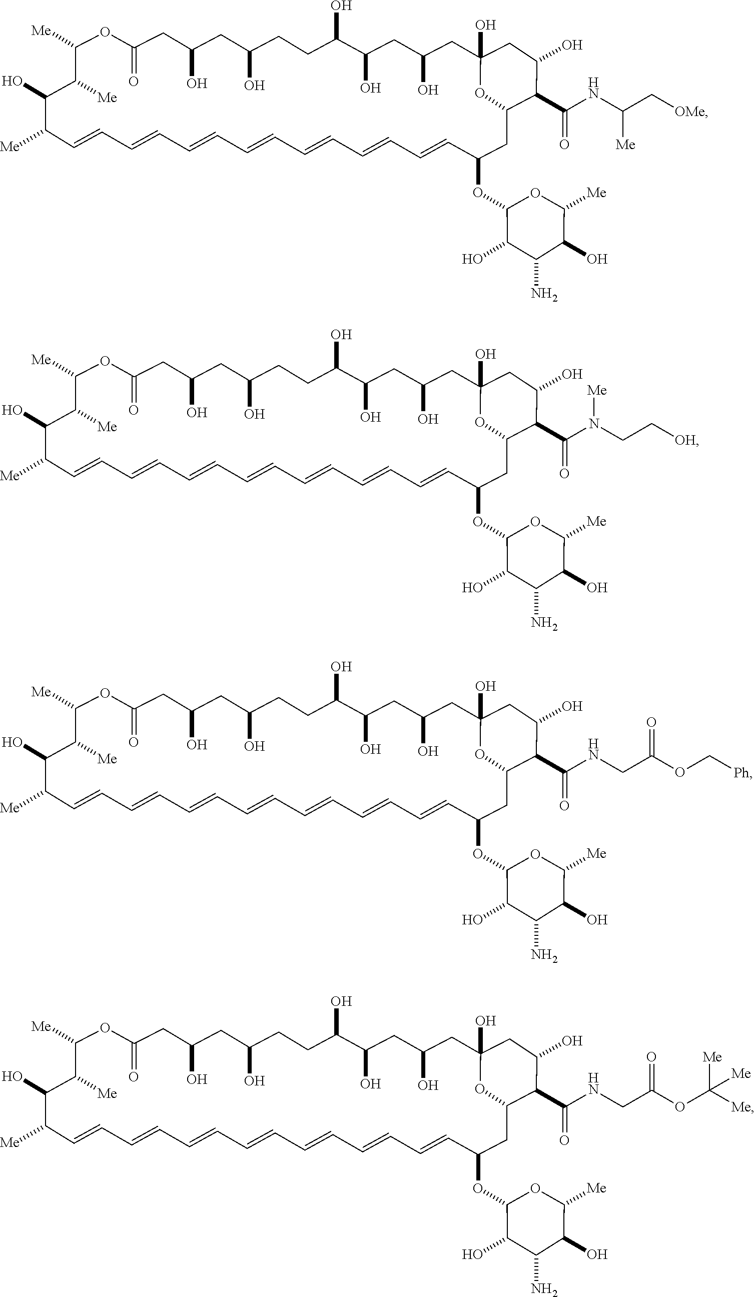

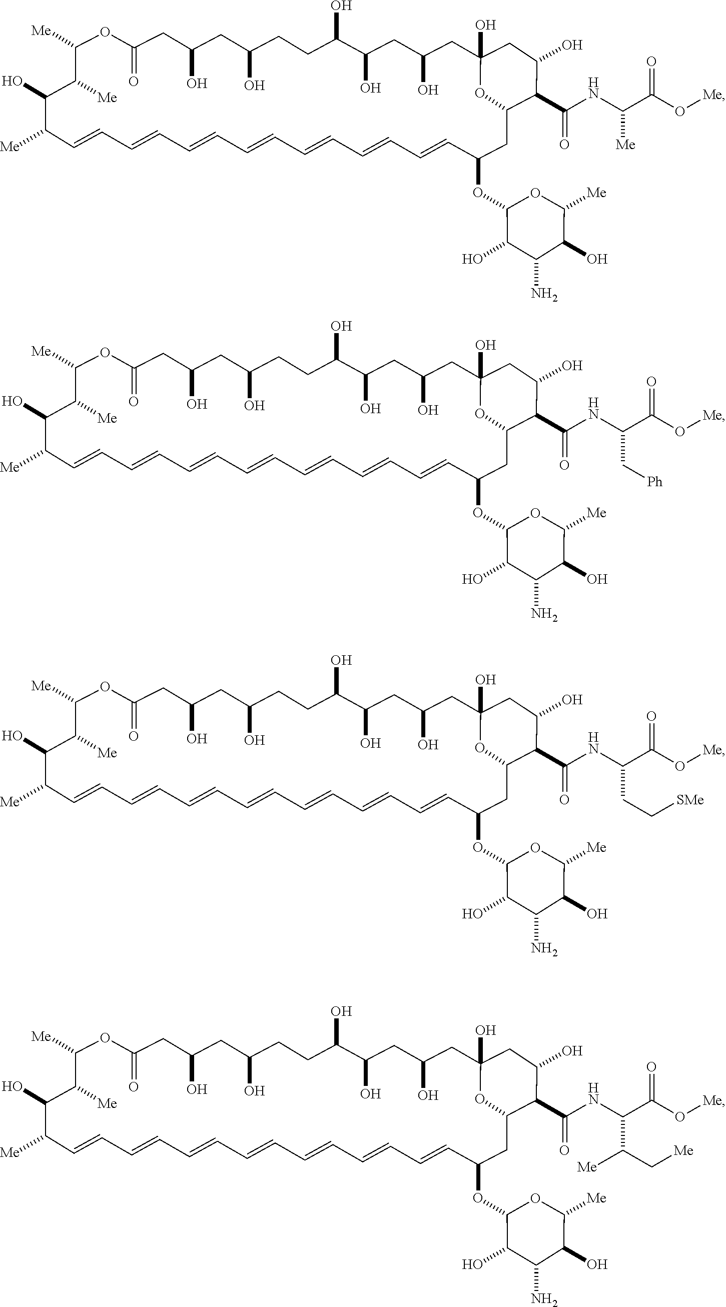

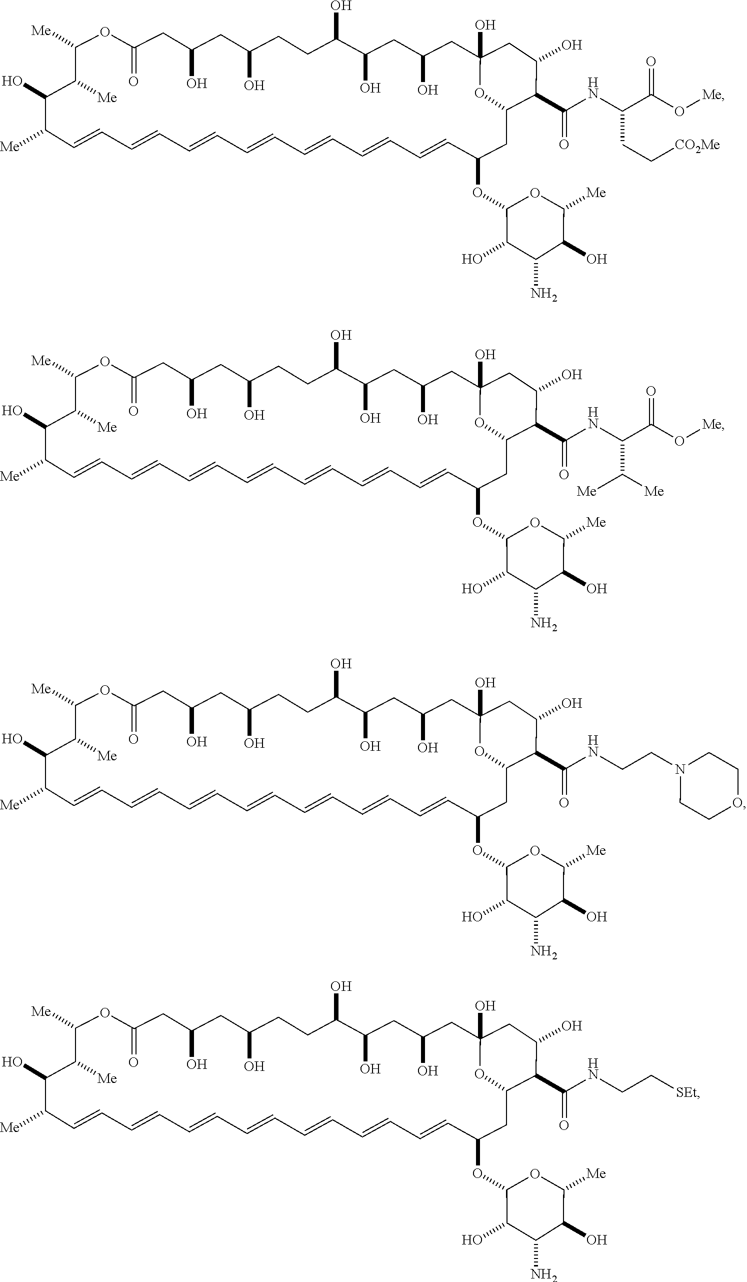

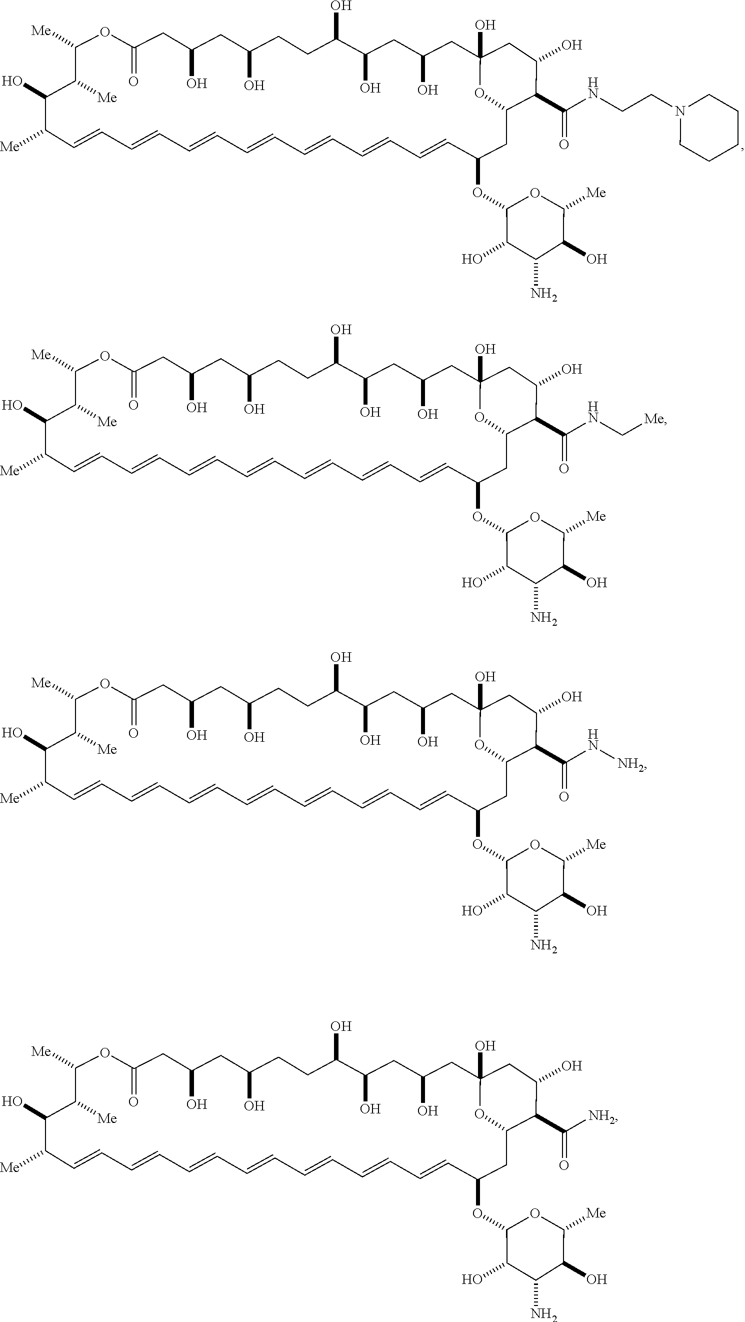

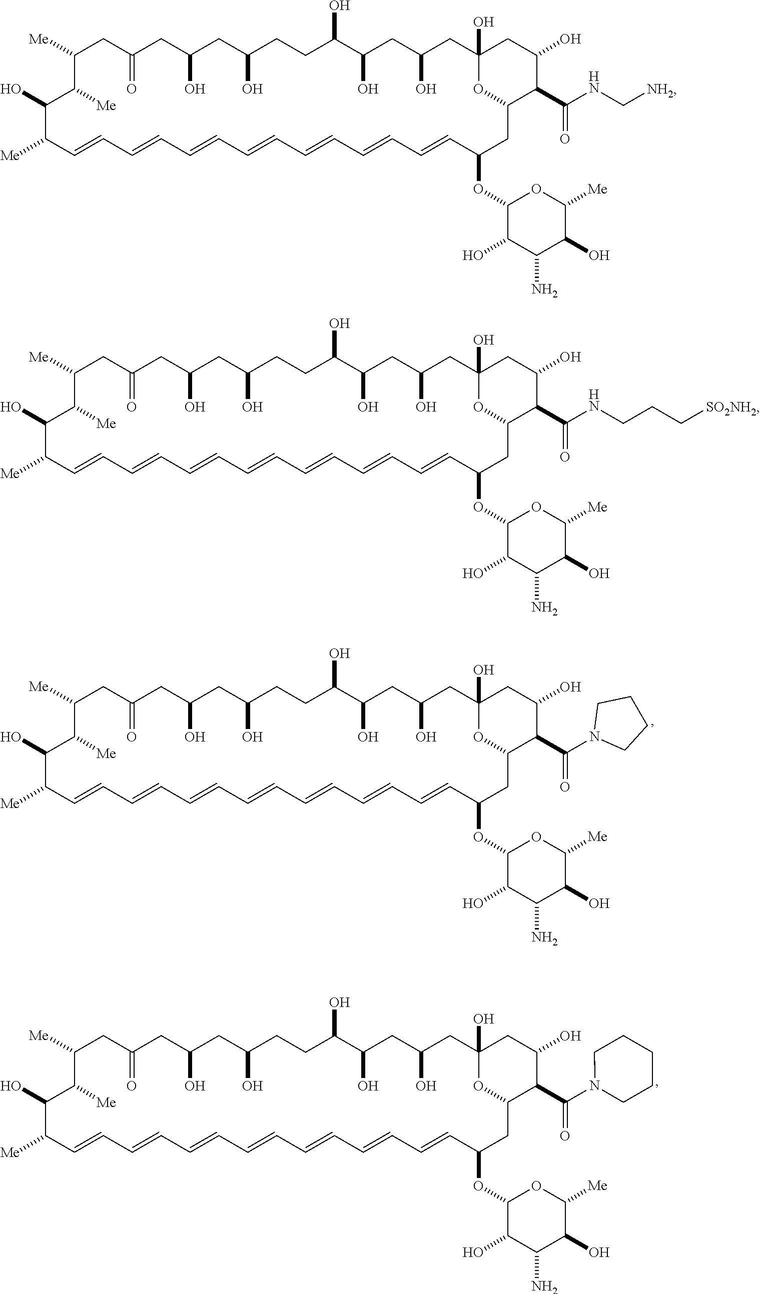

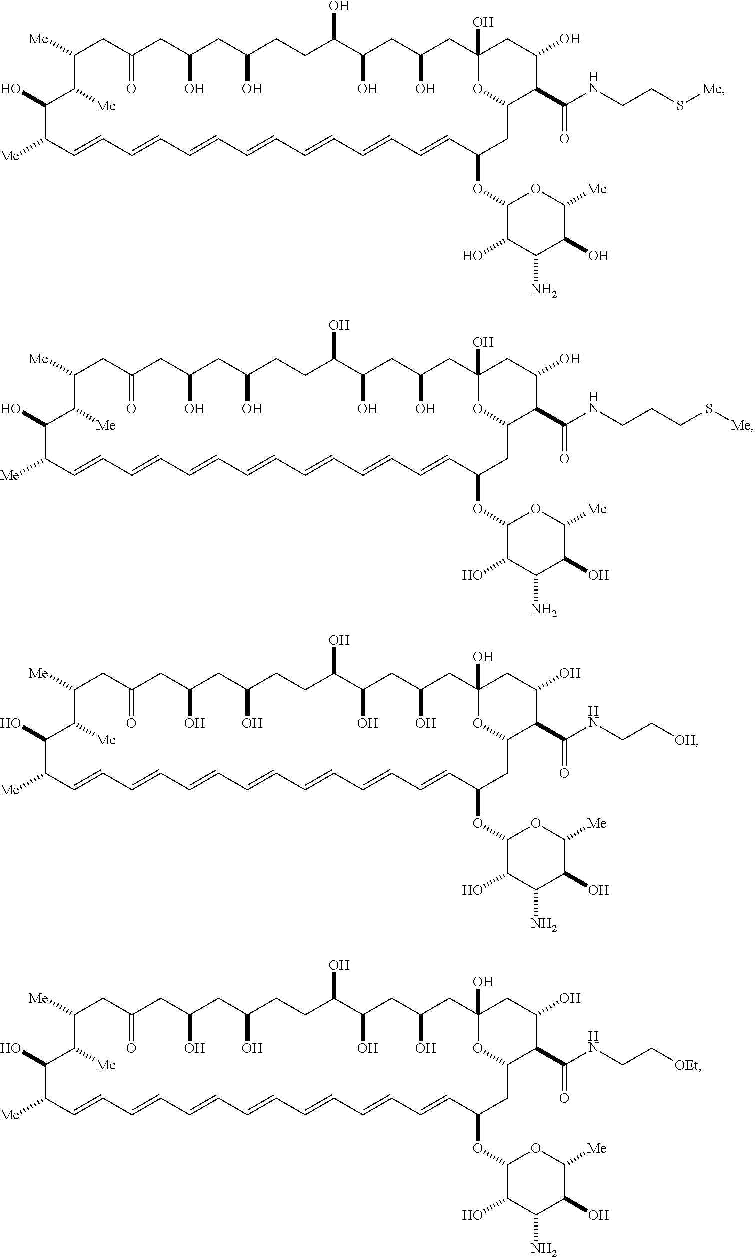

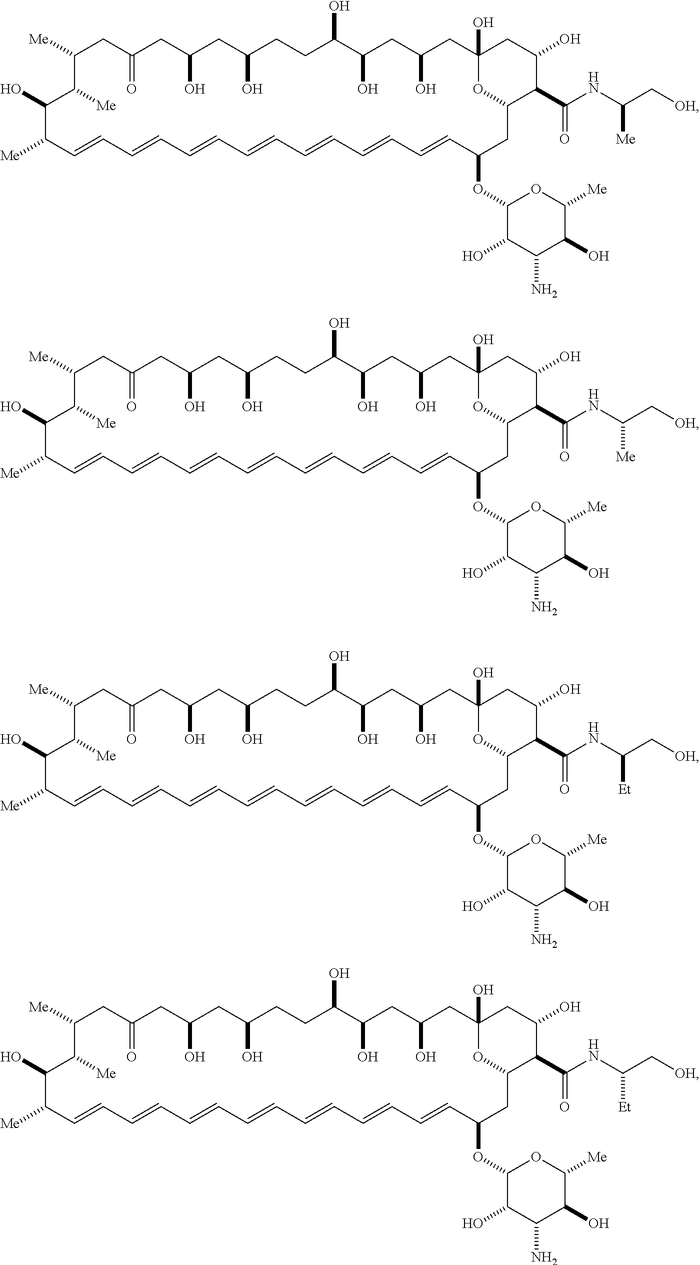

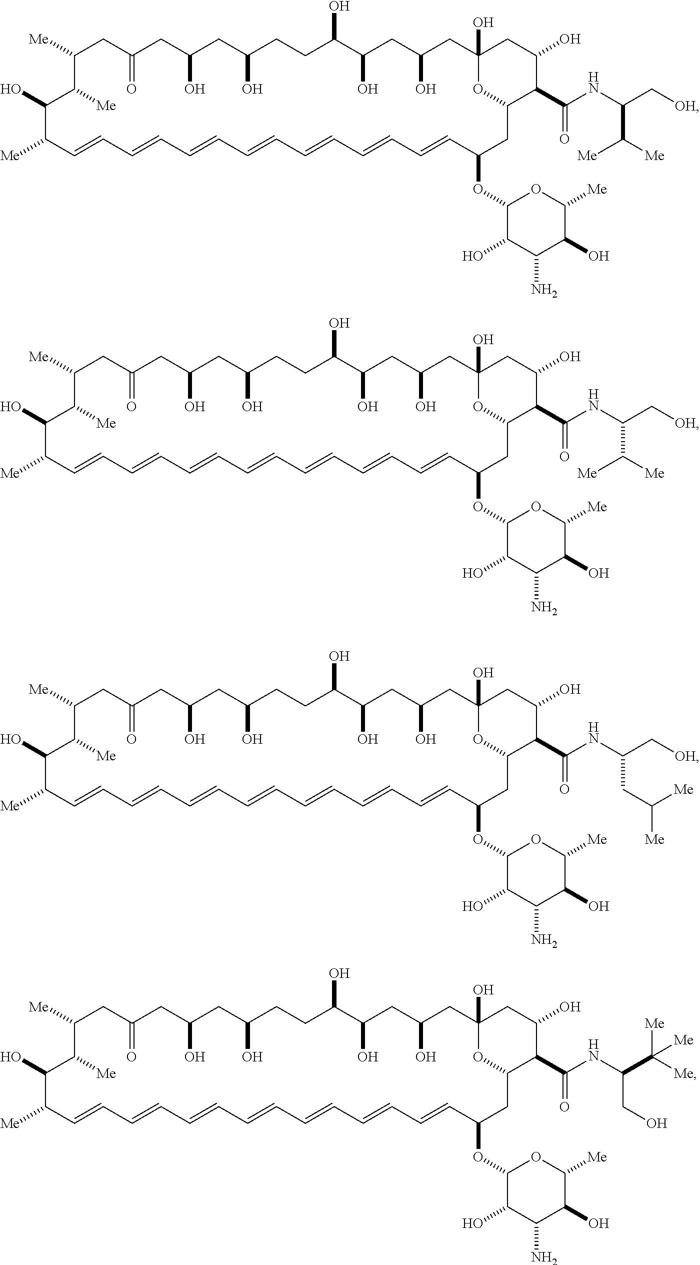

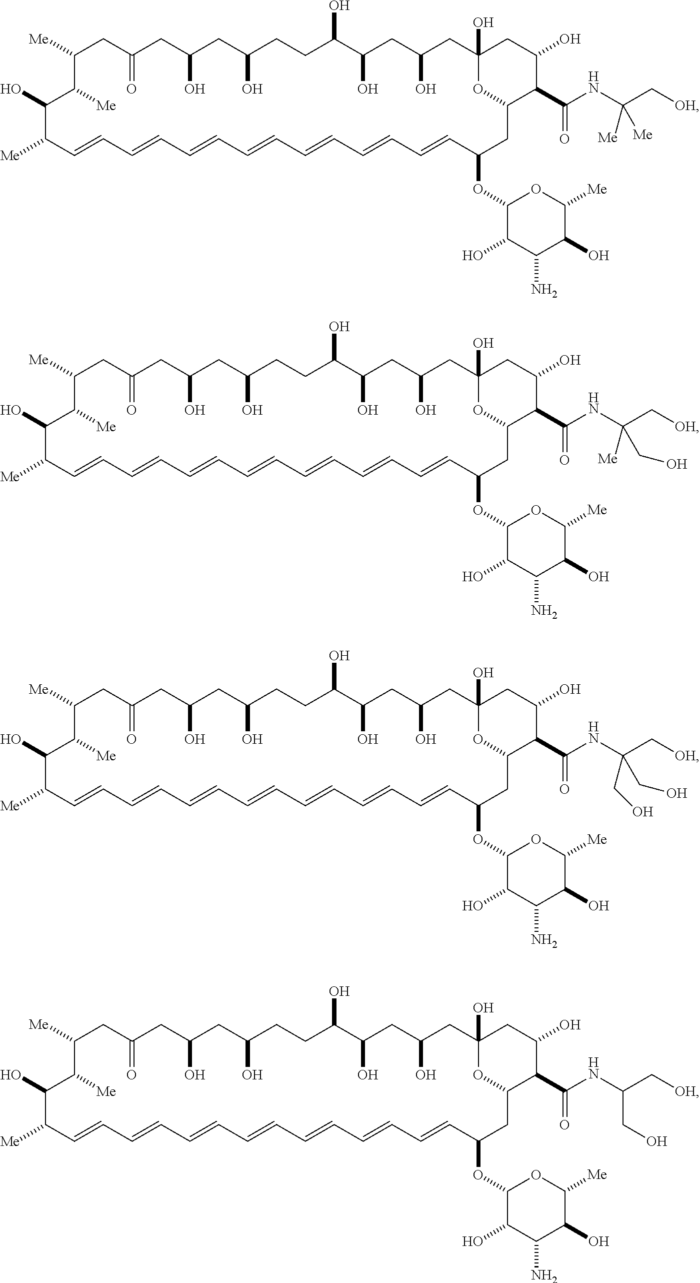

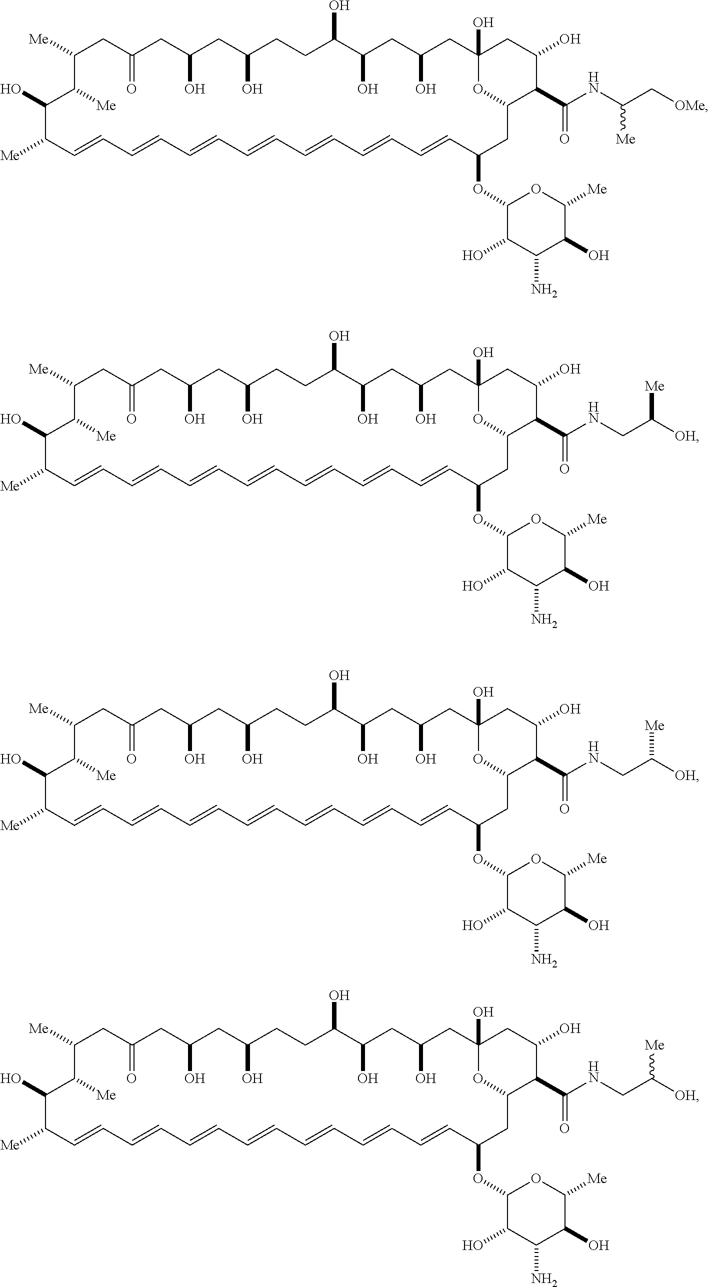

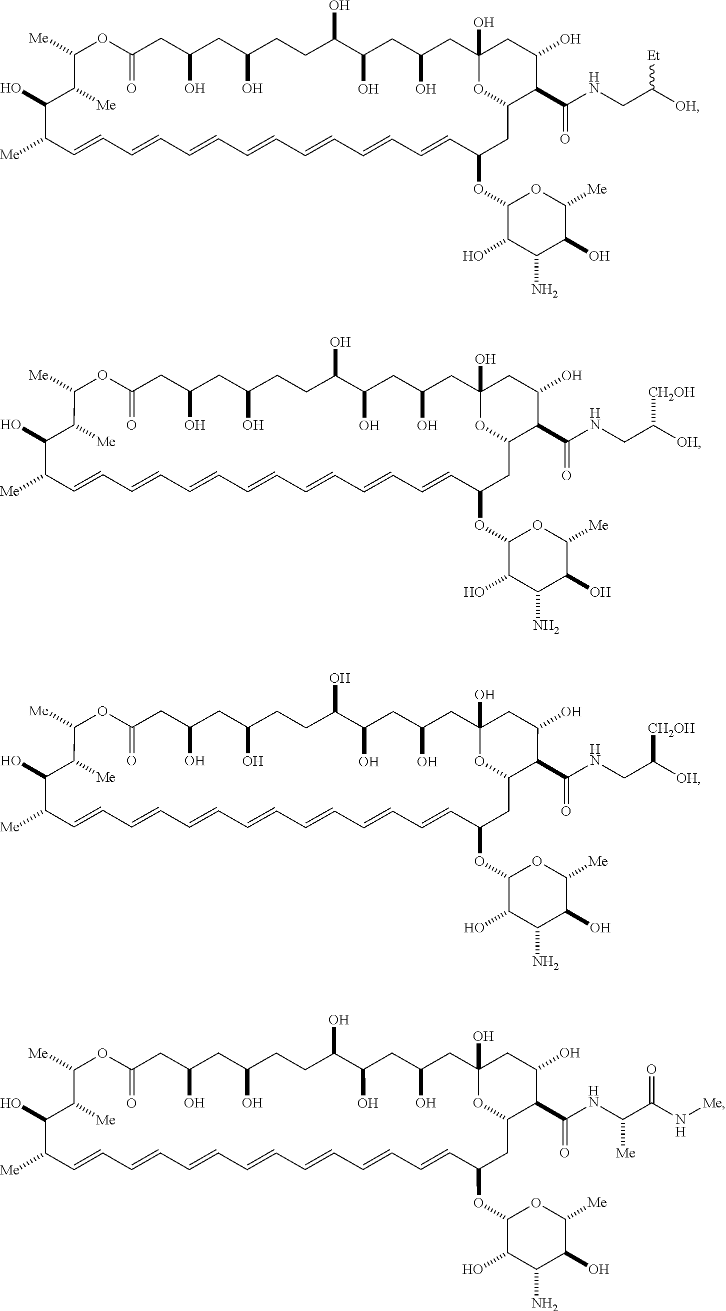

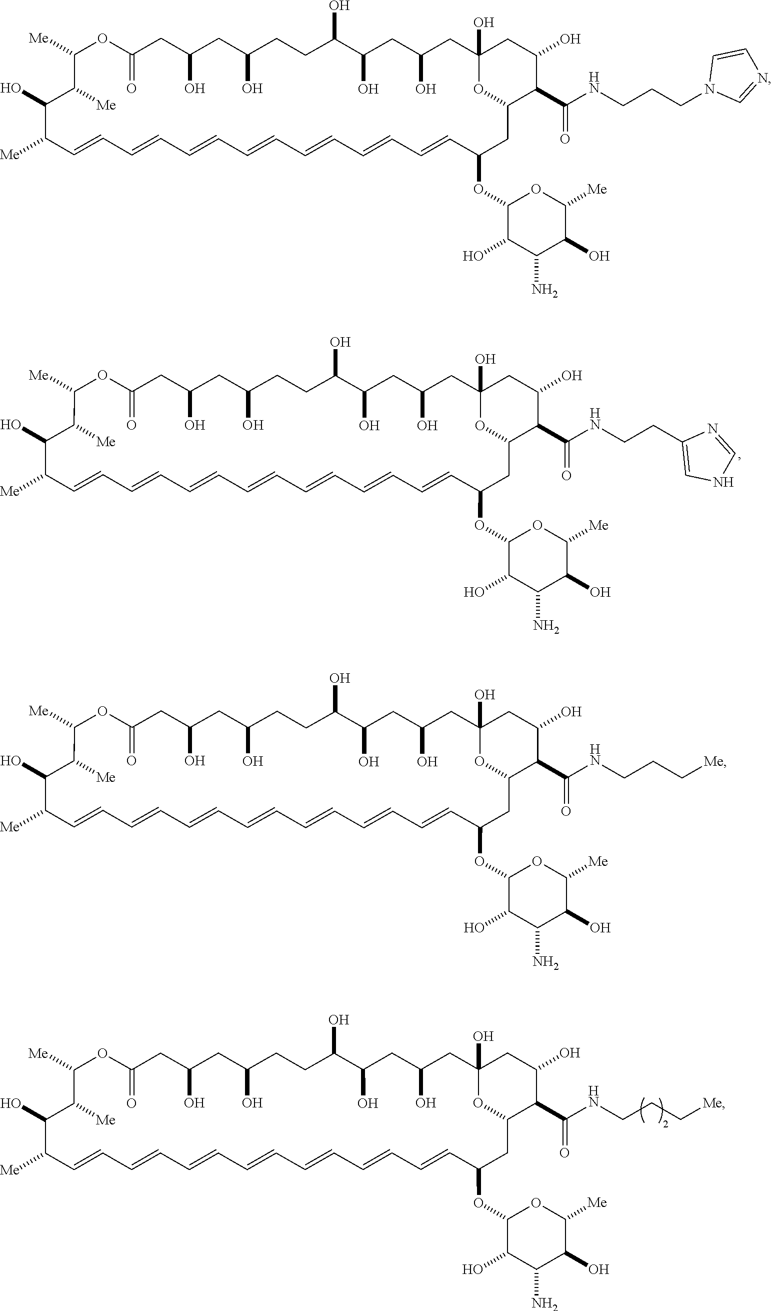

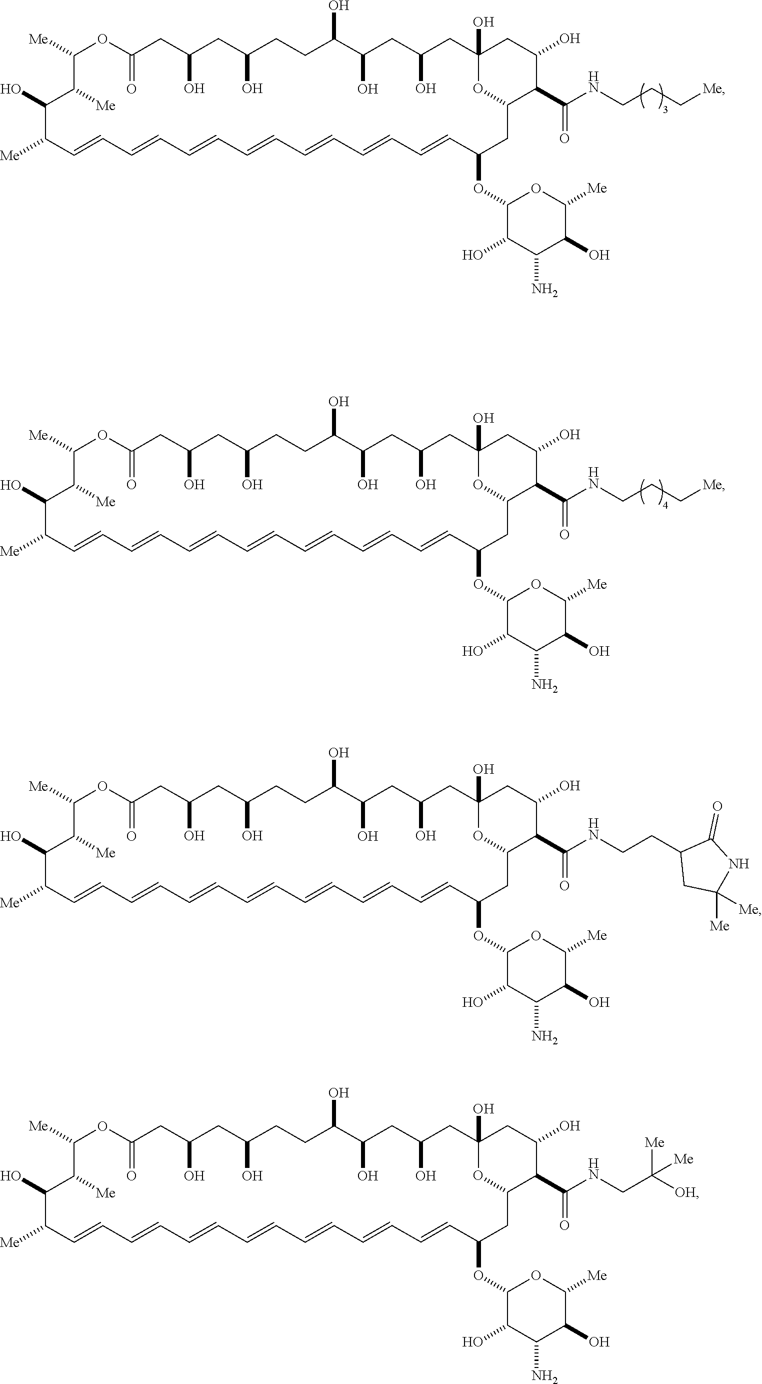

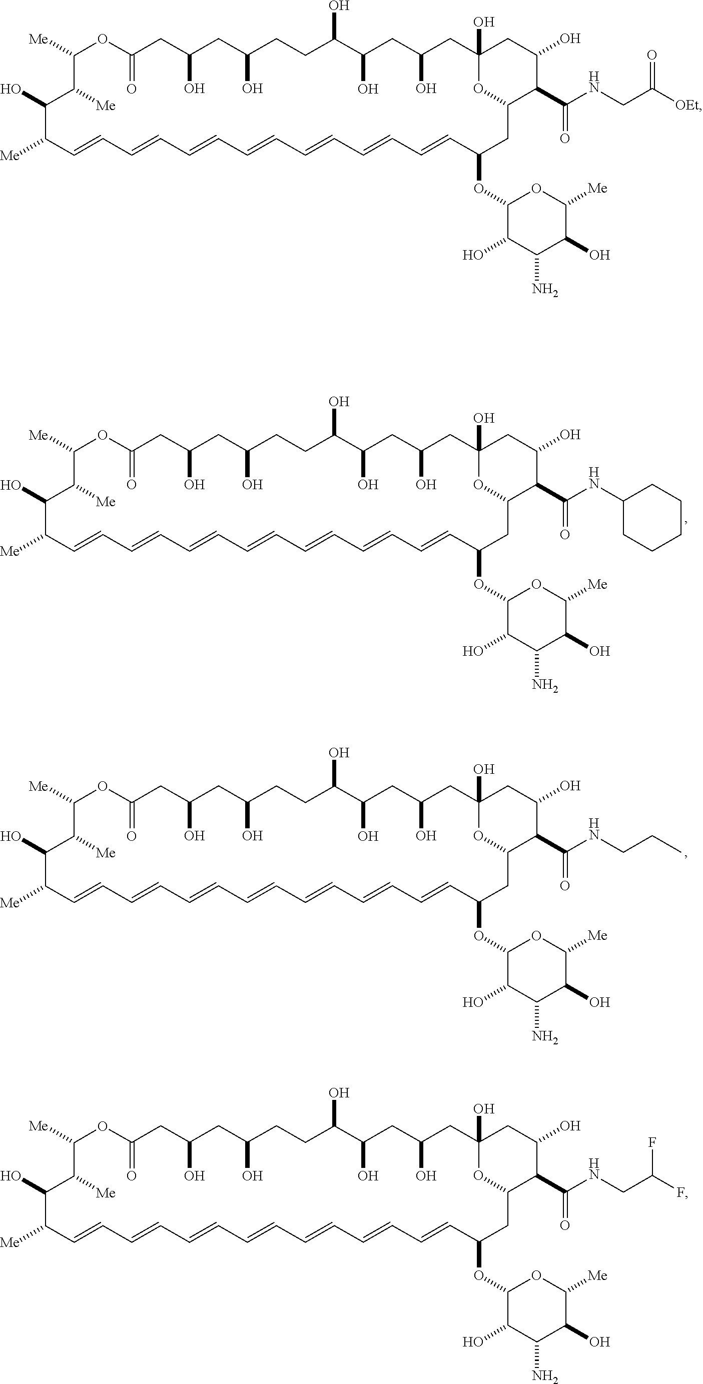

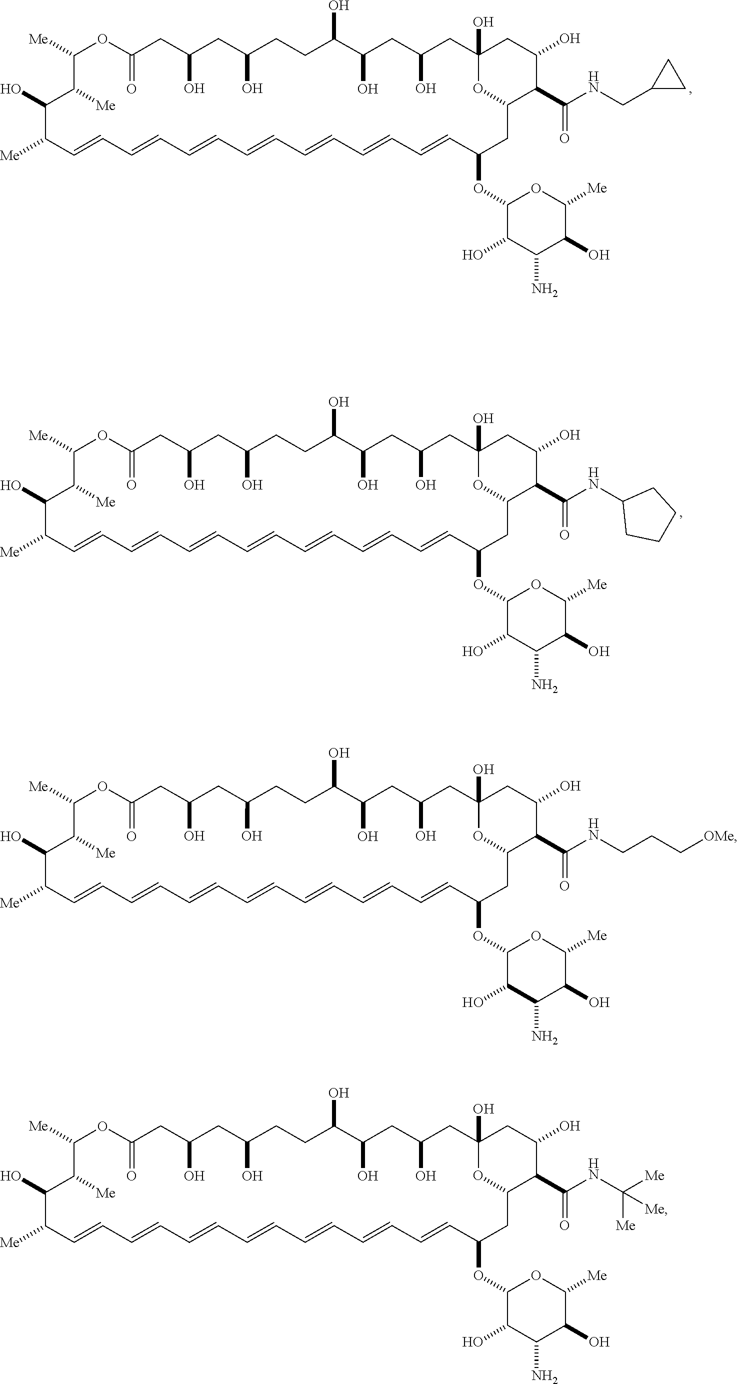

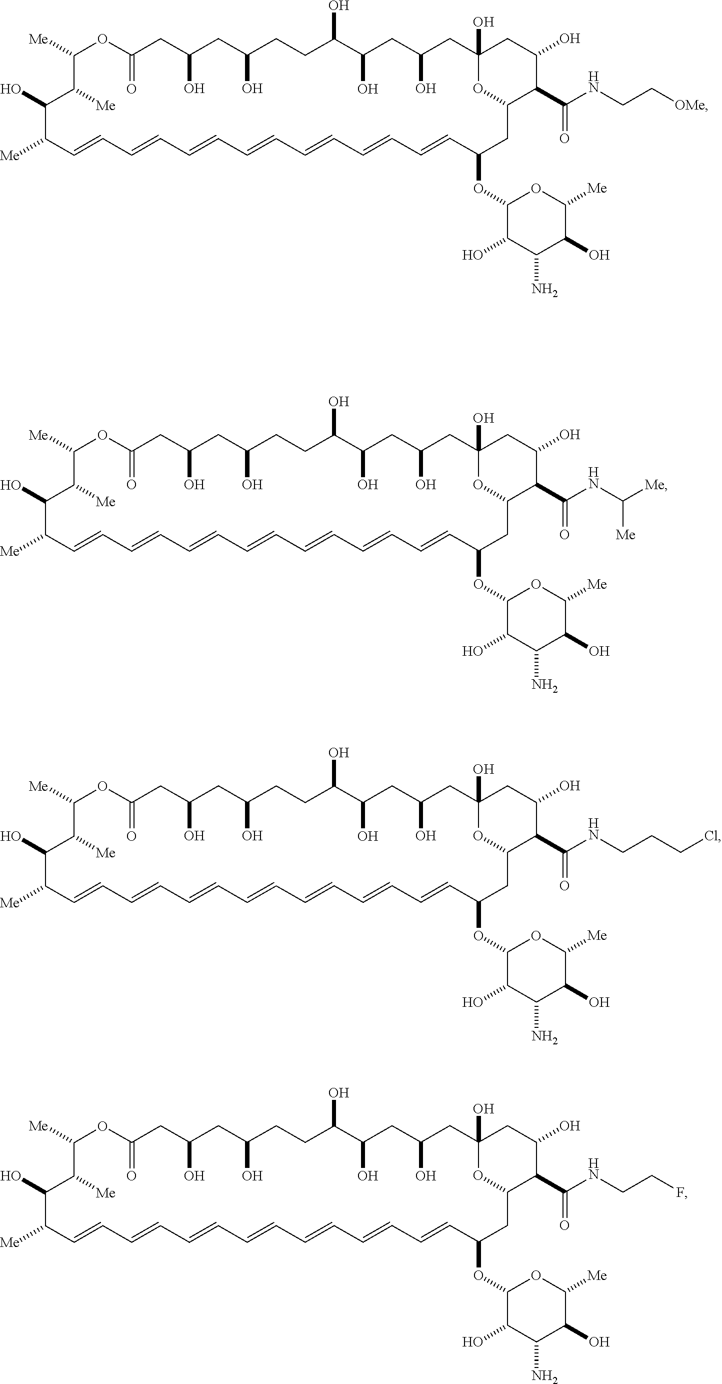

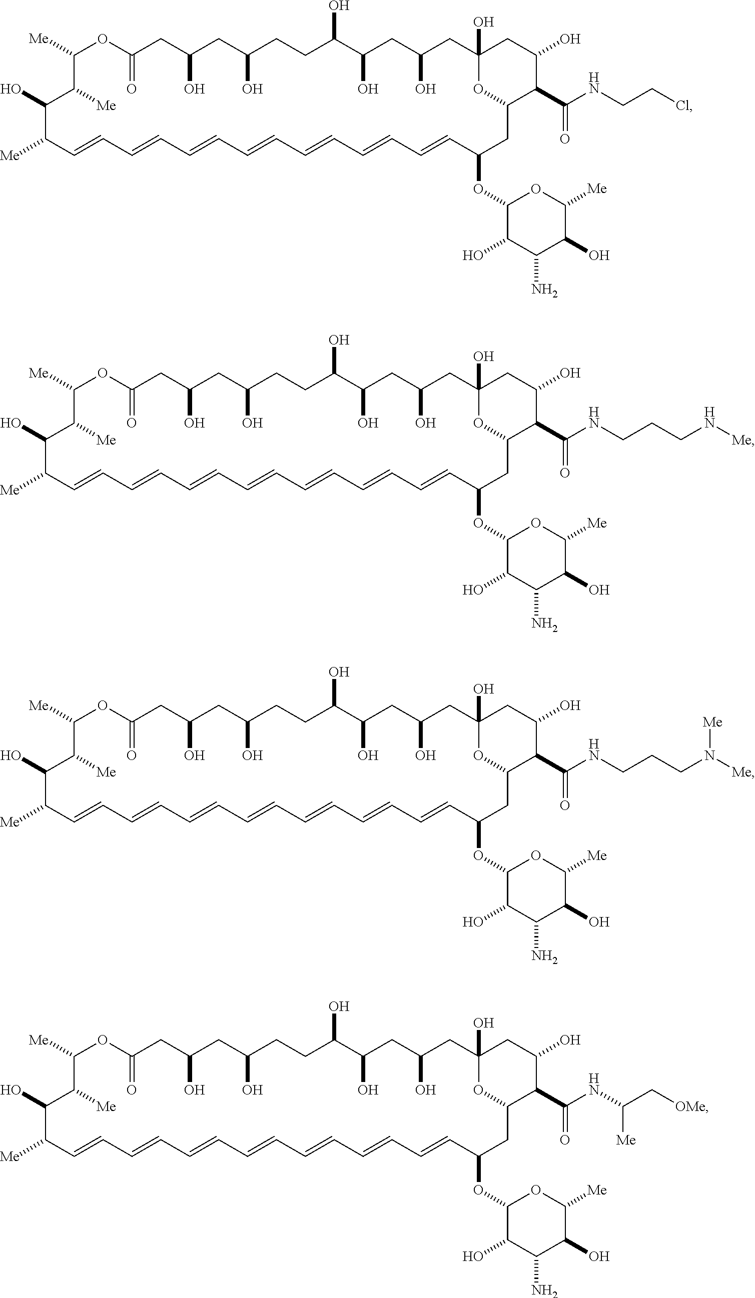

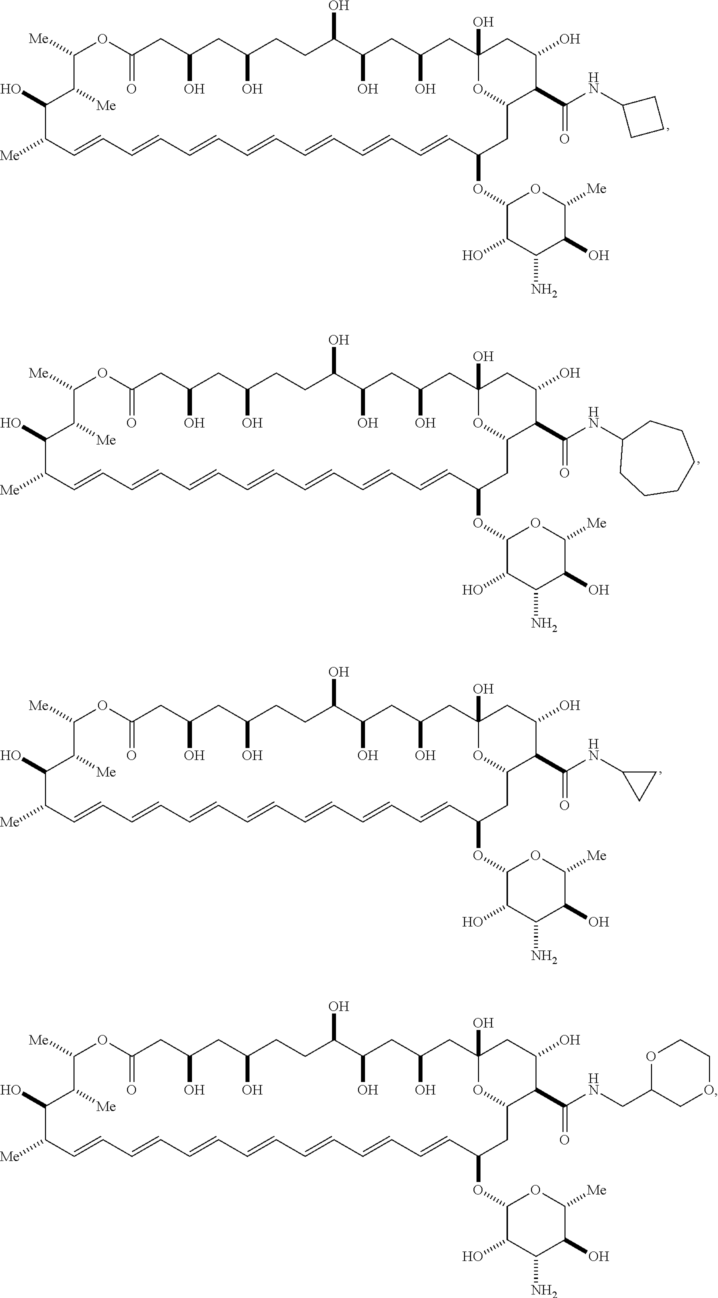

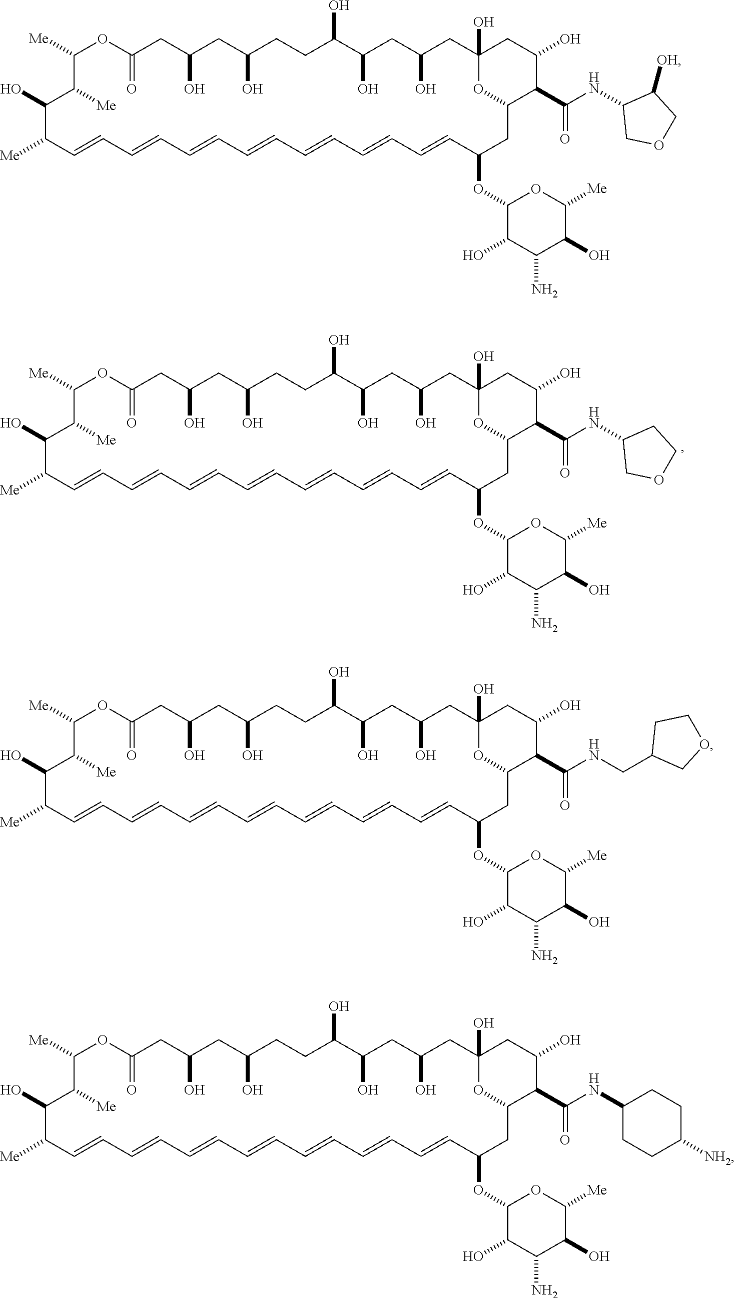

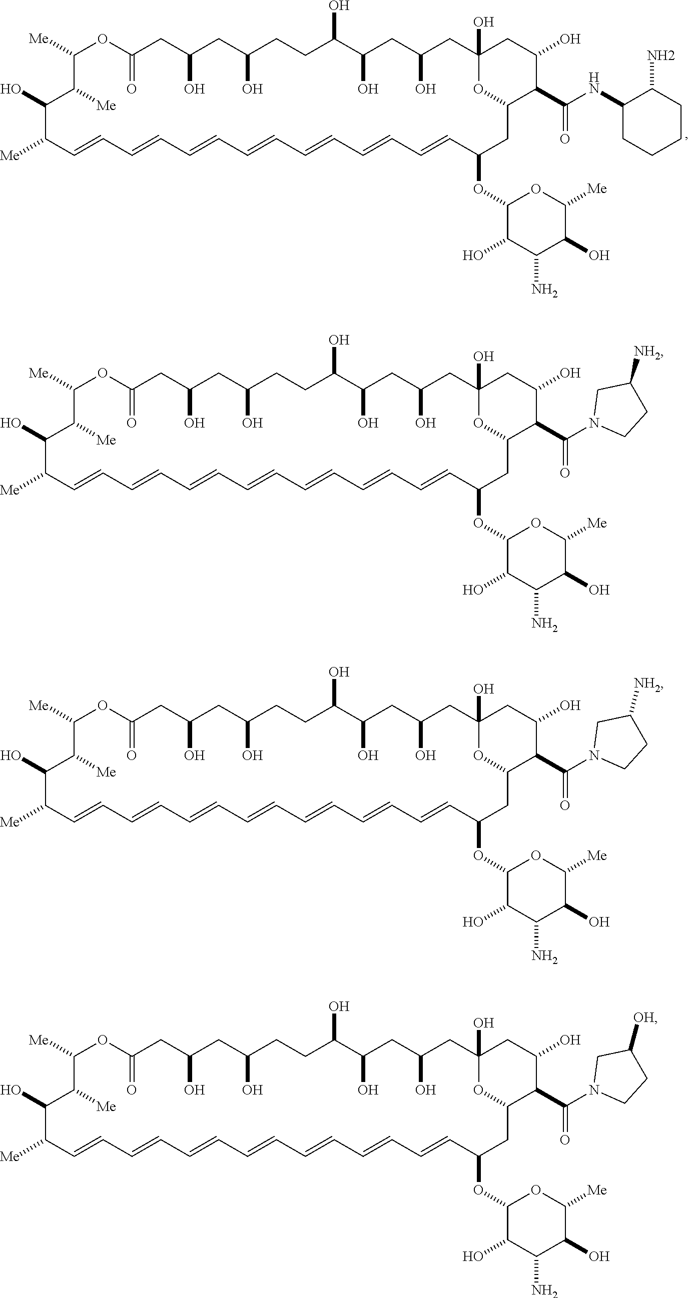

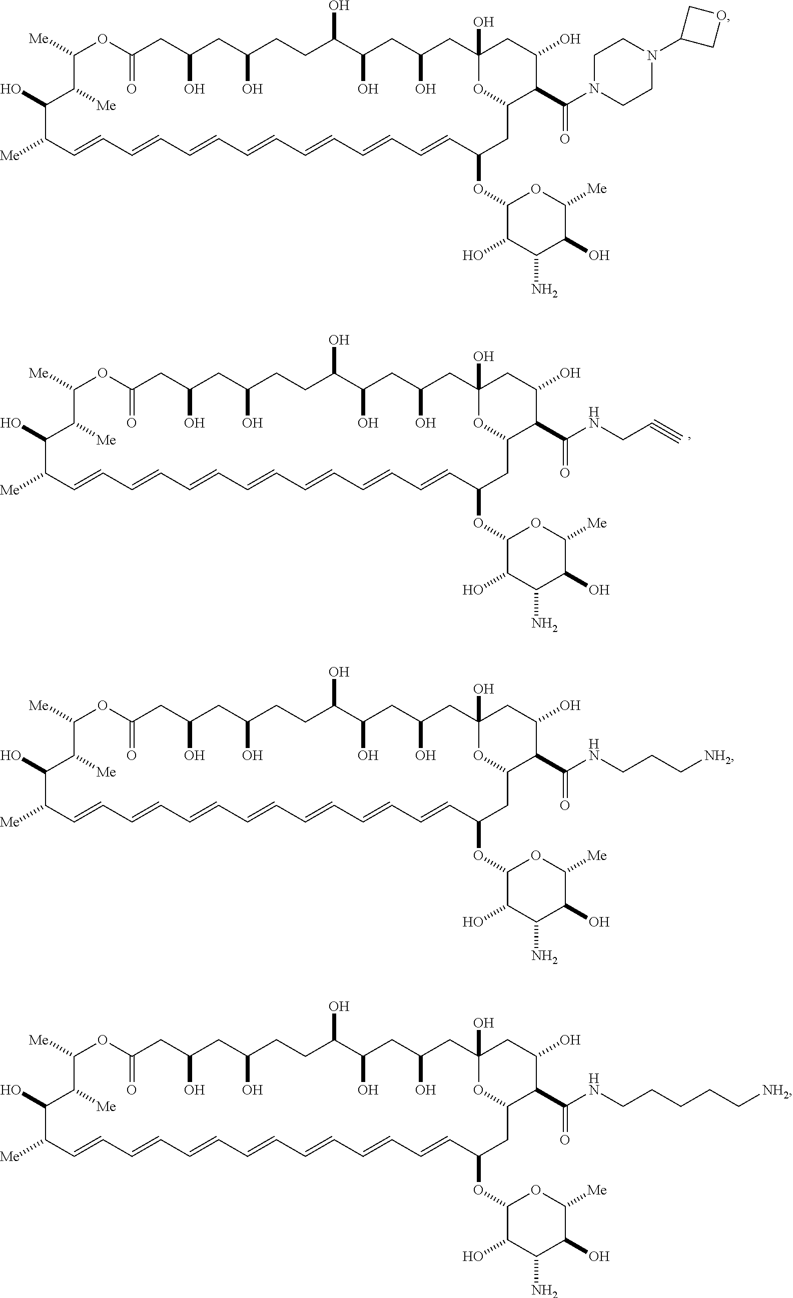

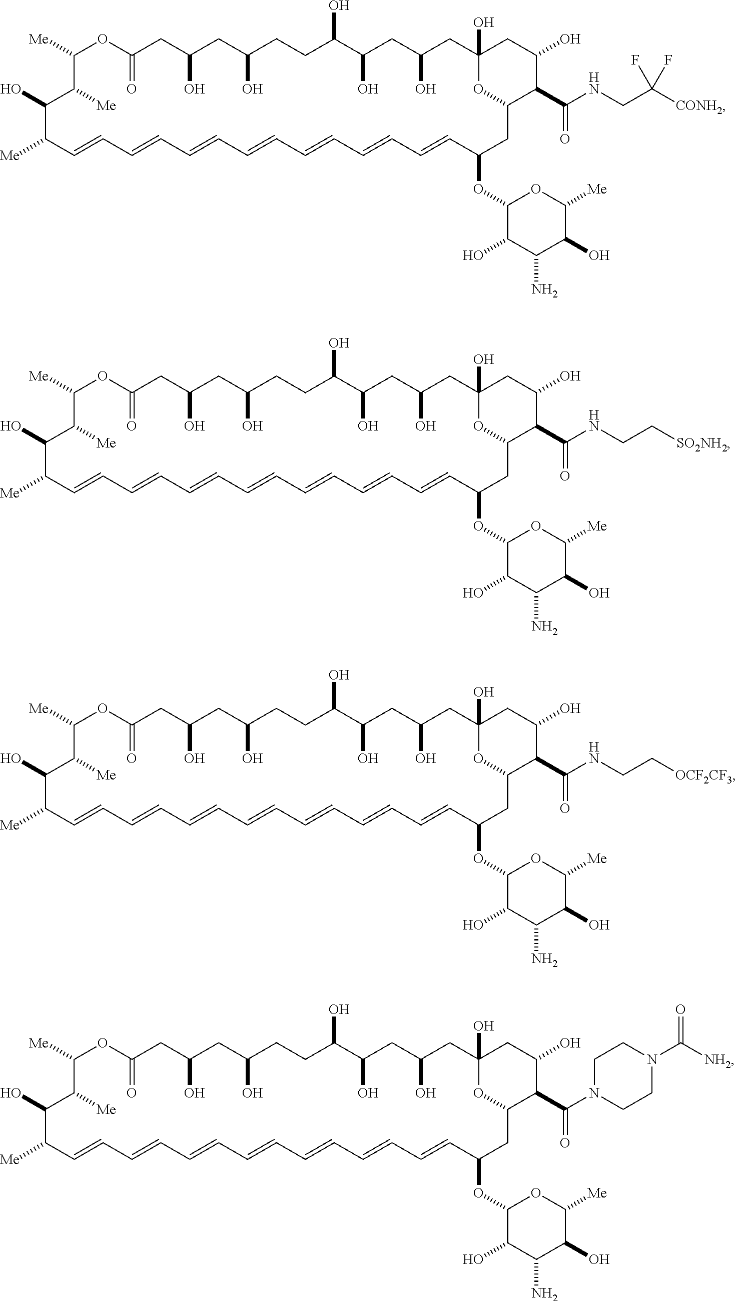

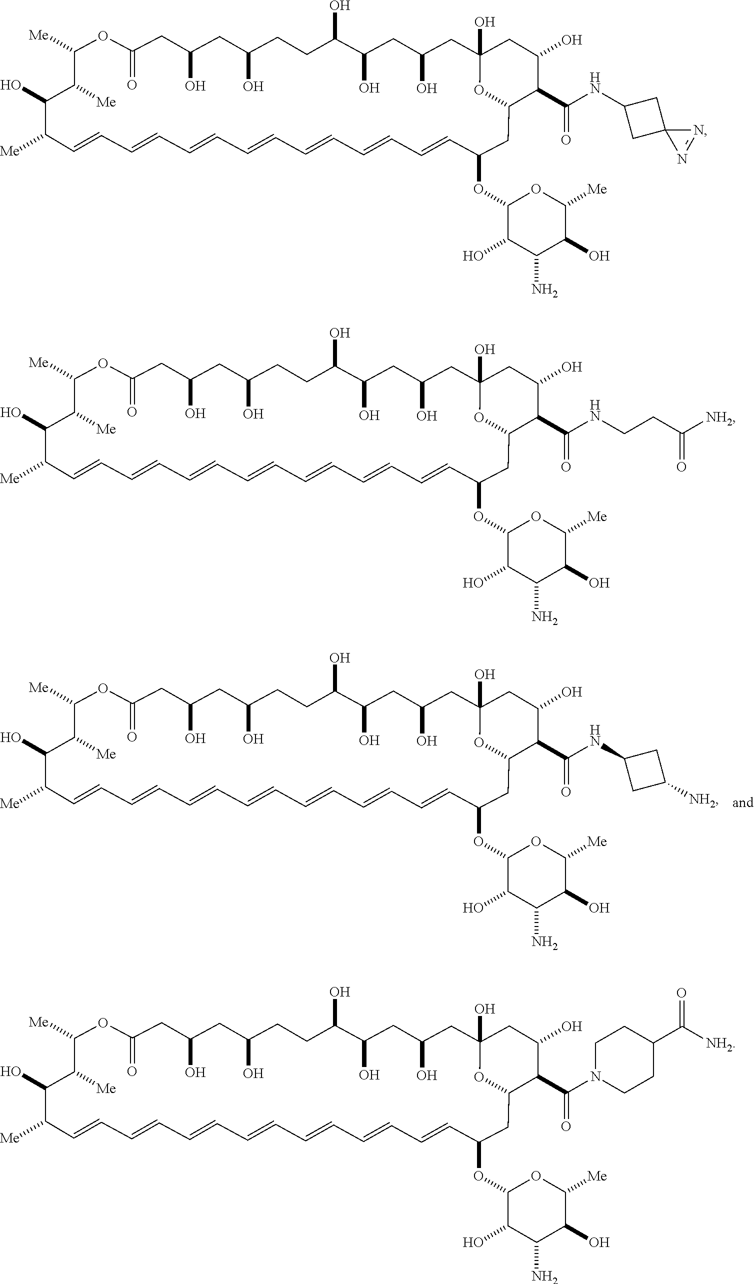

In further embodiments, the compound is selected from the group consisting of:

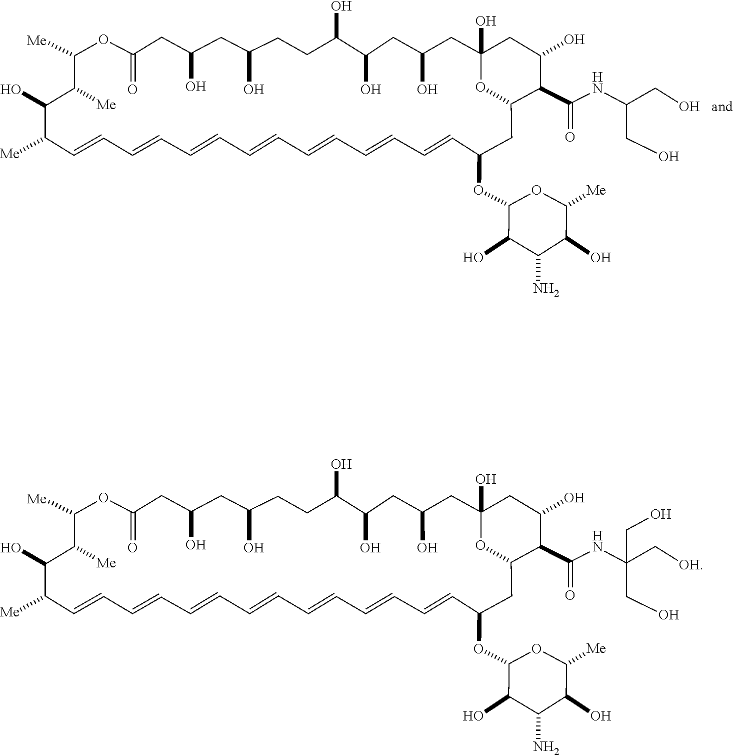

For example, in some embodiments, the compound is:

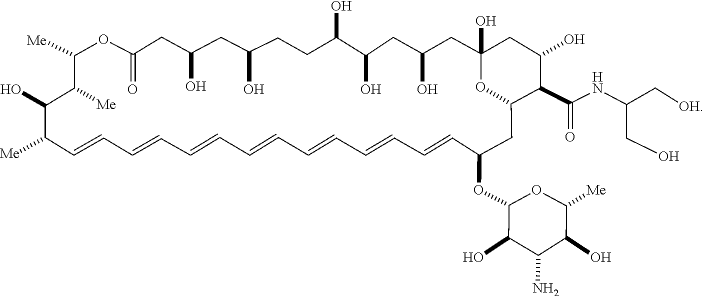

Alternatively, the compound may be:

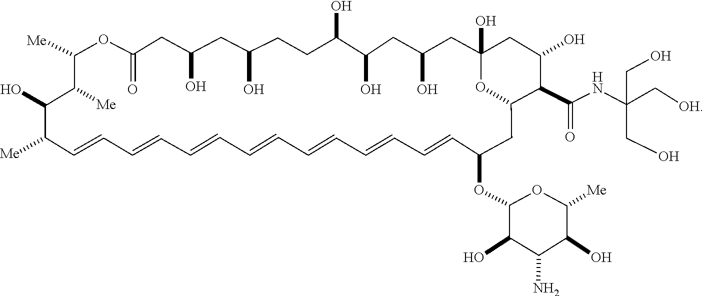

In certain embodiments, the compound is in the form of a pharmaceutically acceptable salt. For example, in certain preferred embodiments, the compound is:

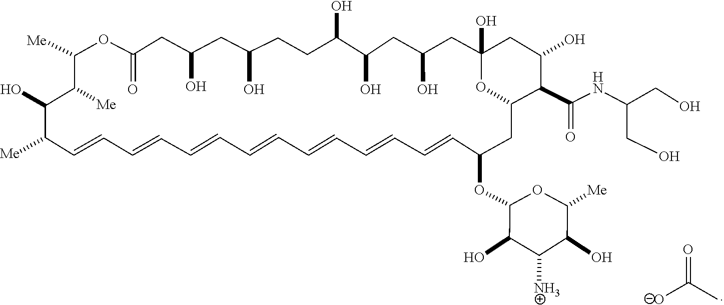

In other such embodiments, the compound is:

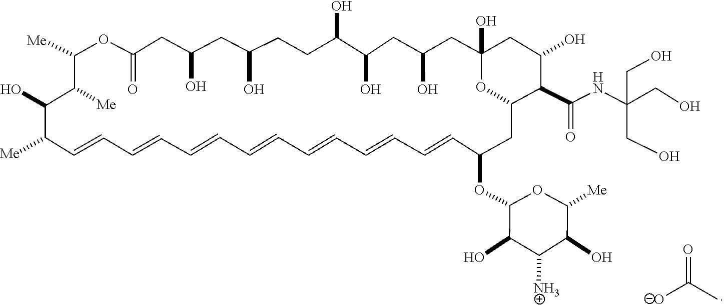

These and other Amphotericin B derivatives, and the synthetic routes and experimental procedures for making these compounds, are disclosed in, e.g., WO 2015/175875, WO 2021/026520, and WO 2022/035752, which publications are incorporated herein by reference.

In certain embodiments, each occurrence of n is independently selected from 1-9, from 2-8, from 3-7, or from 4-6. In certain preferred embodiments, each occurrence of n is 5.

In certain embodiments, m is selected from 20-60, from 30-50, or from 40-50. In certain preferred embodiments, m is 44.

In certain embodiments, the lipid polymer excipient forms micelles in aqueous solution.

In certain embodiments, the composition further comprises an agent for controlling plasma osmolality.

In certain embodiments, the composition further comprises an agent for controlling pH.

In certain embodiments, the composition further comprises an agent for controlling oxidation.

In certain embodiments, the molar ratio of the lipid polymer excipient to the compound is from about 1:1 to about 10:1, from about 1:1 to about 5:1, from about 2:1 to about 4:1, or about 3:1.

In certain embodiments, the lipid polymer excipient is distearoyl-rac-glycerol-polyethylene glycol-2000 (referred to herein as DSG-PEG-2000). In other embodiments, the lipid polymer excipient is 1,2-dimyristoyl-rac-glycero-3-methoxypolyethylene glycol-2000 (alternatively referred to as DMG-PEG-2000 or, for the specific positional isomer 1,2-DMG-PEG-2000). In certain embodiments, DMG-PEG-2000 is a mixture of two positional isomers, 1,2-DMG-PEG-2000 and 1,3-DMG-PEG-2000.

In certain embodiments, the composition comprises, consists essentially of, or consists of:
(i) a lipid polymer excipient having the structure of formula (X);

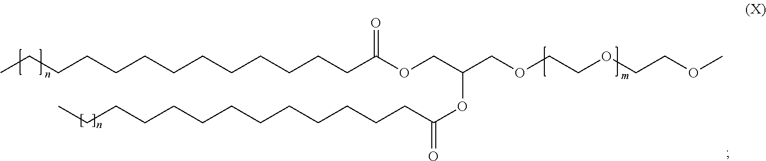

wherein n is 5; and
m is 44; and
(ii) the compound represented by:

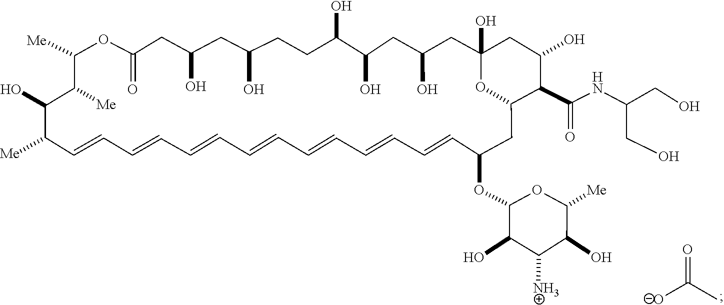

wherein the lipid polymer excipient and the compound are in a molar ratio of about 3:1.

In certain embodiments, the antifungal potency of the composition is greater than the antifungal potency of the compound alone.

In certain embodiments, the in vitro antifungal potency of the composition is higher than the in vitro antifungal potency of the compound alone.

In further embodiments, the in vivo antifungal potency of the composition is higher than the in vivo antifungal potency of the compound alone.

In certain embodiments, the in vivo half-life of the composition is longer than the in vivo half-life of the compound alone.

In certain embodiments, the composition is a slow-release composition.

In certain embodiments, the composition is an intravenous dosage form.

In certain embodiments, the composition further comprises a pharmaceutically acceptable carrier. The term “pharmaceutically acceptable carrier” means one or more compatible solid or liquid filler, diluent, or encapsulating substances which are suitable for administration to a human or other vertebrate animal. The term “carrier” denotes an organic or inorganic ingredient, natural or synthetic, with which the active ingredient is combined to facilitate the administration. The components of the compositions also are capable of being commingled in a manner such that there is no interaction which would substantially impair the desired pharmaceutical efficacy.

The foregoing embodiments of pharmaceutical compositions of the invention are meant to be exemplary and are not limiting.

Also provided is a method for making such pharmaceutical compositions. The method comprises placing a compound of the invention, or a pharmaceutically acceptable salt thereof, in a pharmaceutically acceptable carrier

### Methods of the Invention

The present invention provides a method of treating a fungal infection, comprising administering to a subject in need thereof a therapeutically effective amount of the composition of the present invention, thereby treating the fungal infection. In certain such embodiments, the composition is administered intravenously.

In certain embodiments, the subject is a mammal; or a primate, a canine, a feline, or a bovine; or a human; or a human.

The present invention also provides a use of a composition of the invention in the manufacture of a medicament for treating a fungal infection. In certain such embodiments, the medicament is an intravenous dosage form.

The present invention also provides a composition for use in treating a fungal infection.

In certain embodiments, administration of the composition delivers a dose of 0.01 mg to 10 mg of the compound (e.g., AmB, C2′epiAmB, the compound of formula (I), or the compound of formula (II)).

In certain embodiments, e.g., wherein the composition is a slow-release composition, administration of the composition delivers a daily dose of 0.01 mg to 10 mg of the compound.

In certain embodiments, e.g., wherein the composition is a slow-release composition, the composition is administered once every six months, once every five months, once every four months, once every three months, once every two months, once a month, twice a month, once every two weeks, once a week, twice a week, or three times a week.

In certain embodiments, the composition is an intravenous dosage form.

Compositions of the invention are useful for inhibiting growth of fungi and yeast, including, in particular, fungi and yeast of clinical significance as pathogens. Compositions of the invention are useful in methods of treating fungal and yeast infections, including, in particular, systemic fungal and yeast infections. Compositions of the invention are also useful in the manufacture of medicaments for treating fungal and yeast infections, including, in particular, systemic fungal and yeast infections. The invention further provides the use of compositions of the invention for the treatment of fungal and yeast infections, including, in particular, systemic fungal and yeast infections.

In certain embodiments, the composition is administered intravenously.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modem Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₈ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”).

In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C2-6 alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”).

In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and/or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and lor 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and lor 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “alkylene,” “alkenylene,” “alkynylene,” “heteroalkylene,” “heteroalkenylene,” and “heteroalkynylene,” refer to a divalent radical of an alkyl, alkenyl, alkynyl group, heteroalkyl, heteroalkenyl, and heteroalkynyl group respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon divalent chain. “Alkylene,” “alkenylene,” “alkynylene,” “heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl. In certain embodiments, an aryl group substituted with one or more of groups selected from halo, C₁₋₈ alkyl, C₁₋₈ haloalkyl, cyano, hydroxy, C₁₋₈ alkoxy, and amino.

Examples of representative substituted aryls include the following

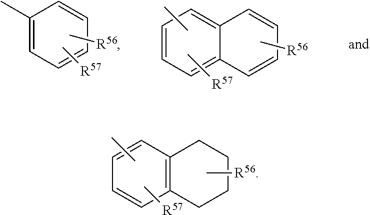

wherein one of R⁵⁶ and R⁵⁷ may be hydrogen and at least one of R⁵⁶ and R⁵⁷ is each independently selected from C₁₋₈ alkyl, C₁₋₈ haloalkyl, 4- to 10-membered heterocyclyl, alkanoyl, C₁₋₈ alkoxy, heteroaryloxy, alkylamino, arylamino, heteroarylamino, NR⁵⁸COR⁵⁹, NR⁵⁸SOR⁵⁹ NR⁵⁸SO₂R⁵⁹, COOalkyl, COOaryl, CONR⁵⁸R⁵⁹, CONR⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁₋₈ alkyl, C₁₋₄ haloalkyl, C₃₋₁₀ carbocyclyl, 4- to 10-membered heterocyclyl, C₆₋₁₀ aryl, substituted C₆₋₁₀ aryl, 5-10 membered heteroaryl, or substituted 5- to 10-membered heteroaryl.

Other representative aryl groups having a fused heterocyclyl group include the following:

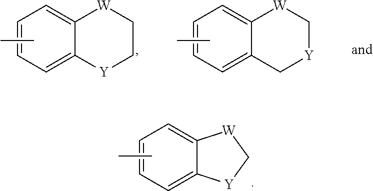

wherein each W is selected from C(R⁶⁶)₂, NR⁶⁶, O, and S; and each Y is selected from carbonyl, NR⁶⁶, O and S; and R⁶⁶ is independently hydrogen, C₁₋₈ alkyl, C₃₋₁₀ carbocyclyl, 4- to 10-membered heterocyclyl, C₆₋₁₀ aryl, and 5- to 10-membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5- to 10-membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5- to 10-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In some embodiments, a heteroaryl group is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In some embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In some embodiments, the 5-6 membered heteroaryl has 1-3 ring heteroatoms selected from nitrogen, oxygen, and sulfur. In some embodiments, the 5- to 6-membered heteroaryl has 1-2 ring heteroatoms selected from nitrogen, oxygen, and sulfur. In some embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

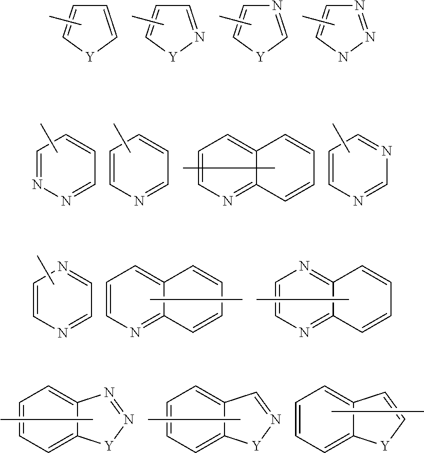

wherein each Y is selected from carbonyl, N, NR⁶⁵, O, and S; and R⁶⁵ is independently hydrogen, C₁₋₈ alkyl, C₃₋₁₀ carbocyclyl, 4-10 membered heterocyclyl, C₆₋₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Examples of C₅₋₆ carbocyclyl groups include cyclopentyl (C₅) and cyclohexyl (C₅). Examples of C₃₋₆ carbocyclyl groups include the aforementioned C₅₋₆ carbocyclyl groups as well as cyclopropyl (C₃) and cyclobutyl (C₄). Examples of C₃₋₈ carbocyclyl groups include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3- to 10-membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 10-membered heterocyclyl. In certain embodiments, the heterocyclyl group is substituted 3- to 10-membered heterocyclyl.

In some embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In some embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In some embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In some embodiments, the 5-6 membered heterocyclyl has 1-3 ring heteroatoms selected from nitrogen, oxygen, and sulfur. In some embodiments, the 5- to 6-membered heterocyclyl has 1-2 ring heteroatoms selected from nitrogen, oxygen, and sulfur. In some embodiments, the 5- to 6-membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

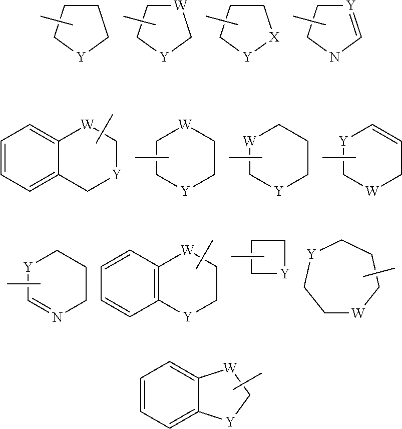

wherein each W is selected from CR⁶⁷, C(R⁶⁷)₂, NR⁶⁷, O, and S; and each Y is selected from NR⁶⁷, O, and S; and R⁶⁷ is independently hydrogen, C₁₋₈ alkyl, C₃₋₁₀ carbocyclyl, 4- to 10-membered heterocyclyl, C₆₋₁₀ aryl, 5- to 10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, carbocyclyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Hetero” when used to describe a compound or a group present on a compound means that one or more carbon atoms in the compound or group have been replaced by a nitrogen, oxygen or sulfur heteroatom. Hetero may be applied to any of the hydrocarbyl groups described above such as alkyl, e.g., heteroalkyl, carbocyclyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁₋₈ alkyl, —C(O)—(CH₂)ₜ(C₆₋₁₀ aryl), —C(O)—(CH₂)ₜ(5- to 10-membered heteroaryl), —C(O)—(CH₂)ₜ(C₃₋₁₀ carbocyclyl), and —C(O)—(CH₂)ₜ(4- to 10-membered heterocyclyl), wherein t is an integer from 0 to 4. In certain embodiments, R is C₁₋₈ alkyl, substituted with halo or hydroxy; or C₃₋₁₀ carbocyclyl, 4- to 10-membered heterocyclyl, C₆₋₁₀ aryl, arylalkyl, 5- to 10-membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Acylamino” refers to a radical —NR²²C(O)R²³, where each instance of R²² and R²³ is independently hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁₋₈ alkyl, —NR²⁴C(O)—(CH₂)ₜ(C₆₋₁₀ aryl), —NR²⁴C(O)—(CH₂)ₜ(5- to 10-membered heteroaryl), —NR²⁴C(O)—(CH₂)ₜ(C₃₋₁₀ carbocyclyl), and —NR²⁴C(O)—(CH₂)ₜ(4- to 10-membered heterocyclyl), wherein t is an integer from 0 to 4, and each R²⁴ independently represents H or C₁₋₈ alkyl. In certain embodiments, R²⁵ is H, C₁₋₈ alkyl, substituted with halo or hydroxy; C₃₋₁₀ carbocyclyl, 4- to 10-membered heterocyclyl, C₆₋₁₀ aryl, arylalkyl, 5-10 membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; and R²⁶ is H, C₁₋₈ alkyl, substituted with halo or hydroxy; C₃₋₁₀ carbocyclyl, 4-10 membered heterocyclyl, C₆₋₁₀ aryl, arylalkyl, 5-10 membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxyl; provided at least one of R²⁵ and R²⁶ is other than H.

“Acyloxy” refers to a radical —OC(O)R²⁷, where R²⁷ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁₋₈ alkyl, substituted with halo or hydroxy; C₃₋₁₀ carbocyclyl, 4- to 10-membered heterocyclyl, C₆₋₁₀ aryl, arylalkyl, 5- to 10-membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Alkoxy” refers to the group —OR²⁹ where R²⁹ is substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆₋₁₀ aryl, aryloxy, carboxyl, cyano, C₃₋₁₀ carbocyclyl, 3- to 10-membered heterocyclyl, halogen, 5- to 10-membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆₋₁₀ aryl), —O—(CH₂)ₜ(5- to 10-membered heteroaryl), —O—(CH₂)ₜ(C₃₋₁₀ carbocyclyl), and —O—(CH₂)ₜ(4- to 10-membered heterocyclyl), wherein t is an integer from 0 to 4 and any aryl, heteroaryl, carbocyclyl or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³¹ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments, each R³⁸ is independently selected from hydrogen, C₁₋₈ alkyl, C₃₋₈ alkenyl, C₃₋₈ alkynyl, C₆₋₁₀ aryl, 5- to 10-membered heteroaryl, 4- to 10-membered heterocyclyl, or C₃₋₁₀ carbocyclyl; or C₁₋₈ alkyl, substituted with halo or hydroxy; C₃₋₈ alkenyl, substituted with halo or hydroxy; C₃₋₈ alkynyl, substituted with halo or hydroxy, or —(CH₂)ₜ(C₆₋₁₀ aryl), —(CH₂)ₜ(5- to 10-membered heteroaryl), —(CH₂)ₜ(C₃₋₁₀ carbocyclyl), or —(CH₂)ₜ(4- to 10-membered heterocyclyl), wherein t is an integer between 0 and 8, each of which is substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; or both R groups are joined to form an alkylene group.

Exemplary “substituted amino” groups include, but are not limited to, —NR³⁹—C₁₋₈ alkyl, —NR³⁹—(CH₂)ₜ(C₆₋₁₀ aryl), —NR³⁹—(CH₂)ₜ(5-10 membered heteroaryl), —NR³⁹—(CH₂)ₜ(C₃₋₁₀ carbocyclyl), and —NR³⁹—(CH₂)ₜ(4-10 membered heterocyclyl), wherein t is an integer from 0 to 4, for instance 1 or 2, each R³⁹ independently represents H or C₁₋₈ alkyl; and any alkyl groups present, may themselves be substituted by halo, substituted or unsubstituted amino, or hydroxy; and any aryl, heteroaryl, carbocyclyl, or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R⁶² is not a hydrogen. In certain embodiments, R⁶² is selected from H, C₁₋₈ alkyl, C₃₋₁₀ carbocyclyl, 4- to 10-membered heterocyclyl, C₆₋₁₀ aryl, aralkyl, 5- to 10-membered heteroaryl, and heteroaralkyl; or C₁₋₈ alkyl substituted with halo or hydroxy; or C3-10 carbocyclyl, 4- to 10-membered heterocyclyl, C₆₋₁₀ aryl, aralkyl, 5- to 10-membered heteroaryl, or heteroaralkyl, each of which is substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; provided that at least one R⁶² is other than H.

Exemplary “substituted carbamoyl” groups include, but are not limited to, —C(O)NR⁶⁴—C₁₋₈ alkyl, —C(O)NR⁶⁴—(CH₂)ₜ(C₆₋₁₀ aryl), —C(O)N⁶⁴—(CH₂)ₜ(5- to 10-membered heteroaryl), —C(O)NR⁶⁴—(CH₂)ₜ(C₃₋₁₀ carbocyclyl), and —C(O)NR⁶⁴—(CH₂)ₜ(4- to 10-membered heterocyclyl), wherein t is an integer from 0 to 4, each R⁶⁴ independently represents H or C₁₋₈ alkyl and any aryl, heteroaryl, carbocyclyl or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Carbocyclylalkyl” refers to an alkyl radical in which the alkyl group is substituted with a carbocyclyl group. Typical carbocyclylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethylene” refers to substituted or unsubstituted —(C—C)—.

“Ethenyl” refers to substituted or unsubstituted —(C═C)—.

“Ethynyl” refers to —(C═C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})—TXk—N(OR^{cc})R^{bb}, —SH, —SR^{aa}, —SSR^{CC}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{CC})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂—NR^{bb}P(═O)(NR^{bb})₂, —P(R^{CC})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, —NNR^{bb}C(═O)R^{aa}, —NNR^{bb}C(═O)OR^{aa}, —NNR^{bb}S(═O)₂R^{aa}, ═NR^{bb}, or ═NOR^{CC};
each instance of R^{aa} is, independently, selected from C₁₋₁₀ alkyl, C₁₋₁₀ perhaloalkyl, C₂₋₁₀ alkenyl, C₂₋₁₀ alkynyl, C3-10 carbocyclyl, 3- to 14-membered heterocyclyl, C₆₋₁₄ aryl, and 5- to 14-membered heteroaryl, or two R^{aa} groups are joined to form a 3-14 membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{CC})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ee}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ee}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3- to 10-membered heterocyclyl, C₆₋₁₀ aryl, 5- to 10-membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 3- to 10-membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen C₁₋₆ alkyl, C₁₋₆ perhaloalkyl, C₂₋₆ alkenyl, C₂₋₆ alkynyl, C₃₋₁₀ carbocyclyl, 3- to 10-membered heterocyclyl, C₆₋₁₀ aryl and 5- to 10-membered heteroaryl, or two R^{ff} groups are joined to form a 3-14 membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₂⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, 5- to 10-membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄²-sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxy late ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —OR^{aa}, —N(R^{CC})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})0R^{aa}, —C(═NR^{CC})N(R^{CC})₂, —SO₂N(R^{CC})₂, —SO₂R^{Cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{CC})₂, —P(═O)(NR^{CC})₂ C₁₋₁₀ alkyl, C₁₋₁₀ perhaloalkyl, C₂₋₁₀ alkenyl, C₂₋₁₀ alkynyl, C₃₋₁₀ carbocyclyl, 3- to 14-membered heterocyclyl, C₆₋₁₄ aryl, and 5- to 14-membered heteroaryl, or two R^{cc} groups attached to a nitrogen atom are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and claims. The invention is not intended to be limited in any manner by the above exemplary listing of substituents.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

“Pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁₋₈ alkyl, C₂₋₈ alkenyl, C₂₋₈ alkynyl, aryl, C₇₋₁₂ substituted aryl, and C₇₋₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

As used herein, a “subject” refers to a living mammal. In various embodiments a subject is a non-human mammal, including, without limitation, a mouse, rat, hamster, guinea pig, rabbit, sheep, goat, cat, dog, pig, horse, cow, or non-human primate. In one embodiment a subject is a human.

As used herein, a “subject having a fungal infection” refers to a subject that exhibits at least one objective manifestation of a fungal infection. In one embodiment a subject having a fungal infection is a subject that has been diagnosed as having a fungal infection and is in need of treatment thereof. Methods of diagnosing a fungal infection are well known and need not be described here in any detail.

As used herein, a “subject having a yeast infection” refers to a subject that exhibits at least one objective manifestation of a yeast infection. In one embodiment a subject having a yeast infection is a subject that has been diagnosed as having a yeast infection and is in need of treatment thereof. Methods of diagnosing a yeast infection are well known and need not be described here in any detail.

As used herein, the phrase “effective amount” refers to any amount that is sufficient to achieve a desired biological effect.

As used herein, the phrase “therapeutically effective amount” refers to an amount that is sufficient to achieve a desired therapeutic effect, e.g., to treat a fungal or yeast infection.

For any compound described herein, a therapeutically effective amount can, in general, be initially determined from in vitro studies, animal models, or both in vitro studies and animal models. In vitro methods are well known and can include determination of minimum inhibitory concentration (MIC), minimum fungicidal concentration (MFC), concentration at which growth is inhibited by 50 percent (IC₅₀), concentration at which growth is inhibited by 90 percent (IC₉₀), and the like. A therapeutically effective amount can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described herein and other methods as are well-known in the art is well within the capabilities of the ordinarily skilled artisan.

For any compound described herein, a therapeutically effective amount for use in human subjects can be initially determined from in vitro studies, animal models, or both in vitro studies and animal models. A therapeutically effective amount for use in human subjects can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, or 95 percent (%) compared to control.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease. For example, in one embodiment the terms “treating” and “treat” refer to performing an intervention that results in (a) inhibiting a fungal infection, e.g., slowing or arresting its development; or (b) relieving or ameliorating a fungal infection, e.g., causing regression of the fungal infection.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset).

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

A “fungal infection” as used herein refers to an infection in or of a subject with a fungus as defined herein. In one embodiment the term “fungal infection” includes a yeast infection. A “yeast infection” as used herein refers to an infection in or of a subject with a yeast as defined herein.

“Active ingredient”, “therapeutically active ingredient”, “active agent”, “drug” or “drug substance” as used herein means the active ingredient of a pharmaceutical, also known as an active pharmaceutical ingredient (API).

“Drug Loading” as used herein refers to the percentage of active ingredient(s) on a mass basis in the total mass of the formulation.

The term “about” refers to variations in numerical values typically encountered by one of skill in the art of respirable formulations, including variations of plus or minus 0.1%, 0.5%, 1%, 2%, 3%, 4%, 5%, 6%, 7%, 8%, 9%, or 10% of a numerical value described herein.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Unless otherwise stated, or clear from the context, numerical ranges include both the endpoints and any value between.

### Packaged Pharmaceutical Products

In yet further aspects, provided herein are packaged pharmaceutical products, comprising a composition of the invention.

In certain embodiments, the composition is a slow-release composition.

In certain embodiments, the composition is an intravenous dosage form.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In some embodiments, intravenous administration of a compound of the invention may typically be from 0.1 mg/kg/day to 20 mg/kg/day. Intravenous dosing thus may be similar to, or advantageously, may exceed maximal tolerated doses of AmB. Intravenous dosing also may be similar to, or advantageously, may exceed maximal tolerated daily doses of AmB. Intravenous dosing also may be similar to, or advantageously, may exceed maximal tolerated cumulative doses of AmB.

Intravenous dosing also may be similar to, or advantageously, may exceed maximal recommended doses of AmB. Intravenous dosing also may be similar to, or advantageously, may exceed maximal recommended daily doses of AmB. Intravenous dosing also may be similar to, or advantageously, may exceed maximal recommended cumulative doses of AmB.

For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Amphotericin B is commercially available in a number of formulations, including deoxycholate-based (sometimes referred to as desoxycholate-based) formulations and lipid-based (including liposomal) formulations.

For intravenous administration, the active pharmaceutical ingredient may be stabilized in micelles. In certain embodiments, the micelles are formed of block copolymers. In further embodiments, the micelles are formed of multiple components (e.g., a block copolymer and a deoxycholate compound) and can thus be referred to as “mixed micelles.”

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. Formulations for injection may alternatively be formulated for sustained or slow release. The compositions may take such forms as suspensions, solutions, or emulsions in oily or aqueous vehicles, and may contain formulatory agents such as suspending, stabilizing and/or dispersing agents. Stabilizing agents include, for example, compounds capable of forming micelles.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions or improve the half-life of a compound in solution.

Alternatively, the active compounds may be in powder form for constitution with a suitable vehicle, e.g., sterile pyrogen-free water, before use.

In addition to the formulations described above, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally at least one additional therapeutic agent included in a pharmaceutically acceptable carrier.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

A therapeutic agent such as a compound described herein may be provided in a micellar formulation. Micellar formulations may be prepared using standard techniques whereby the a polymeric component (e.g., the lipid polymer excipient) and an active pharmaceutical ingredient are thoroughly mixed or intermingled. Mechanical mixing procedures may be employed to achieve a thorough blending of the ingredients of the composition.

Injection devices such as syringes may be prepared so as to contain micellar formulations by using any technique whereby the composition is placed within the injection device in a manner that the composition becomes injectable by the device. For example, a composition of this invention may be placed within the barrel of a syringe by mechanical means or extrusion.

Compositions of this invention may be stored for substantial lengths of time. In certain embodiments, the composition may be stored at room temperature, or at a temperature below that of room temperature.

Compositions of this invention may be placed in sterile containers for subsequent pharmaceutical formulation. Such a container may be a sealed vial which preferably will contain sufficient space for the subsequent addition of an aqueous, physiologically acceptable carrier. Thus, the compositions of this invention may be employed for production of drug containing micelles within the aforementioned container after introduction of the aqueous carrier. Dissolution of the composition in the carrier with concomitant formation of drug containing micelles may be accelerated by agitation (e.g., shaking) or without agitation over time.

Methods for administration of compositions according to this invention may be done according to methods known in the art. Methodologies for injection of such compositions or solutions at a selected site within the body of a patient may be selected and performed by a medical professional.

In certain embodiments, the lipid polymer excipient in the compositions of the invention is a biocompatible micelle forming polymer. Exemplary biocompatible micelle forming polymers include polymers known in the art, such as those described in WO 01/87345. In certain embodiments, one or more micelle forming polymers in compositions of this invention will be a diblock copolymer suitable for formation of micelles as taught in the art or as specifically described herein.

Hydrophobic portions of such diblock copolymers may comprise one or more hydrophobic polymers, such as polyesters, polyanhydrides, polyglycolic acids, polybutrylactones, polyhydroxybutyrates, polylactic acids and polylacaprolactones. The hydrophobic portion of the copolymer may comprise one or more different hydrophobic polymers in random or block orientation. In certain embodiments, the hydrophobic portion of a copolymer will have a molecular weight from about 200 to about 5000.

Hydrophilic portions of micelle forming copolymers that may be used in this invention have a molecular weight of about 750 or greater up to about 8000. In some embodiments, the molecular weight will be in the range of about 1000 or 2000-3000 or 5000. In some embodiments, the hydrophilic portion of the micelle forming copolymer is a polyethylene glycol.

Weight ratios of hydrophobic and hydrophilic components of micelle forming polymers used in this invention may be adjusted to provide for a desired chemistry, manufacturing and controls. For injection, it is preferred that the amount of lipid polymer excipient be such that the resulting mixture or matrix is injectable, as defined herein. The amount of active pharmaceutical ingredient included in the composition will be such as to provide a desired amount of drug loaded micelles, preferably not exceeding an amount that can be sufficiently distributed within the micelle forming composition.

In certain embodiments, the drug-loaded micelles in the compositions of the present invention are freeze-dried after preparation and stored in the dry state. Dry micelles may be reconstituted in a pharmaceutically acceptable carrier such as sterile physiological saline or a sterile dextrose solution, e.g., 5% dextrose, and after thorough hydration, they can be filter sterilized (optionally through a 0.22 m filter) prior to administration.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### EXAMPLES

Having now described the present invention in detail, the same will be more clearly understood by reference to the following examples, which are included herewith for purposes of illustration only and are not intended to be limiting of the invention.

The Amphotericin B derivatives used in the compositions of the invention, and the synthetic routes and experimental procedures for making these compounds, are disclosed in, e.g., WO 2015/175875, WO 2021/026520, and WO 2022/035752, which publications are incorporated herein by reference.

### Example 1: Stability and Plasma Compatibility of AM-2-19

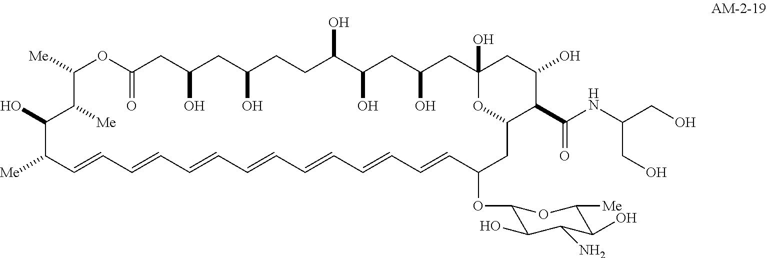

AM-2-19 (and its acetate salt AM-2-19-OAc) is a potent antifungal compound that shows excellent efficacy against numerous fungal pathogens, exhibits a long half-life, and mitigates renal toxicity as compared to AmB and other AmB derivatives. However, AM-2-19 suffers from poor plasma compatibility and solution stability over time.

The in vitro plasma compatibility of AM-2-19 in human plasma is represented below:

| | | | | Human | |
|---|---|---|---|---|---|
| | Final Concentration | Biological | | Precipitate/Pellet or | |
| Ratio | AM-2-19 (mg/mL) | matrix | Solution added | Coagulation Observed | Compatibility |
| 1:1 | 2.50 | Plasma | AM-2-19 | Pellet (Severe) | Not compatible |
| | 1.00 | | | Pellet (Moderate) | Not compatible |
| | 0.50 | | | Pellet (Light) | Not compatible |
| | — | | 5% dextrose | None | compatible |
| | — | | 0.9% Saline | None | compatible |
| 1:0.1 | 0.45 | | AM-2-19 | Pellet (Very Light) | Not compatible |
| | 0.18 | | | None | compatible |
| | 0.09 | | | None | compatible |
| | — | | 5% dextrose | None | compatible |
| | — | | 0.9% Saline | None | compatible |

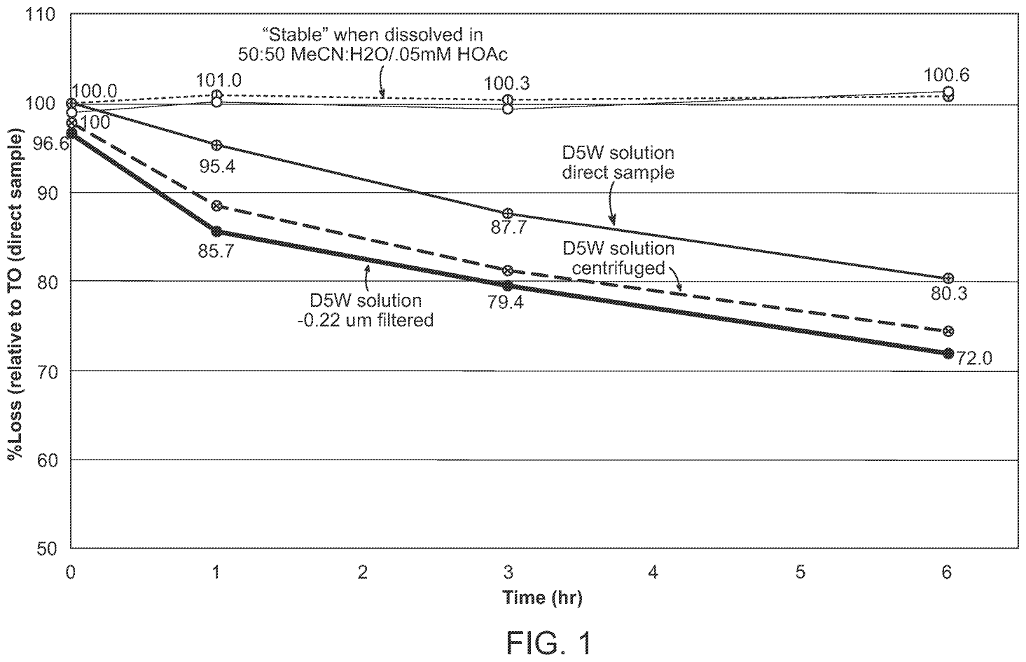

As shown in FIG. 1, AM-2-19 lacks solution stability in IV-compatible solvents such as 5% dextrose in water (D5W).

The following experiments were undertaken to improve plasma compatibility and solution stability of this promising antifungal compound.

### Example 2: Stability and Plasma Compatibility of Micellar Formulations

Several micellar formulations of AM-2-19 OAc were prepared and tested according to the following protocol:
Mix 0.5 mL of formulation solution (2.5 mg/mL) to 0.5 mL of plasma (ITR protocol)
Visually check the solution
Spin it down @1600 g and check for pellet
Spin it down @5000 g and check for pellet
Spin it down @20000 g and check for pellet
Negative Control: AM-2-19-OAc in D5W, saline and water
Positive Control: only D5W, saline and water

Plasma incompatibility is marked by a cloudy mixture once the micellar formulation mixes with plasma. Additionally, the presence of a pellet after centrifugation of the plasma-micellar formulation mixture indicates plasma incompatibility.

The results are shown below.

Micellar formulations of AM-2-19-OAc

| | Visual | | | After plasma | pellet | Pellet | pellet | % conc. | % conc. |
|---|---|---|---|---|---|---|---|---|---|
| Expt | check | Excipient | vehicle | addition | @1600 g | @5000 g | @20K g | @3 h | @24 h |
| 1 | clear | Poloxamer 188 | water | cloudy | Y | | | 98.6 | 75.3 |
| 2 | clear | DSG-PEG 2000 | d5w | clear | N | N | N | 98 | 98 |
| 3 | clear | DSG-PEG 2000 | saline | clear | N | N | N | 97.2 | 92.6 |
| 4 | clear | DSG-PEG 2000 | water | clear | N | N | N | 106.5 | 90 |
| 5 | clear | Chol 600 PEG | d5w | slight cloudy | cloudy | Y | | | |

### Control Formulations

| | | Visual | | | After plasma | pellet | pellet | pellet |
|---|---|---|---|---|---|---|---|---|
| Expt | Sample Name | check | Excipient | vehicle | addition | @1600 g | @5000 g | @20K g |
| 23 | Control D5W | clear | — | d5w | cloudy | Y | | |
| 24 | Control saline | clear | — | saline | cloudy | Y | | |
| 25 | Control water | clear | — | water | cloudy | Y | | |
| 26 | Only D5W | | — | d5w | clear | N | N | N |
| 27 | Only 0.9% Saline | | — | saline | clear | N | N | N |
| 28 | Only water | | — | water | clear | N | N | N |

Formulations of AM-2-19-OAc and DSG-PEG-2000 exhibited plasma compatibility in D5W, saline, and water, whereas none of the formulations of AM-2-19-OAc alone exhibited plasma compatibility.

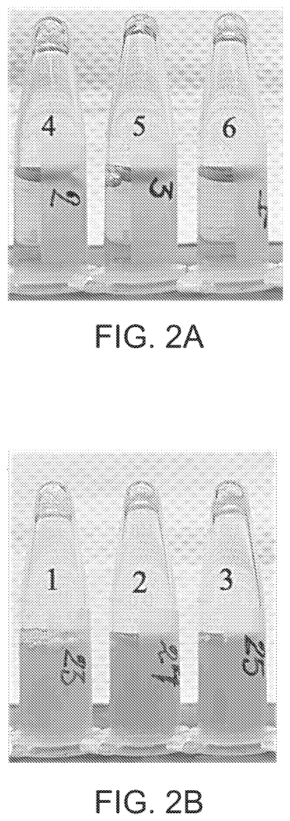

An image of the AM-2-19-OAc and DSG-PEG-2000 formulations (i.e., experiments 2, 3, and 4) after plasma addition is shown in FIG. 2A, and an image of the negative control formulations (i.e., experiments 23, 24, and 25) after plasma addition is shown in FIG. 2B.

### Example 3: Preparation of AM-2-19-FB (Free Base) in 30 mM Acetate in D5W pH 5, DSG-PEG2000

As indicated in Example 1, AM-2-19-OAc suffers from concentration variation over time in D5W solution. Additionally, solutions of AM-2-19-OAc in D5W are incompatible with plasma, hindering an intravenous formulation of the compound.

The inventors hypothesized that the concentration variation might be caused by aggregation and/or adsorption on glass. Similarly, aggregation, promoted by pH and the amphiphilic nature of the compound may have led to plasma incompatability.

The inventors surprisingly discovered that micellar stabilization of the Amphotericin derivative not only significantly improves stability and plasma compatibility, but also led to surprising improvements in potency and half-life, as detailed below.

Note: The AM-2-19-FB:DSG-PEG2000 mol ratio is fixed at 1:3

### Step 1. Placebo Formulation Preparation (30 mM Acetate in D5W, pH 5, DSG-PEG 2000)

Prepare 30 mM acetate in D5W using glacial acetic acid in a beaker
Adjust pH with ION NaOH
Transfer to volumetric flask and QS with D5W
Re-check pH (typically does not change)
Sparge nitrogen through the solution 5-15 min. (depending on volume prepared)
Weigh DSG-PEG2000 into a vial
Weigh required amount of 30 mM acetate in D5W pH 5 into the vial containing DSG-PEG2000
Sonicate for 5 min
Add a stir bar and stir until completely dissolved

### Step 2. AM-2-19-FB Formulation Preparation

Weigh AM-2-19-FB into a vial
Weigh and add the required amount of placebo formulation prepared in Step 1 to the vial
Vortex 10 seconds
Add a stir bar and heat at 50° C. with rapid stirring for 30 min.
Cool to room temperature on ice
Filter through a 0.22 μm PVDF syringe filter

Lower concentrations of AM-2-19-FB may be prepared via serial dilutions of the above formulation using D5W as diluent.

Example for a 2 mg/mL AM-2-19-FB Formulation (Typical Stock Solution Concentration)

| AM-19 | | Purity (specific to | | | |
|---|---|---|---|---|---|
| conc. | | each AM-2-19-FB | Correction | | AM-2-19-Ac |
| (mg/mL) | MW | lot) | Factor (1/Purity) | AM-2-19 M | mg/mL |
| 2.0 | 997.140 | 0.858 | 1.166 | 2.006 × 10⁻³ | 2.331 |

| DSG:PEG | DSG:PEG | DSG-PEG | DSG-PEG | D5W Mean | DSG:PEG in | D5W density |
|---|---|---|---|---|---|---|
| molar ratio | moles | MW | (mg/mL) | Density | 1 mL (g) | (g/mL) |
| 3 | 6.017 × 10⁻³ | 2621.41 | 15.77 | 1.026 | 0.01577 | 1.026 |

Note:
the solubility of AM-2-19-FB in D5W/DSG-PEG2000 (1:3 molar ratio) without the acetate buffer is about 0.1 mg/mL. The solubility of the free base with acetate buffer is >8 mg/mL.

### Example 4: Preparation of Dosing Solutions of AM-2-19-OAc

Dosing solutions were prepared using a micellar solution vehicle of 5% dextrose in water (D5W) containing distearoyl-rac-glycerol PEG 2000 (DSG-PEG 2000). DSG-PEG 2000 is a PEGylated lipid polymeric excipient which forms mixed micelles when formulated with AM-2-19 and functions to solubilize and to stabilize the drug.

### Assumptions:

Dose levels: 0.3, 1.0, 3.0 and 7.0 mg/kg
Dog weight—10 kg
Dose volume—1 mL/kg (IV bolus)
Concentrations—0.3, 1.0, 3.0 and 7.0 mg/mL AM-2-19 free base.
Bolus Infusion—10 mL administered to each animal
Sterile filtration is required for this study

### Drug:

AM-2-19 acetate salt (Molecular weight: 1057.24 g/mol). Note, the molecular weight of AM-2-19 free base is 997.19 g/mol.
Purity and correction factor of AM-2-19 acetate salt is provided with the Certificate of Analysis.

### Vehicle Components:

Dextrose 5% (D5W) USP should be purchased from a commercial source. This is also known as D-glucose 5% (w/w)
Distearoyl-rac-glycerol PEG 2000 (DSG-PEG 2000) (Molecular weight 2621.4 g/mol).
   Note, the mean density of the vehicle over the range of DSG-PEG 2000 concentrations used in this study is 1.026 g/mL.

### Dose Formulation Preparation

Refer to the Tables below for amounts of the drug (AM-2-19 free base) and DSG-PEG 2000 to prepare the vehicle and dosing solutions. Note, the drug is provided as the acetate salt, so a correction factor provided on the CoA must be used to calculate the amount of acetate salt to weigh out. The procedure is written to prepare a stock solution at the highest drug concentration which is then sterile filtered. The lower dosing concentration solutions are prepared by dilution (in D5W/glucose 5% (w/w)) of the high concentration solution.

If desired, the vehicle solution (DSG-PEG 2000 in D5W) can be prepared in advance, 0.22 um filtered, and stored between use in the refrigerator (2-8° C.). The vehicle solution is stable for 7 days.

The dosing solutions must be prepared fresh on each day of dosing. The stability of the dosing solution is such that it should be prepared and dosed within 6 hours.

The following are instructions for the preparation of 100.0 mL of the vehicle formulation. The volume can be scaled as required up to 750 mL (the limit of our experience):

4A. Vehicle Preparation (100 mL of 55.20 mg/mL DSG-PEG 2000)
a. Accurately pipette (or weigh) 100.0 mL of D5W into an appropriate glass container
b. Insert a stir bar into the container and begin stirring the D5W.
c. Accurately weigh 5.52 g of DSG-PEG 2000 and transfer into the D5W. It may help to add the DSG-PEG 2000 in portions to prevent agglomeration/clumping.
d. Sonicate for 5 min at room temperature to facilitate dissolution of the DSG-PEG 2000.
e. Stir the solution on a stir plate at room temperature for at least 15 min to ensure complete dissolution of the DSG-PEG 2000.
f. If necessary, sonicate the vehicle for an additional 5 min and then stir for 15 min or longer, until the DSG-PEG 2000 is completely dissolved. The micellar solution formed should be clear.
g. Measure and record the final pH.
h. If DSG-PEG 2000 solution is being prepared for use on a single day, then skip steps i. and j.
i. Filter the DSG-PEG 2000 solution with a 0.22 um filter. If needed, a larger Millex GV filter (e.g. 33 mm) than specified above of the same Durapore PVDF membrane can be used.
j. When prepared in this way, the vehicle solution can be prepared in advance and stored for 7 days in the refrigerator (2-8° C.).

### 4B. Dosing Solutions Preparation

The dose formulations will be prepared fresh on the day of dosing. First, a stock solution of the highest drug concentration (7.0 mg/mL) to be dosed is prepared and sterile filtered. Lower concentration dosing solutions are prepared by subsequent dilutions using D5W as the diluent. The test item dose formulations will be prepared under a laminar flow hood using clean techniques. These formulations are light sensitive. Therefore, all containers will be protected from light during preparation.

Stock Solution Preparation (60.0 mL of 7.0 mg/mL Dosing Solution)
k. Accurately weigh 420.0 mg of the test item (AM-2-19) into a suitable glass container (Note: use the correction factor provided on the CoA to calculate the amount of AM-2-19 acetate salt to weigh). AM-2-19 acetate salt is fluffy and may require mitigation of static charge.
l. Accurately add 60.0 mL of the vehicle solution (at room temp.) to dissolve the drug.
m. Place the container in a water bath placed on a heating stir plate, cover the container and keep it covered in order to minimize evaporation and start stirring.
n. Heat the water until it reaches a temperature of 50±2° C. while constantly monitoring the temperature by placing a thermometer in the water bath.
o. Once the water bath reaches a temperature of 50±2° C., continue stirring the formulation for an additional 30 min. Monitor the temperature and use ice or cold water, if necessary, to maintain the temperature of the water bath at 50±2° C.
p. Remove the flask containing the dose formulation from the water bath and allow the solution to cool down to room temperature by placing the container in an ice bath or by placing it in a fume hood, while occasionally swirling the flask.
q. Filter the dose formulation solution with a 0.22 um PVDF syringe filter (33 mm membrane size) after it is equilibrated to room temperature (15 to 30° C.), discarding the first 2 mL. Lower solution concentrations do not require filtration and should be prepared under a laminar flow hood using clean techniques.

Recommended dilutions to prepare 35.0 mL of lower concentration dosing solutions are shown in the Table below. For example, to prepare the 3.0 mg/mL dosing solution, the 7.0 mg/mL stock solution made in steps 2.a-2.g above, is diluted 1:2.33 using D5W (15.0 mL stock+20.0 mL D5W).

| To prepare 4 | Use this | | | |
|---|---|---|---|---|
| mL of this AM- | solution | | | |
| 2-19 | concentration | | Aliquot | |
| concentration | for dilution | Dilution | Volume | Volume |
| (mg/mL) | (mg/mL) | factor | (ml) | D5W |
| 3.0 | 7.0 | 1:2.33 | 15.0 | 20.0 mL |
| 1.0 | 7.0 | 1:7 | 5.0 | 30.0 mL |
| 0.3 | 7.0 | 1:23 | 1.50 | 33.5 mL |

The elapsed time between preparation of the dosing solutions and administration should be less than 6 hours.

### Example 5: Characterization of the AM-2-19-OAc-DSG-PEG-2000 Formulation

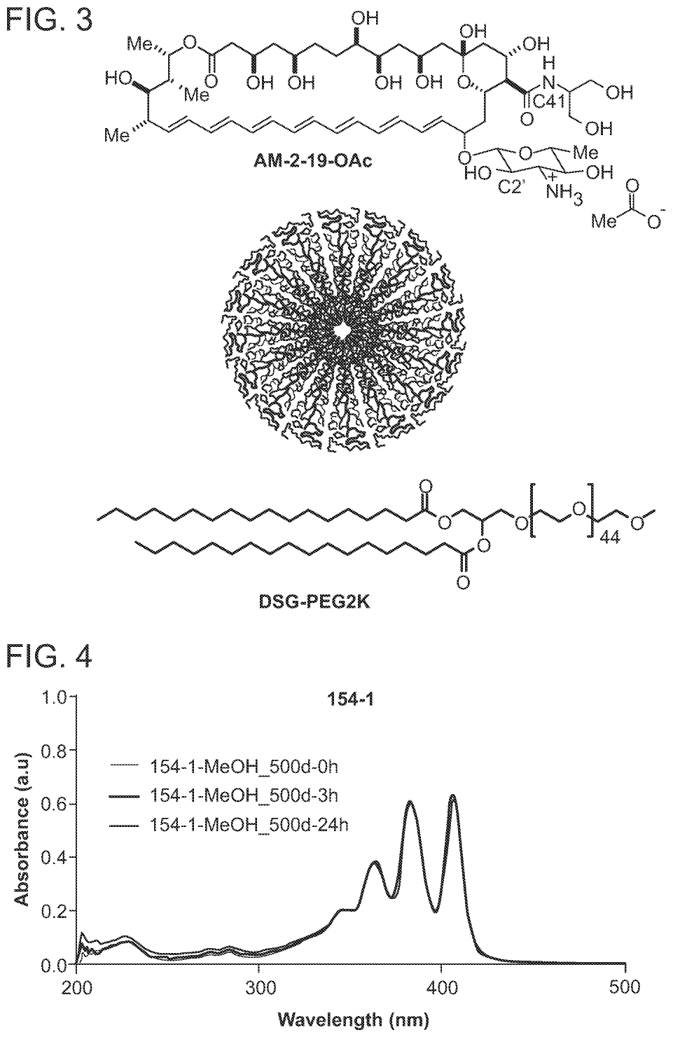

Structures of AM-2-19-OAc and DSG-PEG-2000 are shown in FIG. 3.

Micellar size was characterized by dynamic light scattering (DLS), which demonstrated stability of the micelles over time, as shown below.

| DLS (0 h; d nm)* | DLS (3 h; d nm)* |
|---|---|
| 6.48 (100%) | 7.54 (100%) |

*Based on volume distribution

As shown in FIG. 4, there is no marked change in the UV spectrum over time, indicating stability of the DSG-PEG 2000 micellar formulations of the AM-2-19-OAc. Indeed, after 24 h, there was a retention >98% of the initial concentration.

### Example 6: Aqueous Solution Stability of AM-2-19-OAc in Comparison with AM-2-19-OAc-DSG-PEG-2000

The aqueous solution stability of AM-2-19-OAc and AM-2-19-OAc-DSG-PEG-2000 stock solutions in D5W was evaluated at 2.5 mg/mL. The AM-2-19-OAc-DSG-PEG-2000 stock solution was prepared by adding AM-2-19-OAc to a solution of DSG-PEG 2000 in D5W to prepare a 1:3 (drug:excipient) composition, which was then stirred at 50° C. for 30 min to form a micellar formulation.

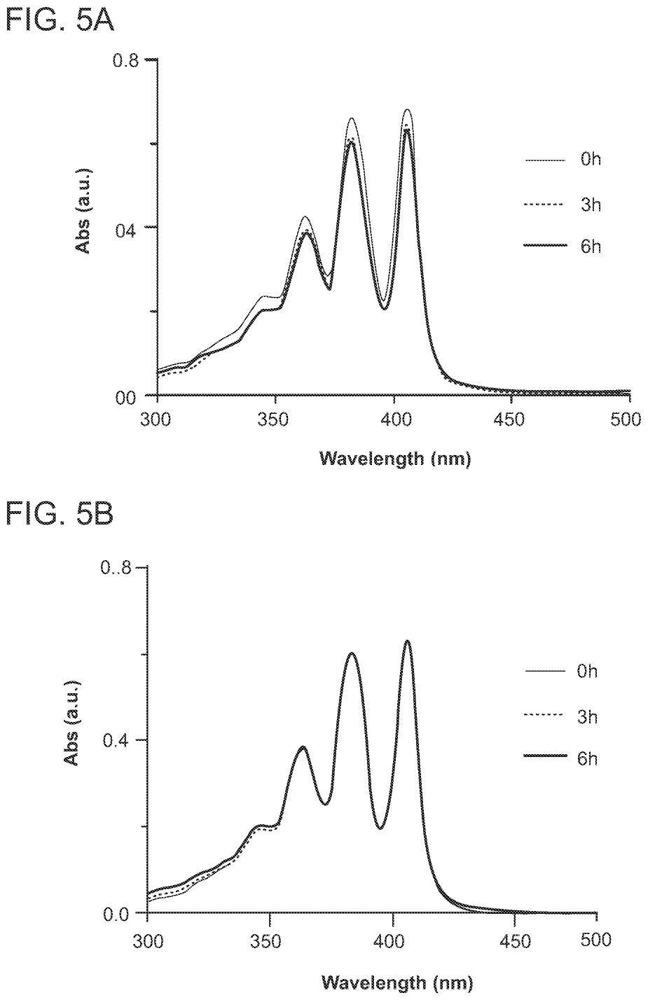

To measure the solution stability of each solution, aliquots were carefully collected from the top of each stock solution at predetermined time points and diluted 500-fold with methanol for UV measurements. In the AM-2-19-OAc stock solution, >10% loss of concentration was recorded over 6 h (FIG. 5A). In contrast, the AM-2-19-OAc-DSG-PEG-2000 stock solution remained stable and no significant loss of concentration was observed after 6 h at room temperature (FIG. 5B).

### Example 7. Solution Behavior of AM-2-19-OAc-DSG-PEG-2000

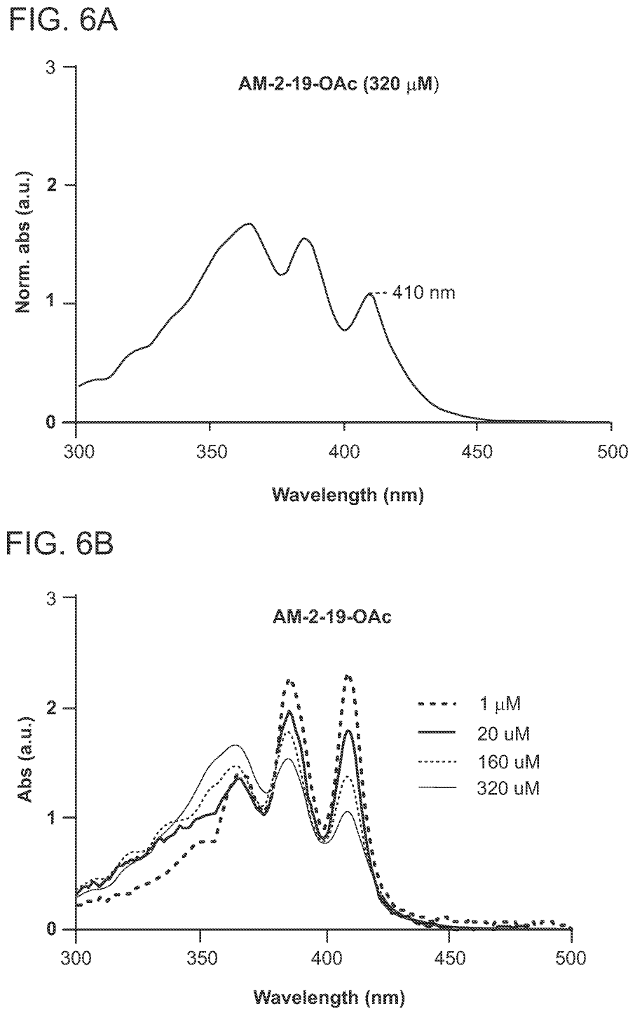

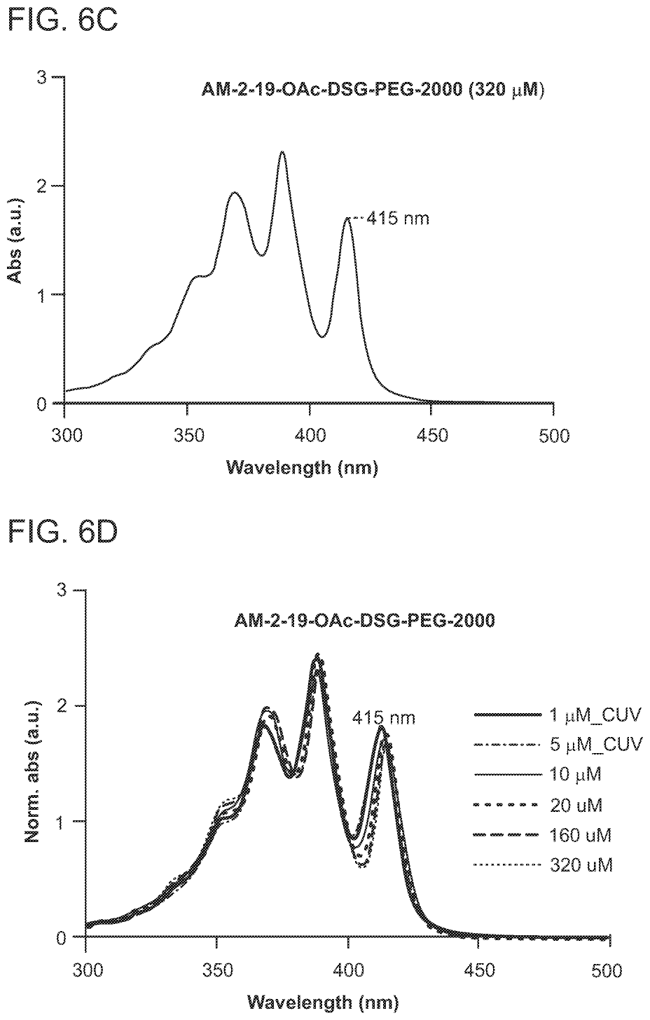

The solution behavior of AM-2-19-OAc-DSG-PEG-2000 was evaluated using UV-vis and NMR techniques. The UV pattern of AM-2-19-OAc solution was found to be concentration dependent where monomer-like UV at lower concentration shifted to an aggregate-like pattern at higher concentration with an absorption peak at 410 nm (FIGS. 6A & 6B). In AM-2-19-OAc-DSG-PEG-2000, no such concentration-dependent change in UV was observed and a sharp absorption peak at 415 nm appeared which was hypothesized to originate from the drug, encapsulated in the micelles (FIGS. 6C & 6D).

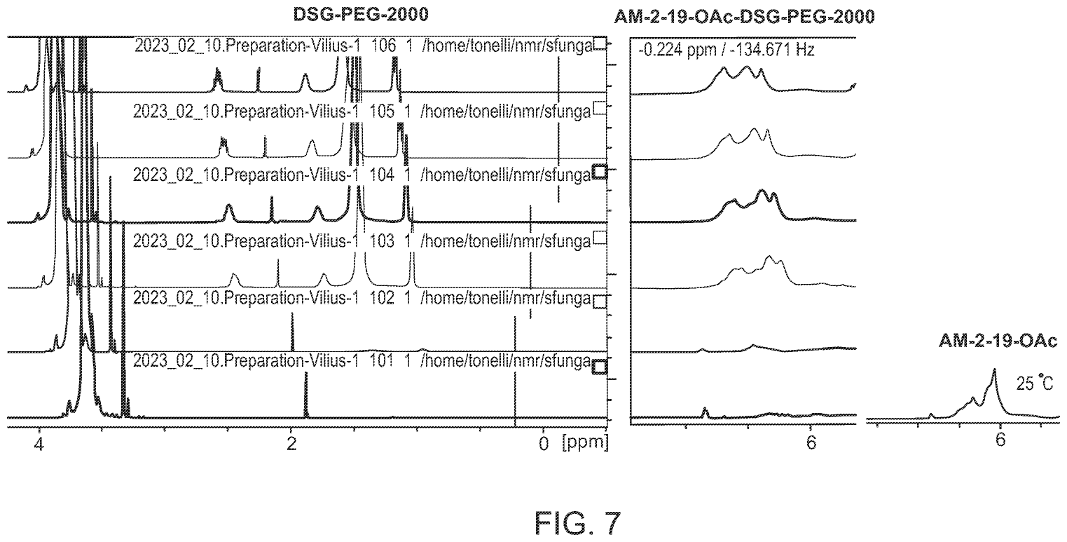

To further understand the encapsulation phenomenon, solutions of AM-2-19-OAc, AM-2-19-OAc-DSG-PEG-2000, and DSG-PEG-2000 were prepared in deuterated D5W and probed by ¹H-NMR under variable temperatures. In the DSG-PEG-2000 control, no characteristic peak was observed up to 35° C. However, at ˜40° C., distinct peaks appeared, likely due to micellar phase transition (FIG. 7). See Otten, D. et al. Biophysical Journal, 1995, 68 (2), 584-597. Upon conducting the same experiment with AM-2-19-OAc-DSG-PEG-2000, no characteristic AM-2-19 peaks were observed between 25-36° C. (FIG. 7). However, at ˜40° C., which corresponds to the micellar phase transition, polyene signals of AM-2-19 started to emerge consistent with the faster relaxation of the encapsulated drug molecules during the phase transition of DSG-PEG-2000 (FIG. 7). In the absence of DSG-PEG 2000 micelles, the polyene signals of AM-2-19 can be seen at 25° C. (FIG. 7). These results suggest that most of the drug molecules are encapsulated in AM-2-19-OAc-DSG-PEG-2000.

### Example 8:˜Broad Spectrum Antifungal Activity of the AM-2-19-OAc-DSG-PEG-2000 Formulation

The AM-2-19-OAc-DSG-PEG-2000 formulation was tested against various fungal strains and compared to AmB, AM-2-19-OAc (in vehicle), and DSG-PEG-2000 micelles (no API). Results are given in the table below:

| | | AM-2-19- | DSG-PEG 2k + | DSG-PEG 2k |
|---|---|---|---|---|
| | AmB | OAc | AM-2-19-OAc | control |
| C. albicans SN250 | 0.375 | 1 | 0.25 | >16 |
| C. albicans | 0.25 | 1 | 0.25 | >16 |
| C. glabrata | 0.125 | 0.5 | 0.094 | >16 |
| C. krusei | 0.5 | 1 | 0.5 | >16 |
| C. tropicalis | 0.5 | 0.5 | 0.125 | >16 |
| A. fumigatus 91 | 2 | 2 | 1 | >16 |
| A. fumigatus 1100 | 2 | 2 | 1 | >16 |
| A. fumigatus 1163 | 1.5 | 2 | 1 | >16 |

Surprisingly, DSG-PEG-2000 increases the potency of AM-2-19-OAc in vitro.

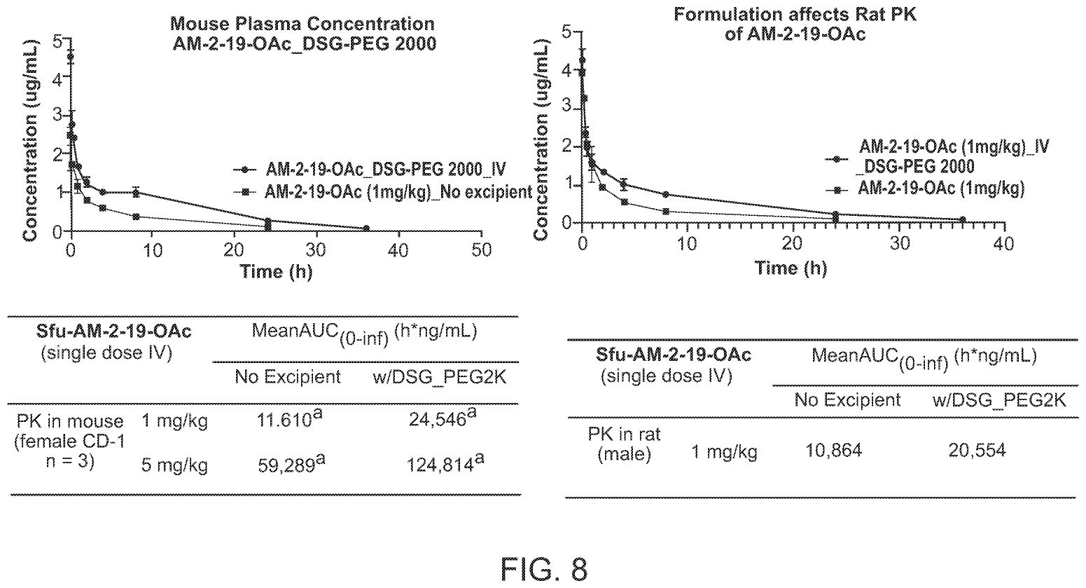

Additionally, DSG-PEG-2000 increases the half-life of AM-2-19-OAc in vivo as compared to the half-life of AM-2-19-OAc when not in the micellar formulation (see FIG. 8).

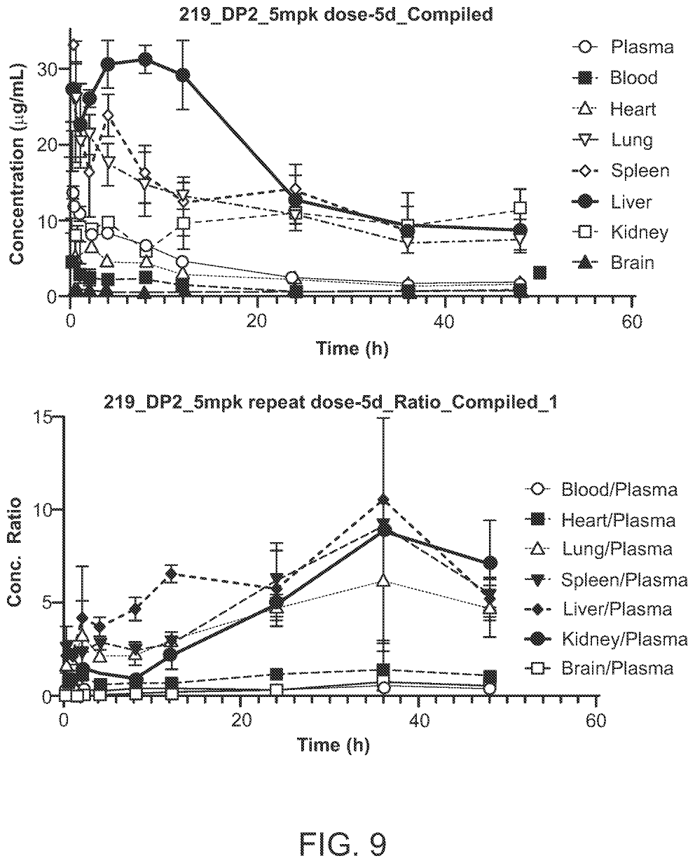

The micellar formulation also provided favorable tissue distribution data in mice (see FIG. 9).

### Example 9: AM-2-19-OAc-DSG-PEG-2000 Retains Characteristic UV Pattern in RPMI

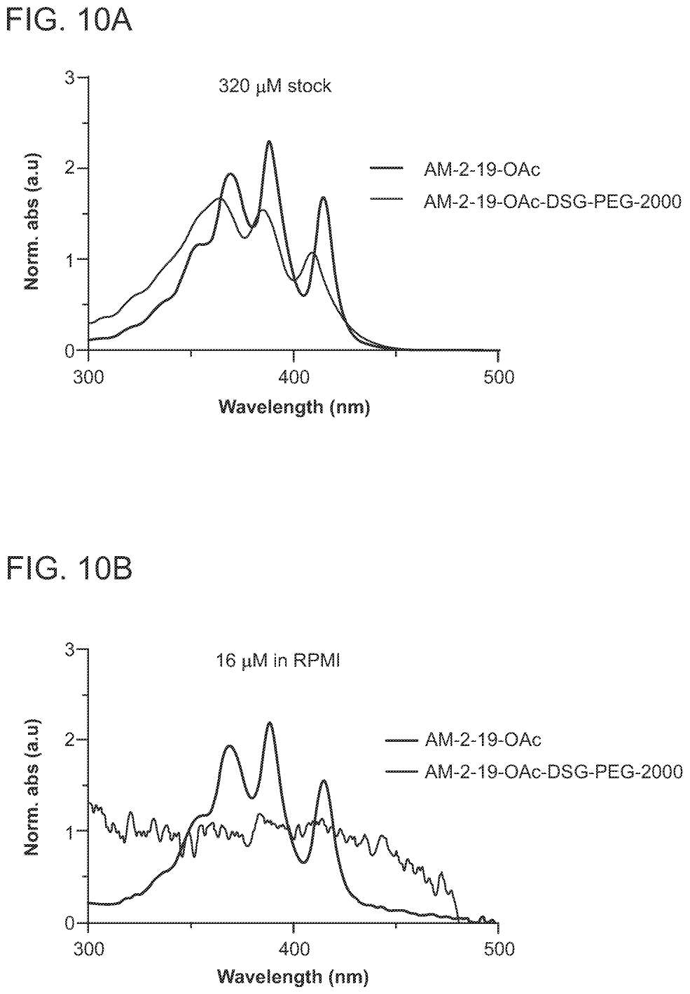

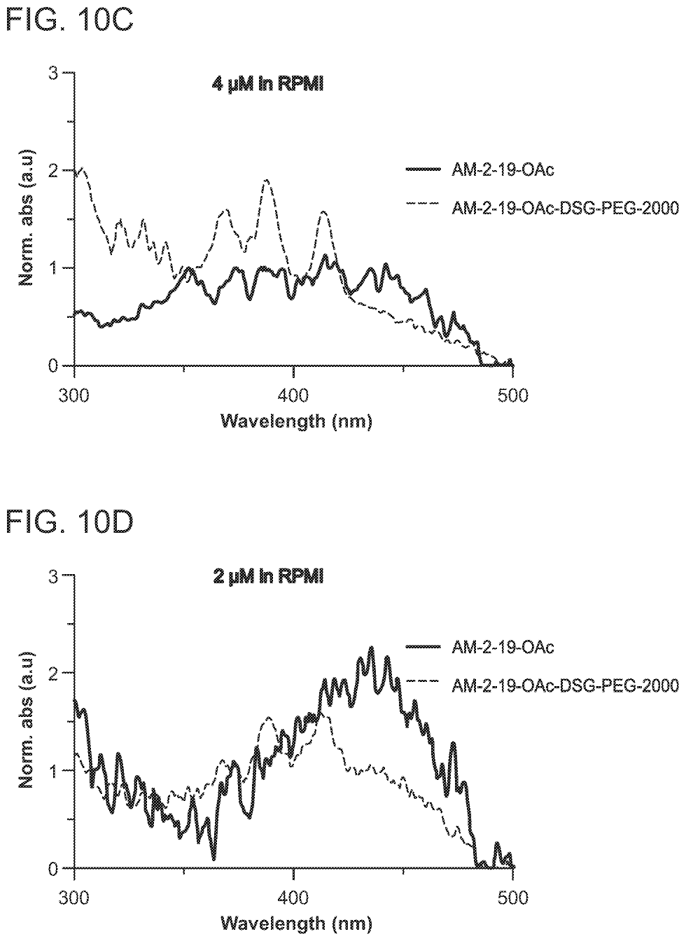

To probe the structure of AM-2-19-OAc-DSG-PEG-2000 under biologically relevant conditions, 320 μM stock solutions of AM-2-19-OAc and AM-2-19-OAc-DSG-PEG-2000 were diluted with RPMI 1640 (pH=7), mimicking the in vitro antifungal efficacy evaluation condition. The characteristic 415 nm UV peak of micellar AM-2-19-OAc-DSG-PEG-2000 was retained at >2 M concentration, whereas no characteristic AM-2-19-OAc 410 nm UV peak was observed in the diluted samples (FIGS. 10A-10D). The retention of micellar structure at these concentrations is consistent with the improved MIC of AM-2-19-OAc-DSG-PEG-2000 as compared with AM-2-19-OAc against fungal isolates.

### Example 10: The AM-2-19 in AM-2-19-OAc-DSG-PEG-2000 Binds to Plasma Proteins

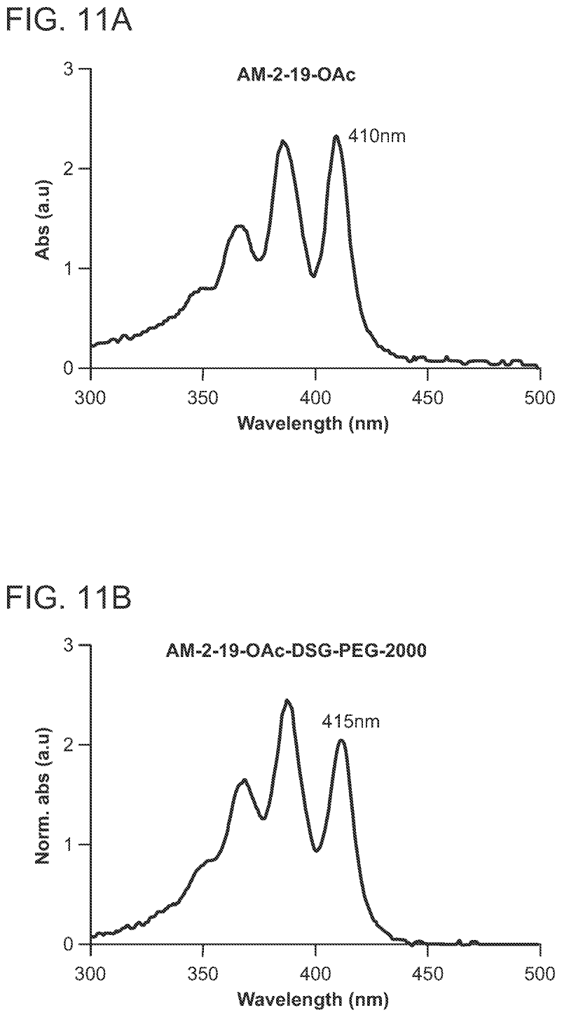

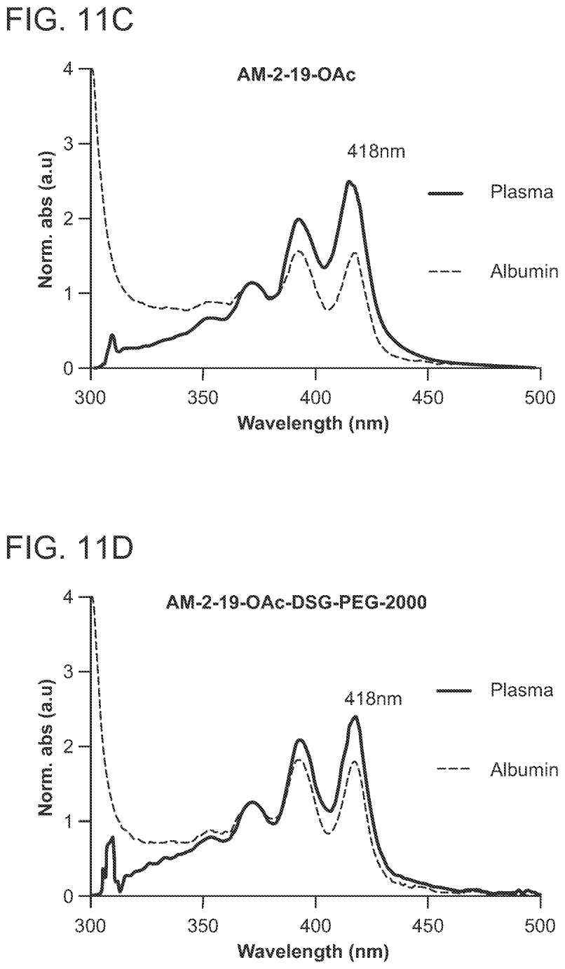

The impact of plasma dilution and albumin titration on AM-2-19-OAc-DSG-PEG-2000 was investigated, which mimics the fate of drug molecules post-IV dosings or infusion. The UV patterns of stock solutions of AM-2-19-OAc and AM-2-19-OAc-DSG-PEG-2000 included their respective characteristic peaks at 410 nm and 415 nm (FIGS. 11A & 11B). Upon dilution with plasma, however, the UV pattern of both AM-2-19-OAc and AM-2-19-OAc-DSG-PEG-2000 solutions provided a characteristic 418 nm peak, which is indicative of AM-2-19 binding to the plasma proteins (FIGS. 11C & 11D).

A similar peak was also observed when both of these solutions were titrated using Human serum albumin (FIGS. 11C & 11D). See Xie, M. et al. Biochim Biophys Acta. 2006, 1760(8), 1184-91.

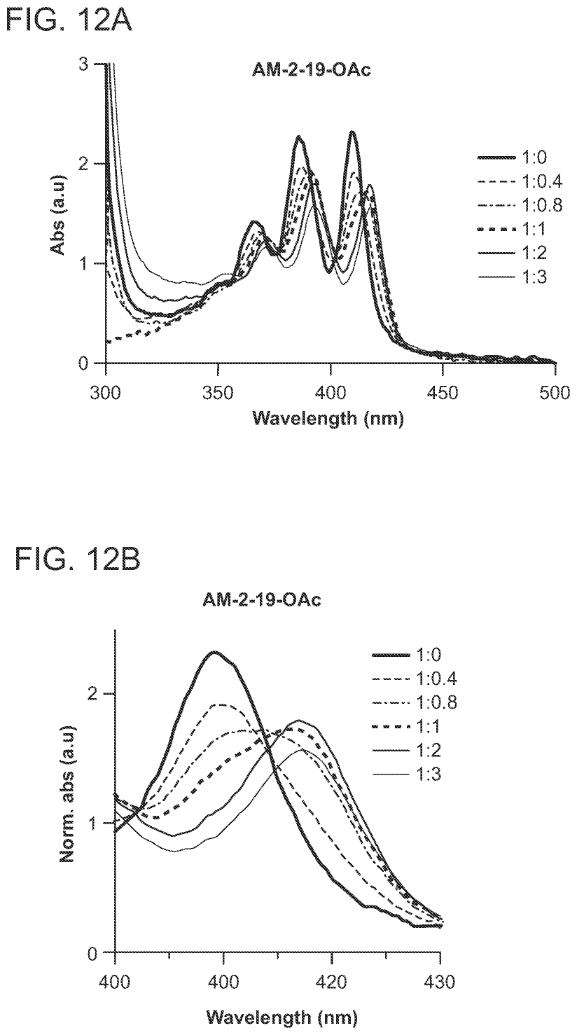

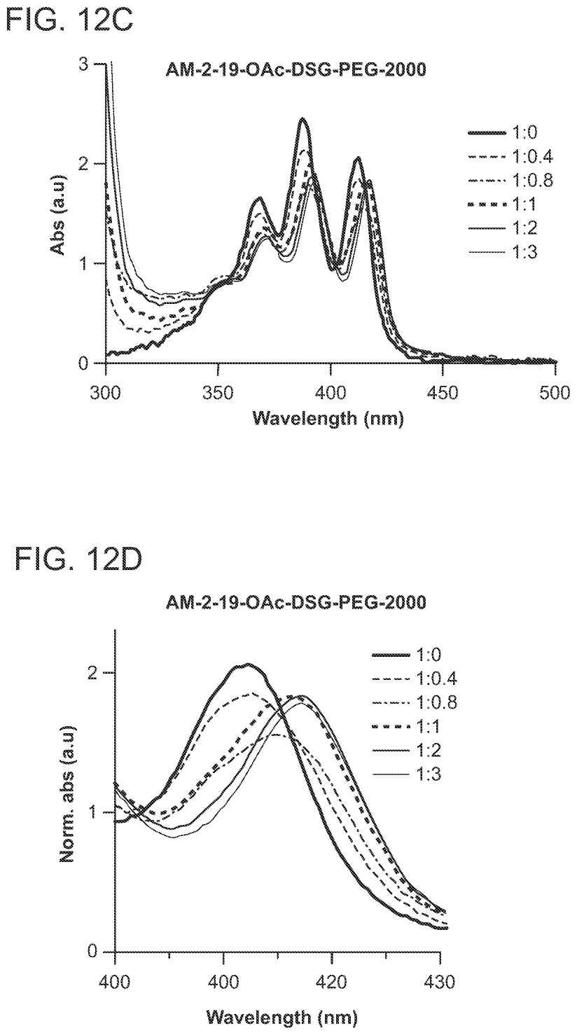

Both solutions were titrated with Albumin and the UV spectrum of the resulting solutions were measured at various ratios of AM-2-19 to Albumin (FIGS. 12A-12D). A shift in the UV pattern for each solution starts to appear at a 1:0.8 ratio. For the titration of AM-2-19-OAc, a broad transition of 410 nm to 418 nm peak was recorded at 1:1 ratio (FIGS. 12A & 12B), whereas AM-2-19-OAc-DSG-PEG-2000 yields a sharp bathochromic shift of 415 nm to 418 nm at 1:1 ratio (FIGS. 12C & 12D), which suggests that AM-2-19-OAc-DSG-PEG-2000 efficiently hands off AM-2-19 to albumin.

These data indicate that AM-2-19-OAc-DSG-PEG-2000 offers improved aqueous stability and plasma compatibility and retained micellar structure in biologically relevant concentration without disrupting binding to plasma proteins.

### Example 11: Analysis of Toxicity Biomarkers

A panel of formulations of AM-2-19-OAc, other AmB formulations, and control formulations were assessed for toxicity.

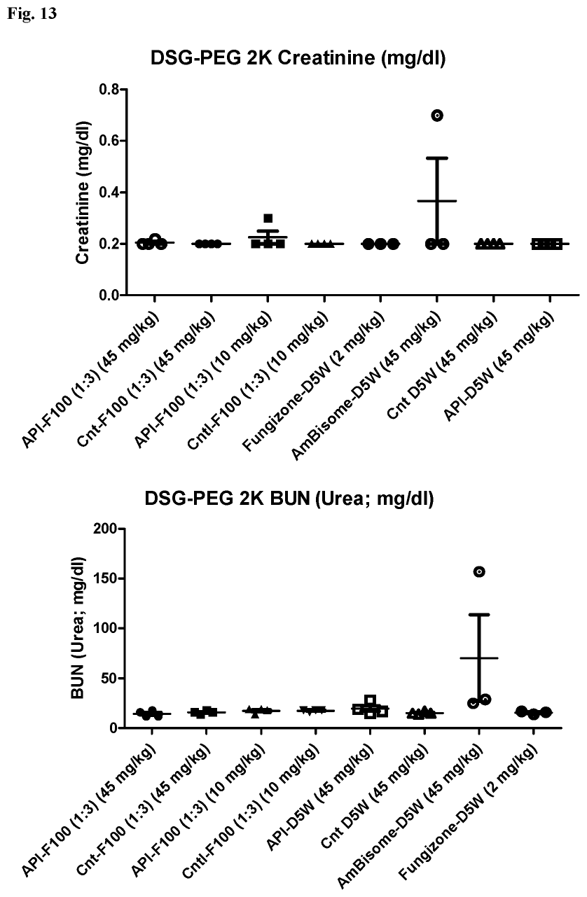

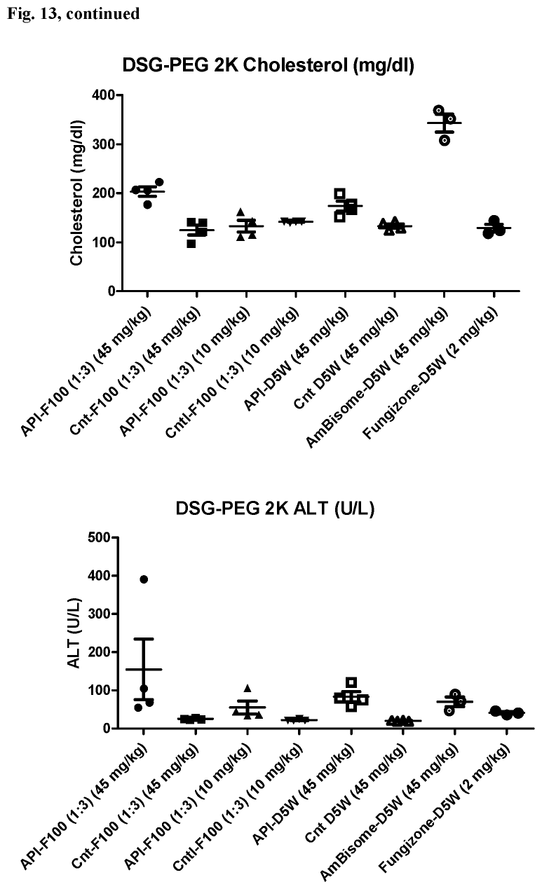

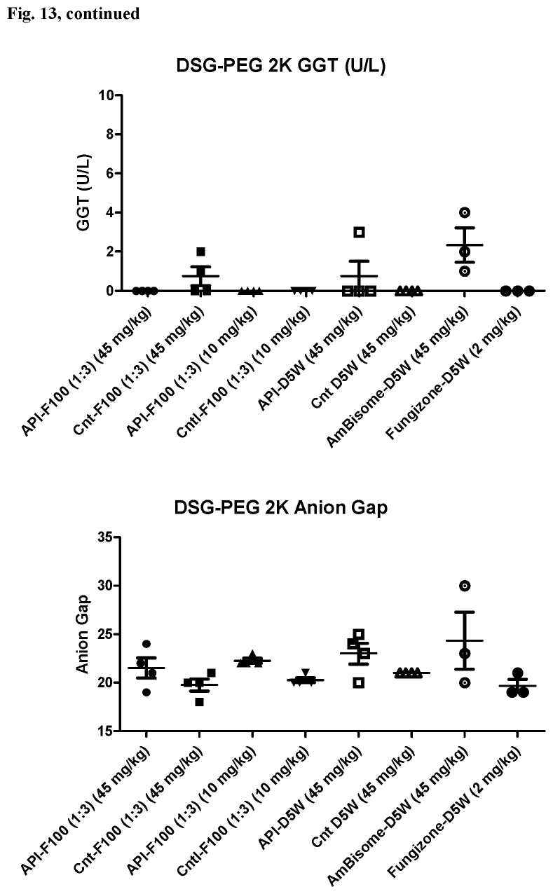

As shown in FIG. 13, AM-2-19-OAc-DSG-PEG-2000 does not increase common toxicity biomarkers.

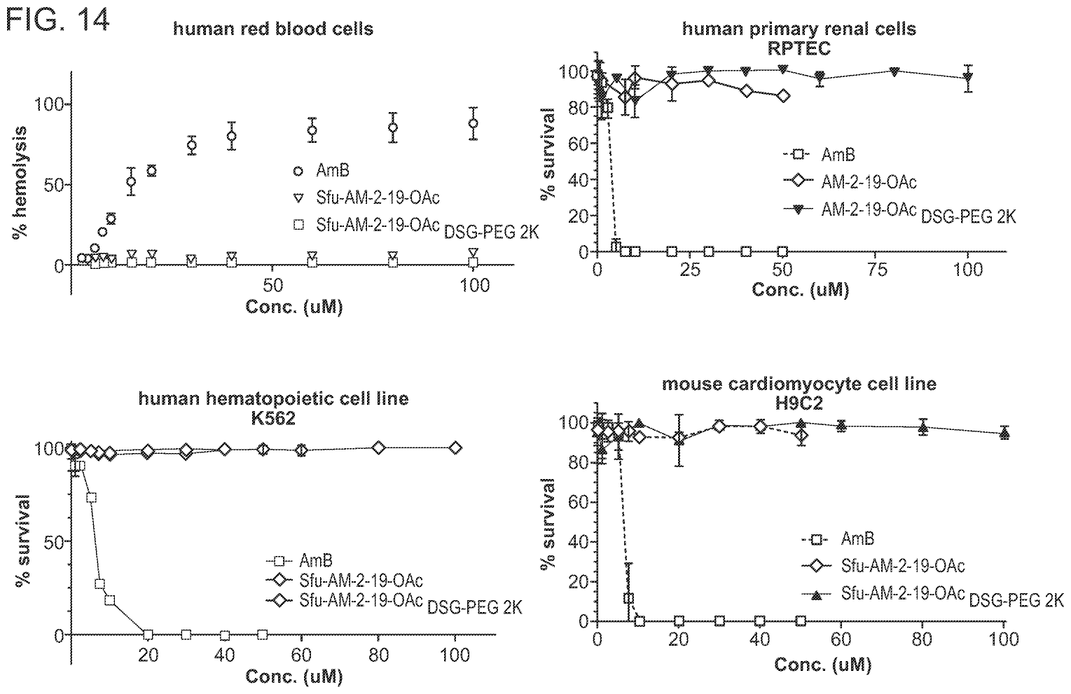

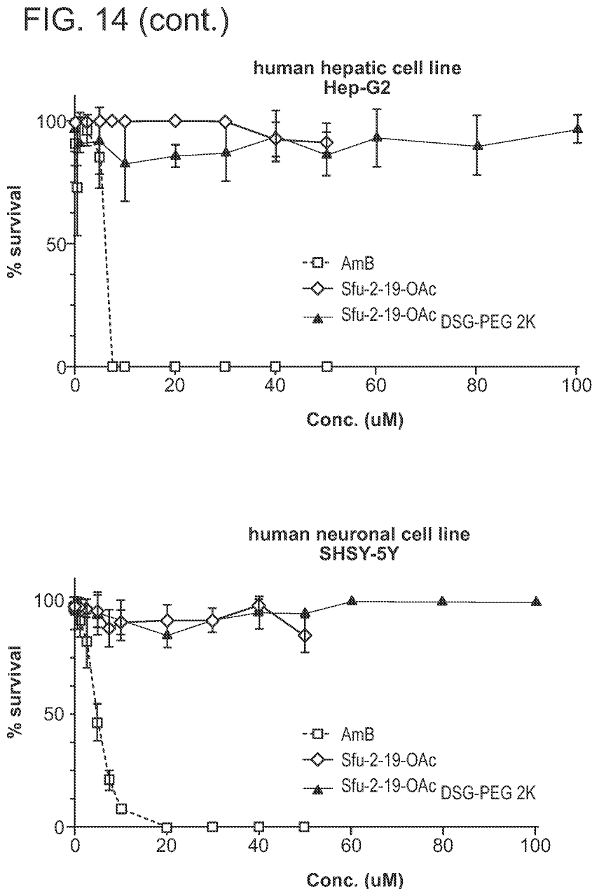

FIG. 14 shows that AM-2-19-OAc-DSG-PEG-2000 retains loss of toxicity in vitro.

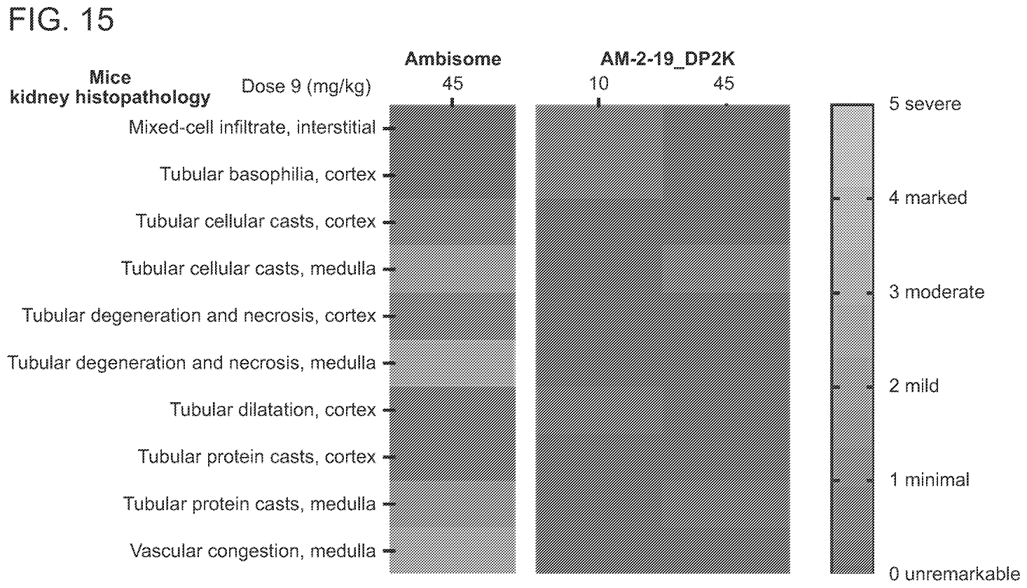

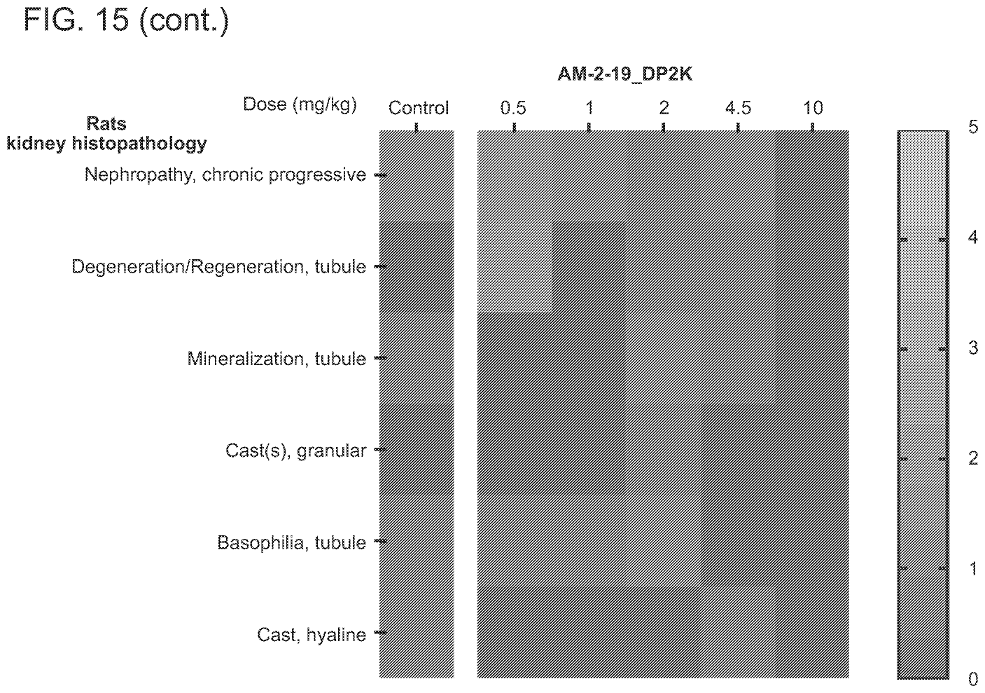

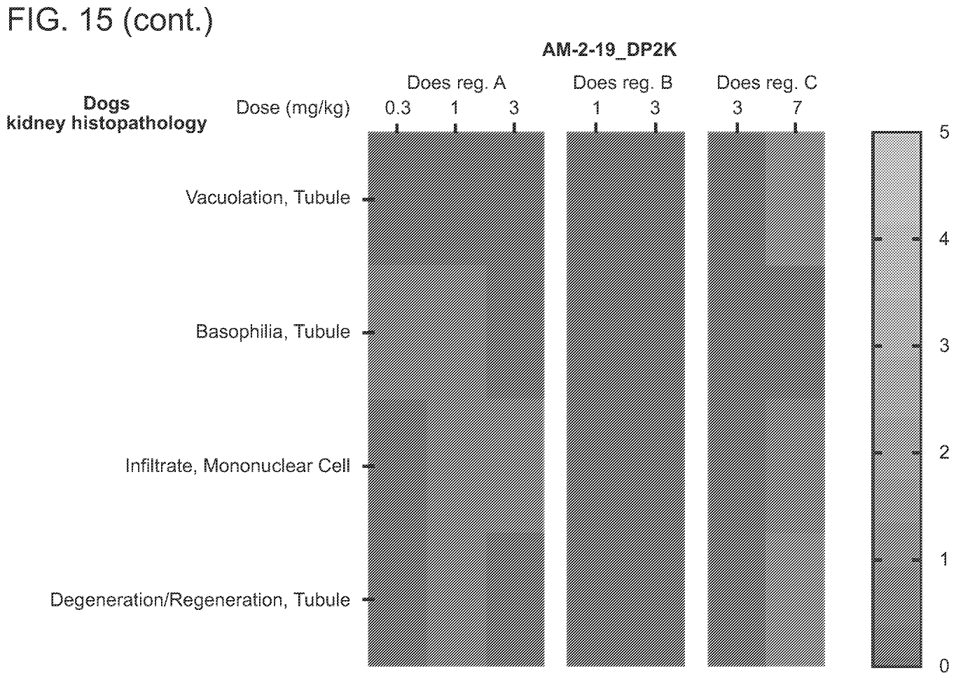

FIG. 15 shows a collection of histopathology charts demonstrating that the AM-2-19-OAc-DSG-PEG-2000 formulation does not cause kidney damage in mice, rats, or dogs.

### Example 12: Toxicokinetics

The toxicokinetics and tolerability of a single dose of AM-2-19-OAc-DSG-PEG-2000 in rats was studied. The following table summarizes the experimental setup.

| | | | | | | Number of | | | |
|---|---|---|---|---|---|---|---|---|---|
| | | | | | | Rats/Sex | | | |
| Group - | Method of | Dose | Dose Conc | Dose Vol. | Infusion rate | Main^{a} | | TK | |
| Treatment | Admin. | (mg/kg) | (mg/mL) | (mL/kg) | (mL/kg/h) | M | F | M | F |
| 1: Vehicle | 1 hour | 0 | 0 | 10 | 10 | 6 | 6 | 3 | 3 |
| 2: AM-2-19^{a} | continuous | 0.2 | 0.02 | 10 | 10 | — | 6 | — | 9 |
| 3: AM-2-19 | IV infusion | 0.5 | 0.05 | 10 | 10 | 6 | 6 | 9 | 9 |
| 4: AM-2-19 | | 1 | 0.1 | 10 | 10 | 6 | 6 | 9 | 9 |
| 5: AM-2-19 | | 2 | 0.2 | 10 | 10 | 6 | 6 | 9 | 9 |
| 6: AM-2-19^{b} | | 4.5 | 0.45 | 10 | 10 | 6 | 6 | 9 | 9 |
| 7: AM-2-19^{c} | | 10 | 1 | 10 | 10 | 6 | — | 9 | — |

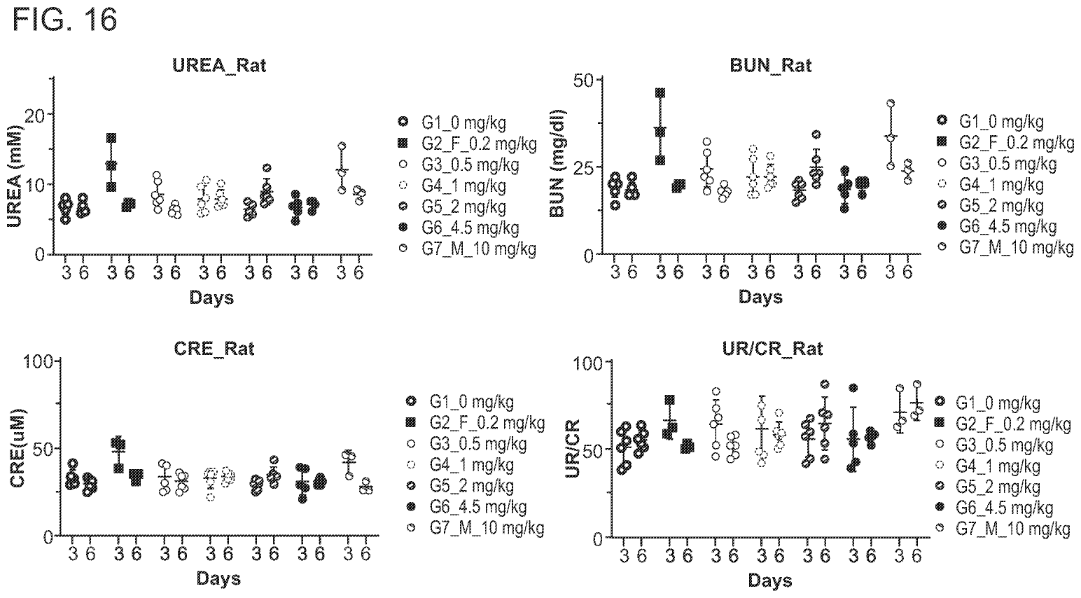

Toxicity biomarkers are shown in FIG. 16

AM-2-19-OAc-DSG-PEG-2000 was also assessed in dogs over a two week period and several dosing schedules. In dosing protocol, all dogs survived all doses with no clinical observations or signs of distress in any dog. There was also no change in food consumption in any dog.

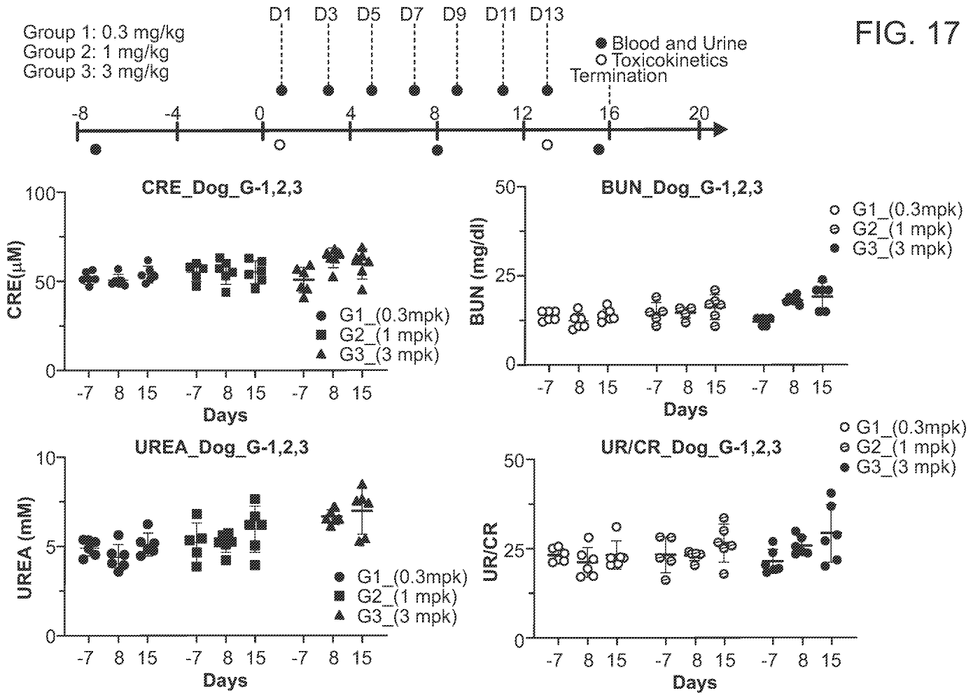

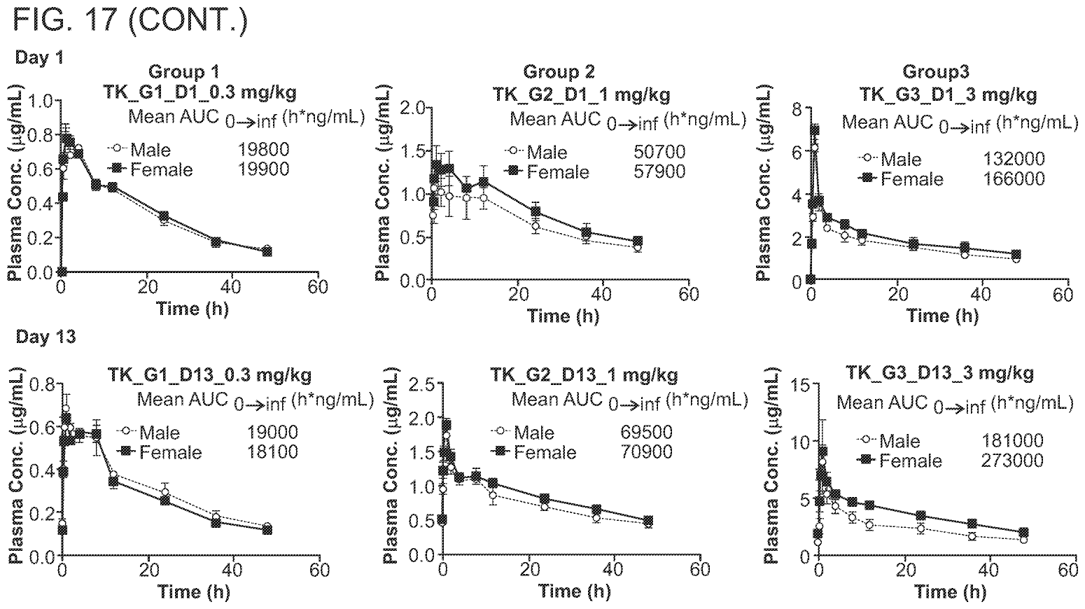

Results of Dosing Regimen A (dose every other day) are shown in FIG. 17.

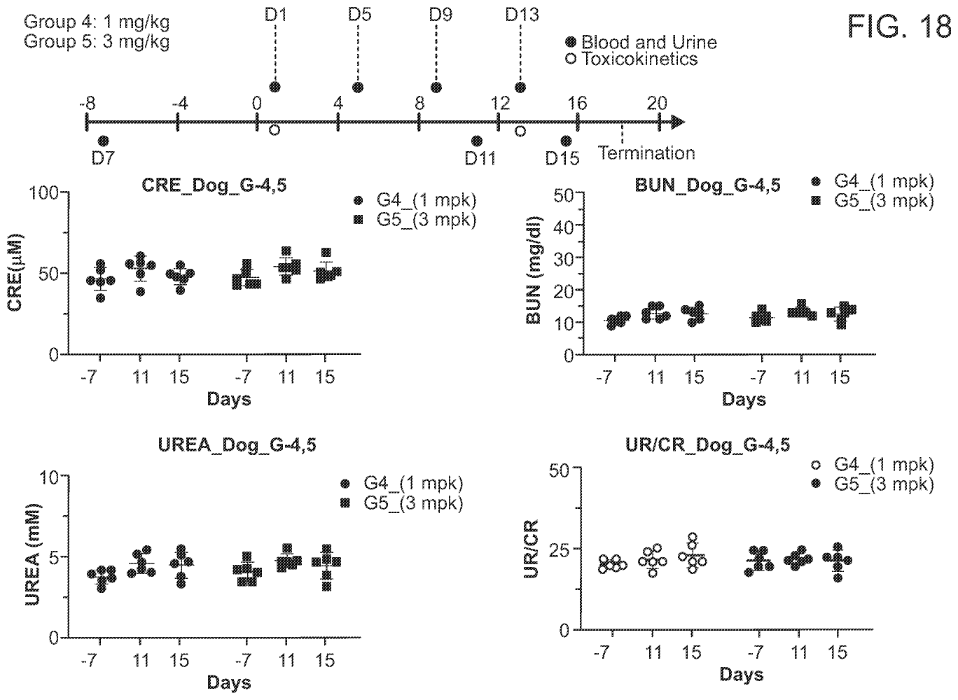

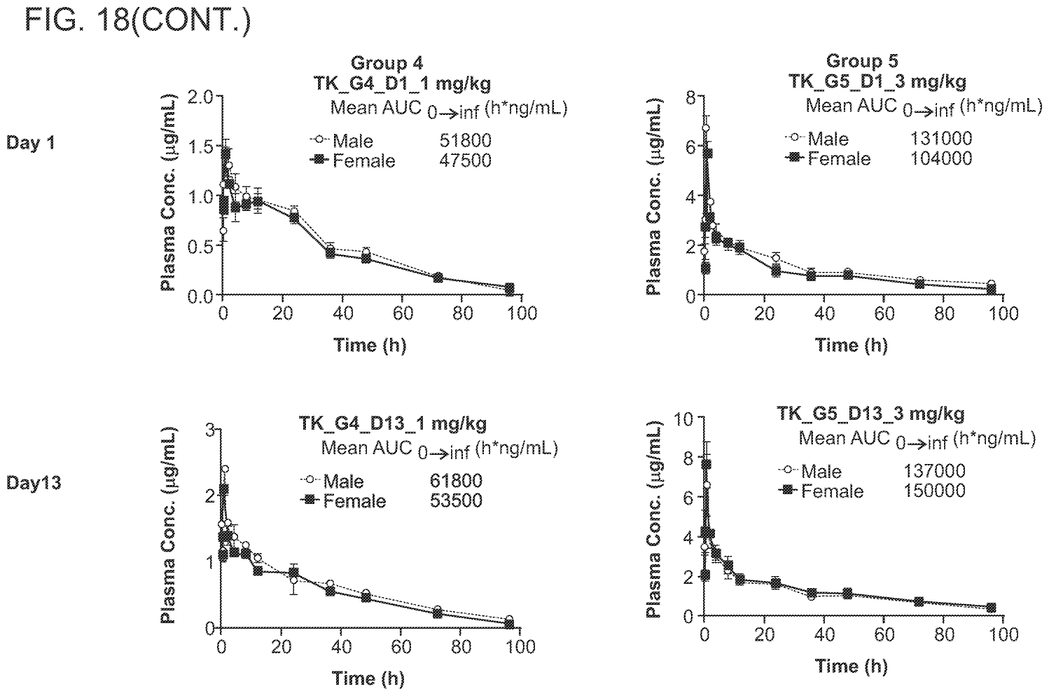

Results of Dosing Regimen B (dose every fourth day) are shown in FIG. 18.

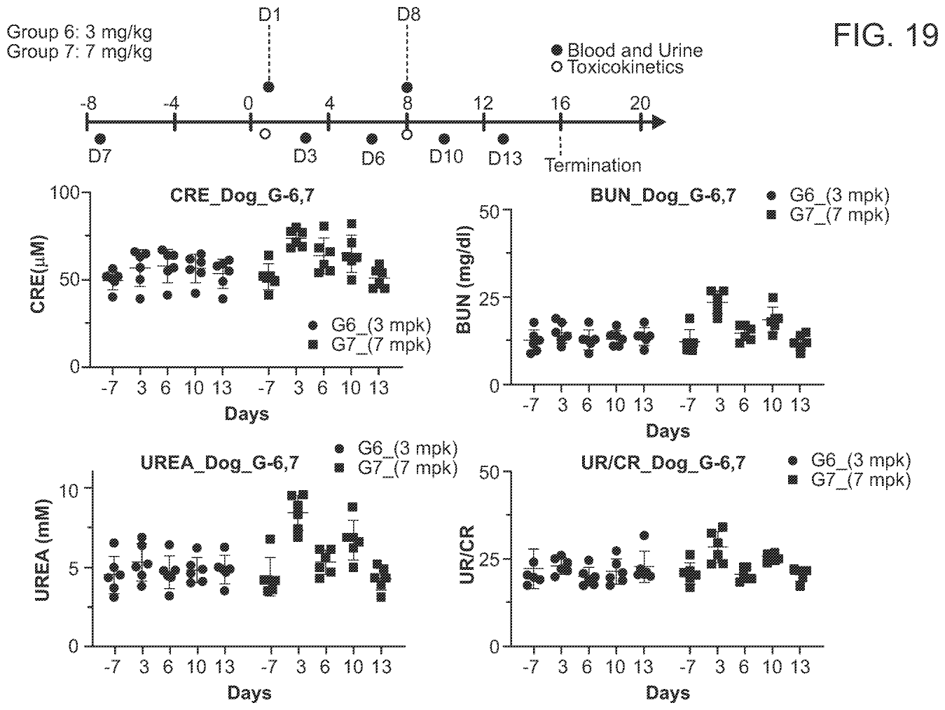

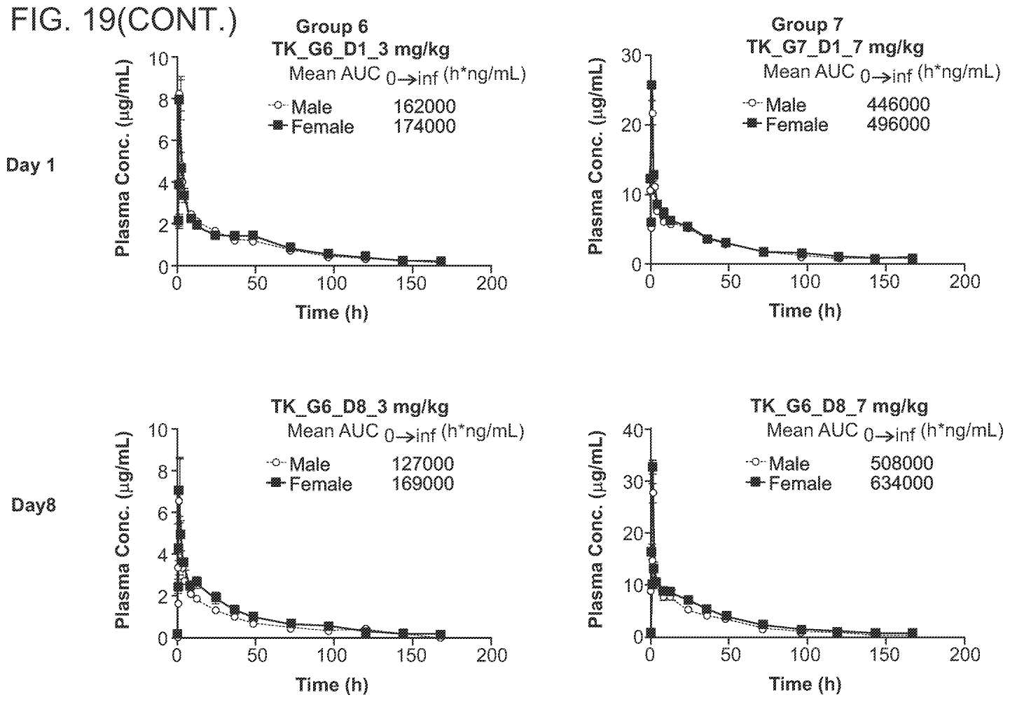

Results of Dosing Regimen C (dose every week) are shown in FIG. 19.

### Example 13: Efficacy of Micellar Formulation

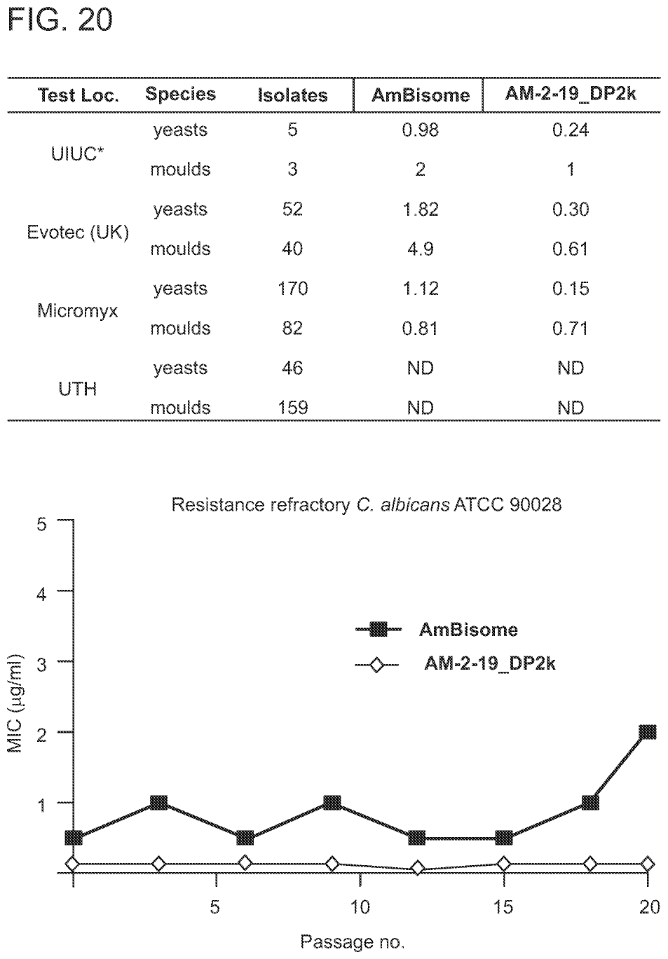

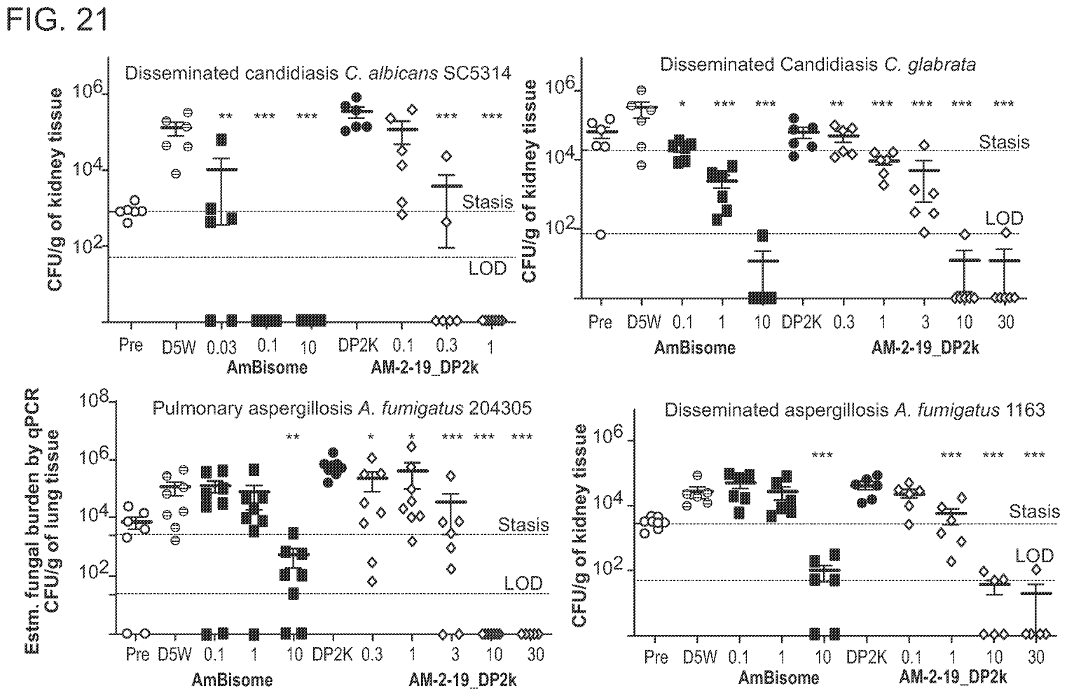

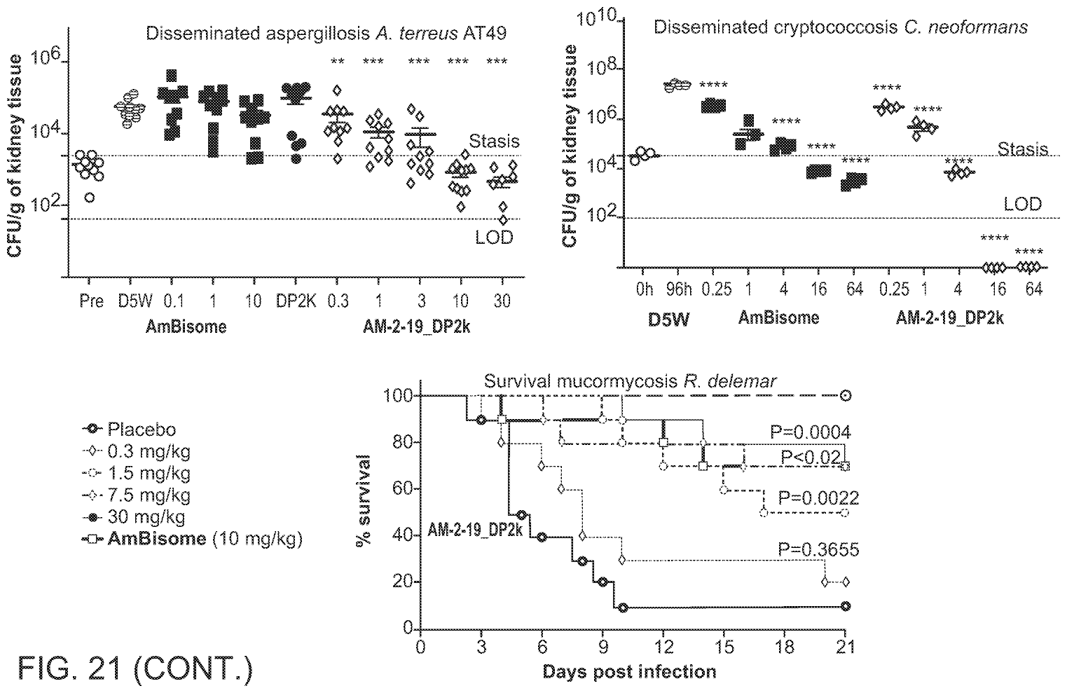

AM-2-19-OAc-DSG-PEG-2000 is a hyperpotent antifungal against a suite of fungal infections including resistance refractory strains and otherwise difficult to treat fungi such as A. terreus. As shown in FIG. 20, AM-2-19-OAc-DSG-PEG-2000 exhibits a lower minimum inhibitory concentration (MIC) than the Amphotericin B liposome formulation AmBisome against numerous strains of yeasts and moulds, including resistance refractory C. albicans ATCC 90028. As shown in FIG. 21, AM-2-19-OAc-DSG-PEG-2000 dramatically decreases the fungal burden in kidney and lung tissue as compared to control and AmBisome in various fungi strains. In FIG. 21, “Pre” represents the fungal burden at t (time)=0.

### Example 14: Application of Micellar Formulation Platform to Further AmB-Derivatives

The surprising benefits of the DSG-PEG 2000 micellar formulation are not limited to AM-2-19-OAc. Other derivatives of Amphotericin B, including the AmB amides and C2′epi amides having the structures depicted below, were formulated with DSG-PEG 2000.

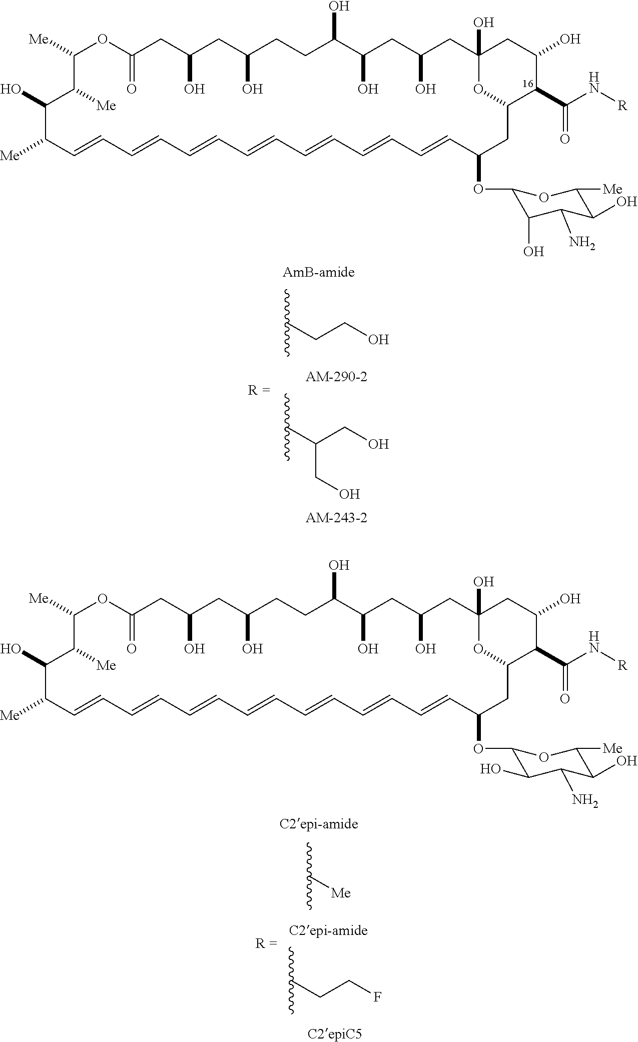

To survey the compounds, 2 mg of the compound were weighed in a clean 7 mL glass vial. Then, 3 equiv. of DSG-PEG 2000 (DP2K) were weighed in a separate 7 mL glass vial. 1 mL D5W was added to the second vial and sonicated for 15 mins to prepare clear solution of DP2K and for the C2′epi compounds filtered through 0.22 m syringe filter. The solution in the first vial with compound (as acetate salt) was transferred to the DP2K solution, and stirred for 30 mins at 50° C. to make a clear yellow solution. The solution was then cooled to room temperature.

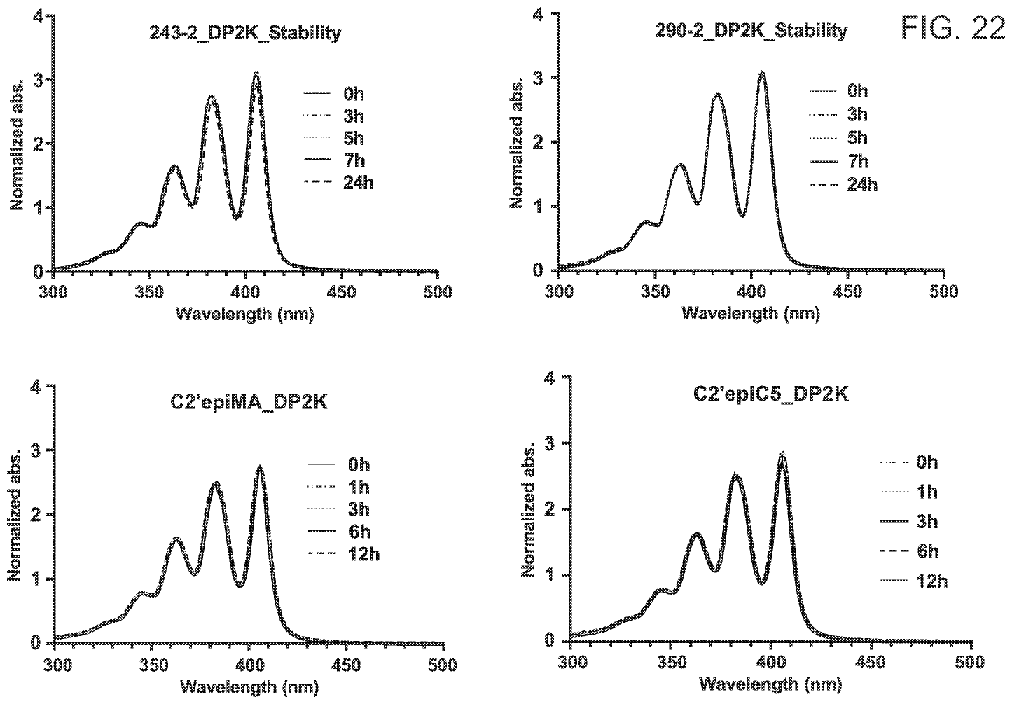

As shown in FIG. 22 and the tables below, the DSG-PEG 2000 micellar formulation also significantly improves the solution stability of AM-290-2, AM-243-2, C2′epiMA, and C2′epiC5. FIG. 22 shows the UV spectrum of the samples having a target concentration of 2 mg/mL. The samples at different time points were prepared by diluting 10 μl aliquot with 990 μl of MeOH (100 fold dilution). As shown, there is no marked change in the UV spectrum over time, indicating stability of the DSG-PEG 2000 micellar formulations of the AmB derivatives.

TABLES
Concentration of AmB derivatives over time in DSG-
PEG 2000 micellar formulation and in D5W vehicle.

| | | Rel. conc. w.r.t time = 0 h | |
|---|---|---|---|
| Samples | Excipient | 7 h | 24 h |
| AM-243-2-Oac_DP2K | DSG-PEG 2K | 99% | 97% |
| AM-243-2-Oac_D5W | — | 91% | 84% |
| AM-290-2-Oac_DP2K | DSG-PEG 2K | 99% | 98% |
| AM-290-2-Oac_D5W | — | 92% | 86% |

| | | Rel. conc. w.r.t time = 0 h | |
|---|---|---|---|
| Samples | Excipient | 6 h | 12 h |
| C2′epiMA-Oac_DP2K | DSG-PEG 2K | 99% | 97% |
| C2′epiMA-Oac_D5W | — | 93% | 85% |
| C2′epiC5-Oac_DP2K | DSG-PEG 2K | 99% | 98% |
| C2′epiC5-Oac_D5W | — | 92% | 80% |

### INCORPORATION BY REFERENCE

All patents and published patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

We claim:

1. A composition, comprising:
(i) a lipid polymer excipient having the structure of formula (X); 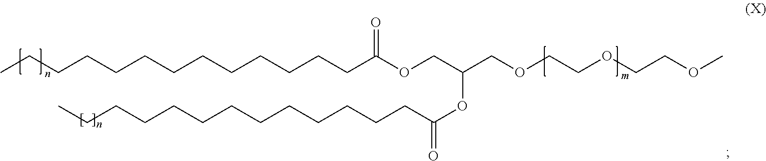
wherein each occurrence of n is independently selected from 0-10; and
m is selected from 10-60; and
(ii) a compound, or a pharmaceutically acceptable salt thereof, selected from the group consisting of: 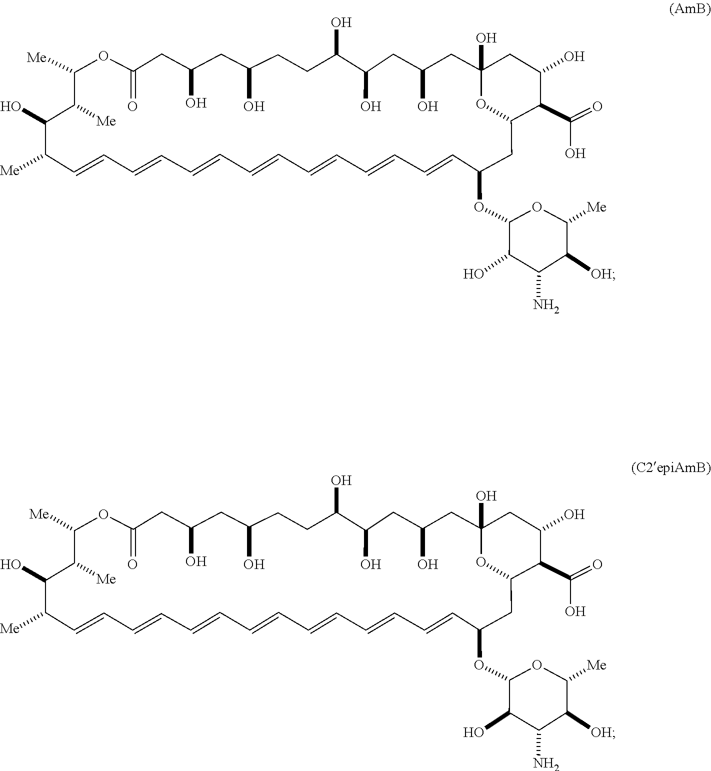
a compound having the structure of formula (I): 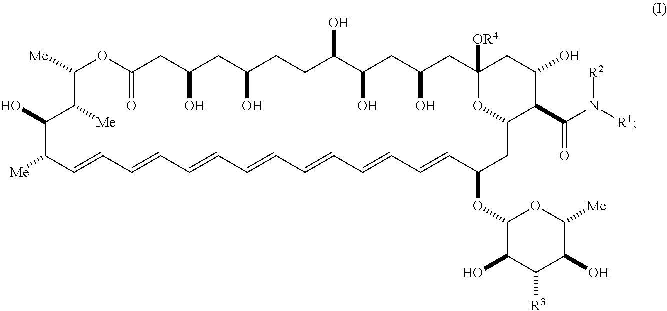
a compound having the structure of formula (II): 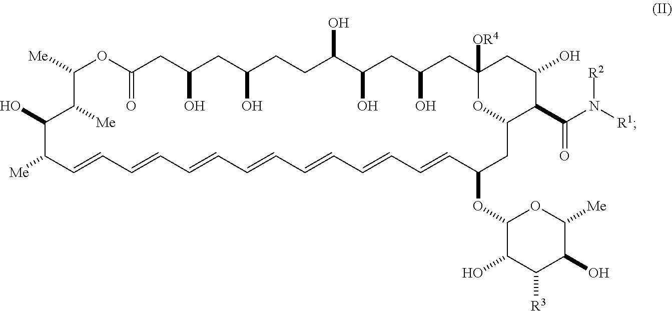
wherein:
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², taken together with the nitrogen to which they are attached, form a substituted or unsubstituted 3- to 10-membered heterocyclyl;
R³ is —NR⁵R⁶, substituted or unsubstituted amino, substituted or unsubstituted urea, substituted or unsubstituted carbamate or substituted or unsubstituted guanidinyl;
R⁴ is hydrogen or substituted or unsubstituted C₁₋₆ alkyl;
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, taken together with the nitrogen to which they are attached, form a substituted or unsubstituted 3- to 10-membered heterocyclyl; and
R^{f} is selected from the group consisting of 2-alken-1-yl, tert-butyl, benzyl and fluorenylmethyl.

2. The composition of claim 1, wherein the compound is AmB.

3. The composition of claim 1, wherein the compound is C2′epiAmB.

4. The composition of claim 1, wherein the compound is a compound having the structure of formula (I).

5. The composition of claim 1, wherein the compound is a compound having the structure of formula (II).

6. The composition of claim 1, wherein the compound is a compound having the structure of formula (I) or formula (II); and
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

7. The composition of claim 1 or 6, wherein the compound is a compound having the structure of formula (I) or formula (II); and
R¹ and R² independently are hydrogen, unsubstituted C₁₋₆ alkyl, hydroxyl C₁₋₆ alkyl, alkoxy C₁₋₆ alkyl, halo C₁₋₆ alkyl, amino C₁₋₆ alkyl, heterocyclyl C₁₋₆ alkyl, unsubstituted C₂₋₆ alkynyl, unsubstituted C₃₋₁₀ carbocyclyl, amino C₃₋₁₀ carbocyclyl, unsubstituted 3- to 10-membered heterocyclyl, or hydroxyl 3- to 10-membered heterocyclyl.

8. The composition of any one of claims 1 and 6-7, wherein the compound is a compound having the structure of formula (I) or formula (II); and
at least one of R¹ and R² is hydrogen.

9. The composition of any one of claims 1 and 6-8, wherein the compound is a compound having the structure of formula (I) or formula (II); and
R¹ and R² are not both hydrogen.

10. The composition of any one of claims 1 and 6-7, wherein the compound is a compound having the structure of formula (I) or formula (II); and
R¹ and R², taken together with the nitrogen to which they are attached, form a substituted or unsubstituted 3- to 10-membered heterocyclyl.

11. The composition of any one of claims 1 and 6-10, wherein the compound is a compound having the structure of formula (I) or formula (II); R³ is —NR⁵R⁶;
R⁵ and R⁶ independently are hydrogen, C(O)OR, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, taken together with the nitrogen to which they are attached, form a substituted or unsubstituted 3- to 10-membered heterocyclyl; and
R^{f} is selected from the group consisting of 2-alken-1-yl, tert-butyl, benzyl and fluorenylmethyl.

12. The composition of claim 11, wherein R⁵ and R⁶ independently are hydrogen, C(O)OR, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

13. The composition of claim 12, wherein R⁵ and R⁶ independently are hydrogen or C(O)OR, optionally wherein R^{f} is fluorenylmethyl.

14. The composition of claim 11, wherein at least one of R⁵ and R⁶ is hydrogen.

15. The composition of claim 11, wherein R⁵ and R⁶ are both hydrogen.

16. The composition of any one of claims 1 and 6-15, wherein the compound is a compound having the structure of formula (I) or formula (II); and
R⁴ is hydrogen, substituted or unsubstituted C₁₋₆ alkyl, or substituted or unsubstituted C₂₋₆ alkenyl.

17. The composition of claim 16, wherein R⁴ is hydrogen, halo C₁₋₆ alkyl, or unsubstituted C₂₋₆ alkenyl.

18. The composition of claim 17, wherein R⁴ is hydrogen.

19. The composition of claim 1, wherein the compound is selected from the group consisting of:

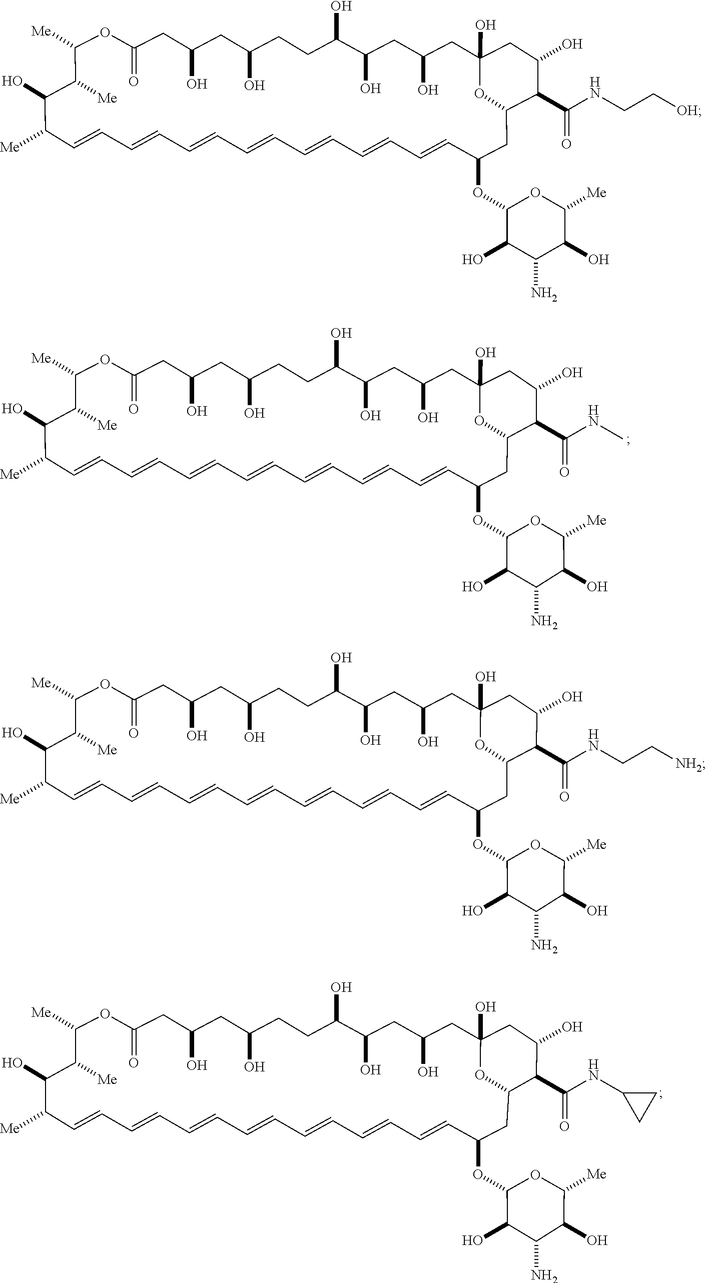

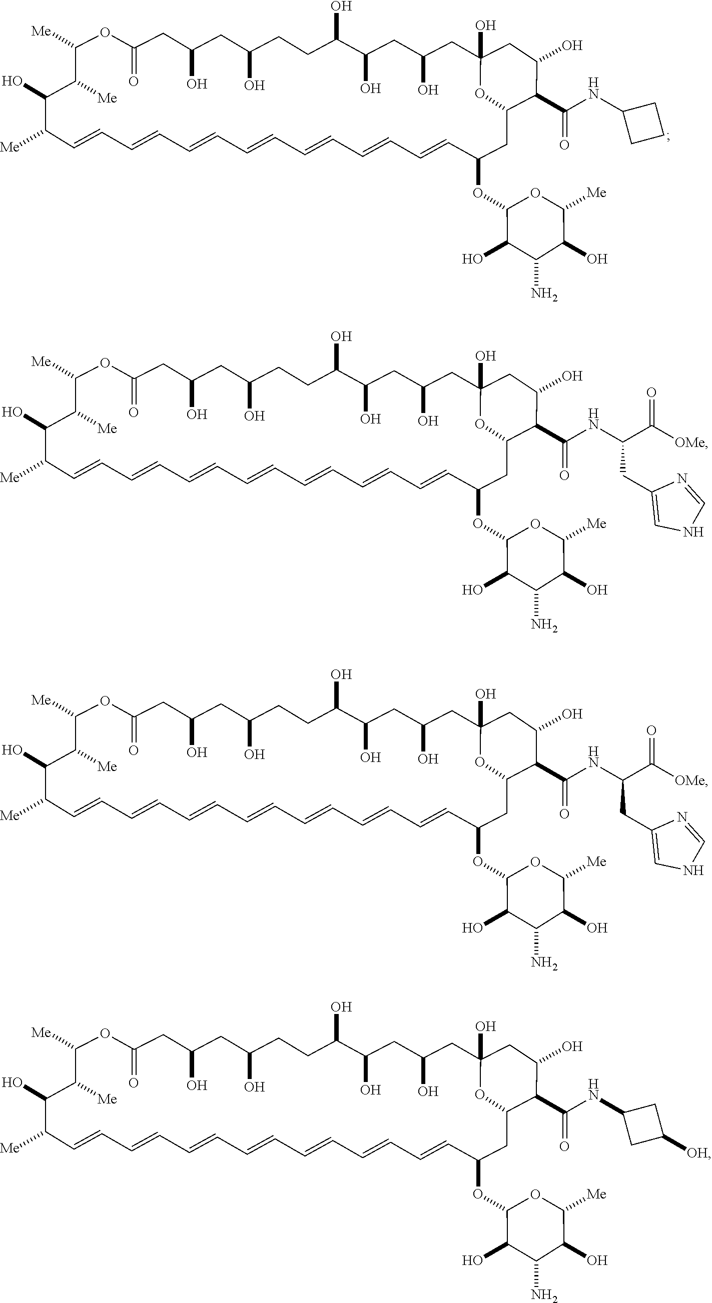

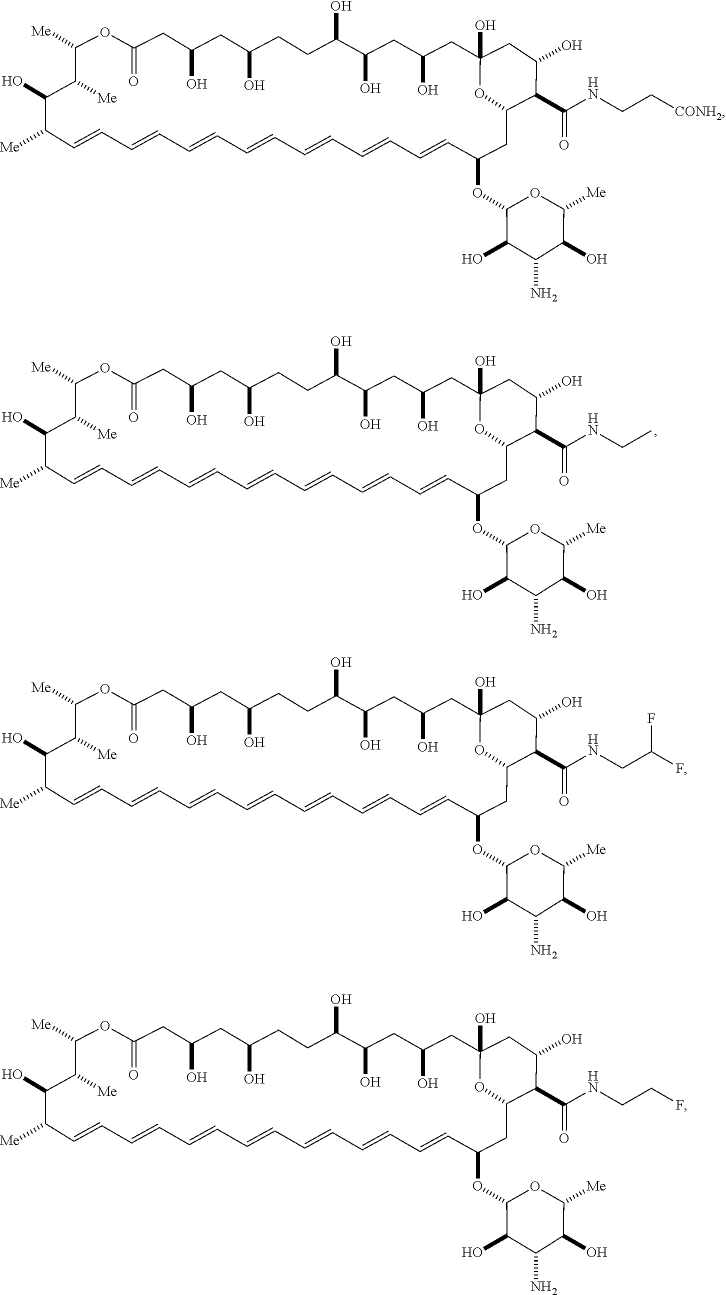

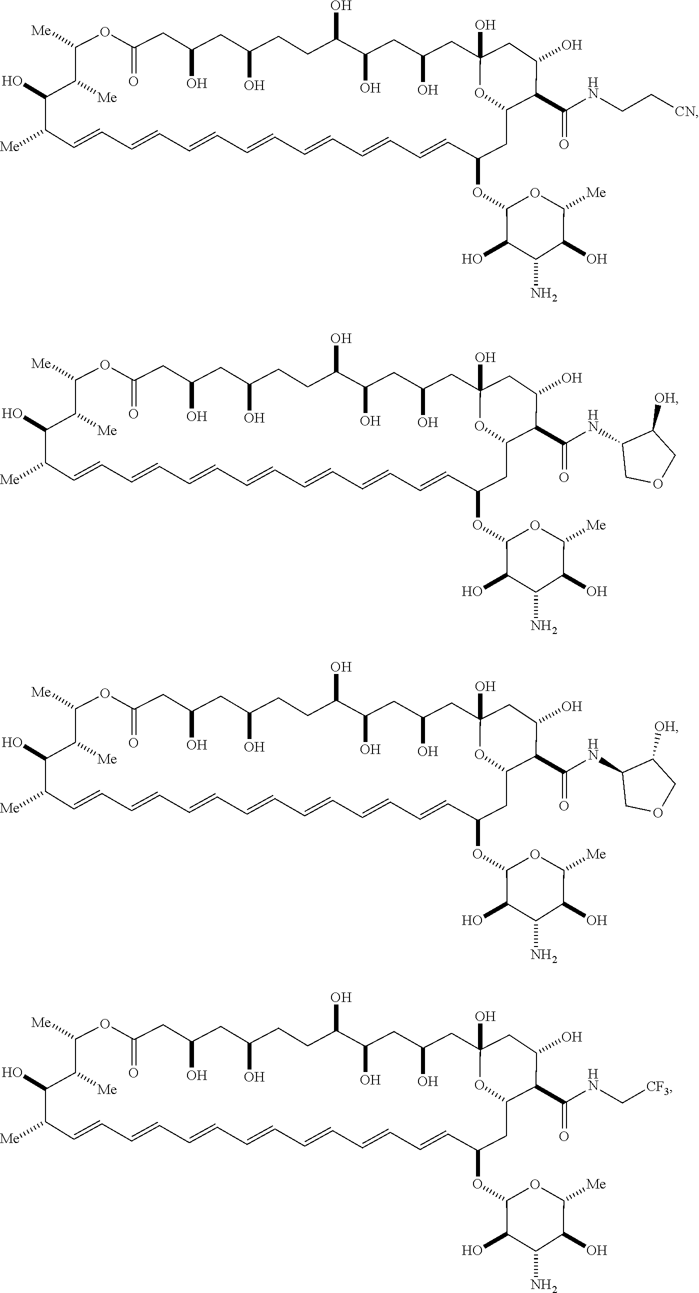

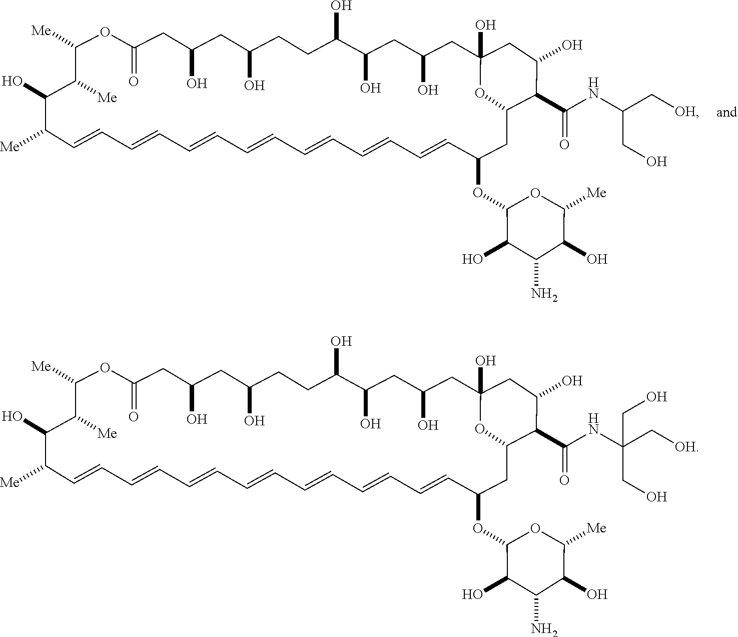 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)NC(CO)CO ; C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)NC(CO)(CO)CO]

20. The composition of claim 1, wherein the compound is selected from the group consisting of:

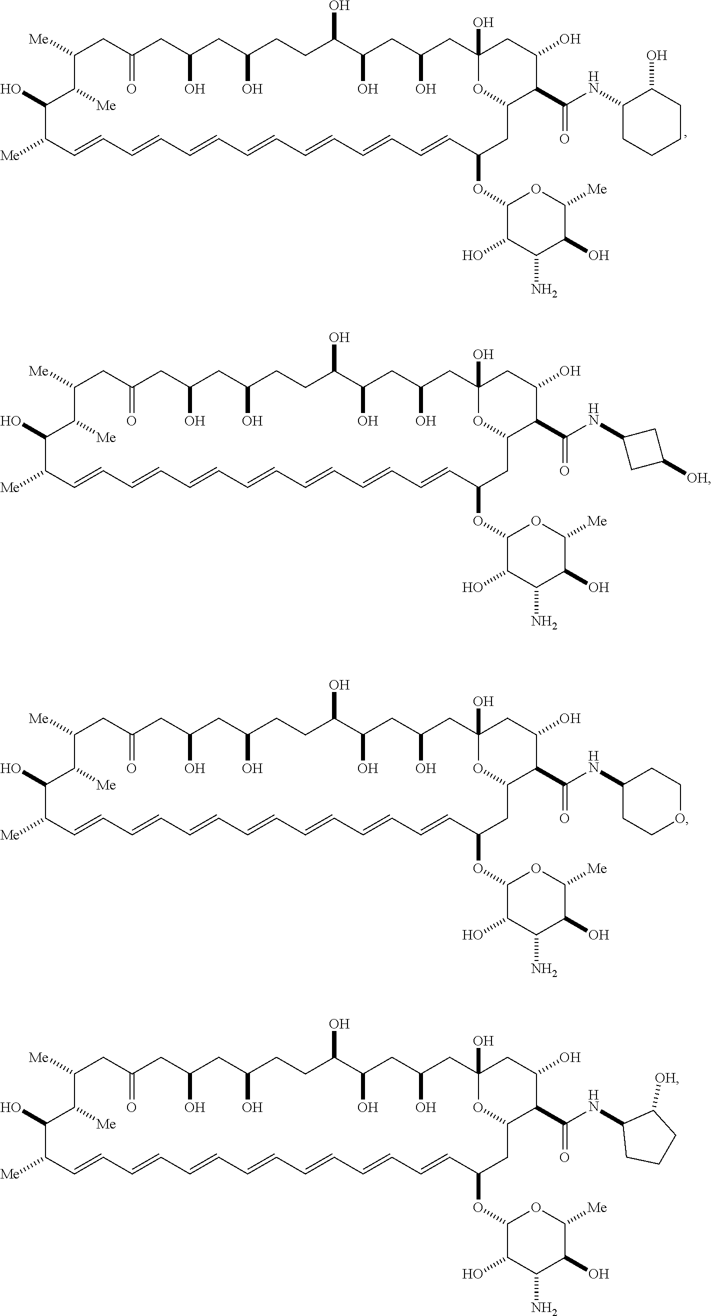

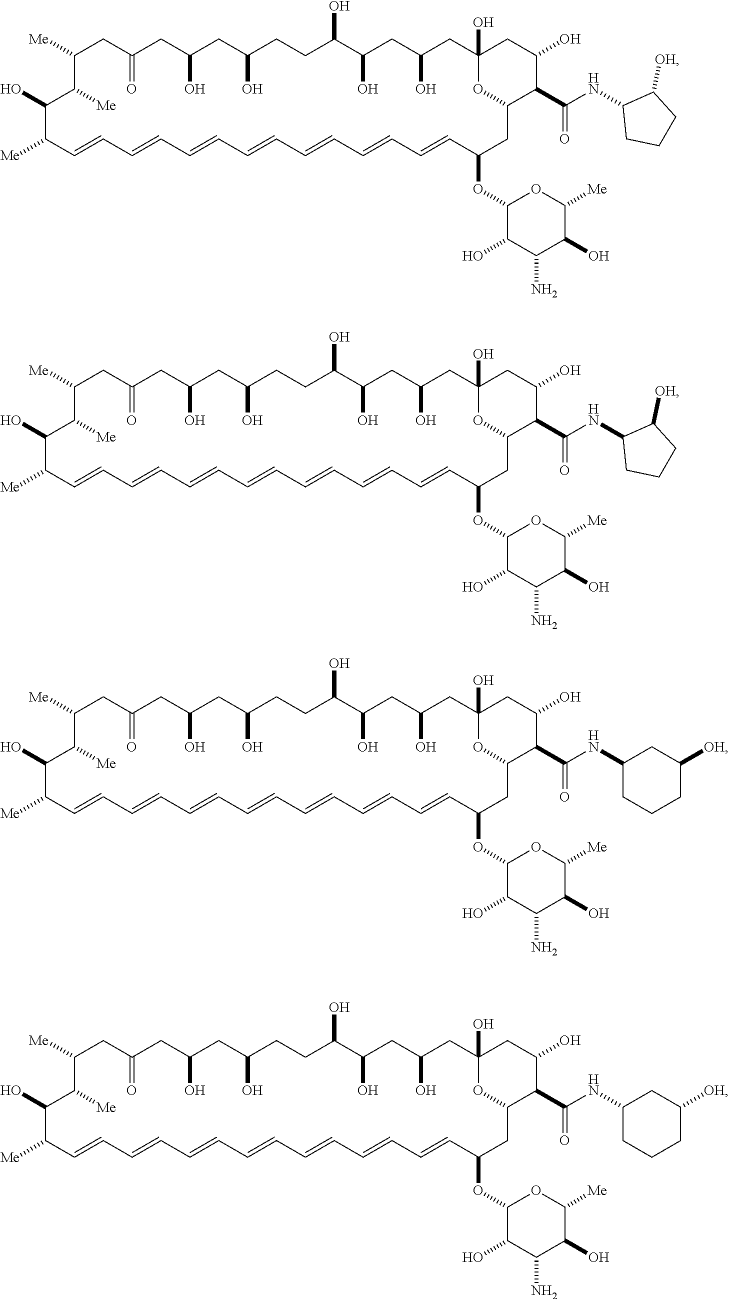

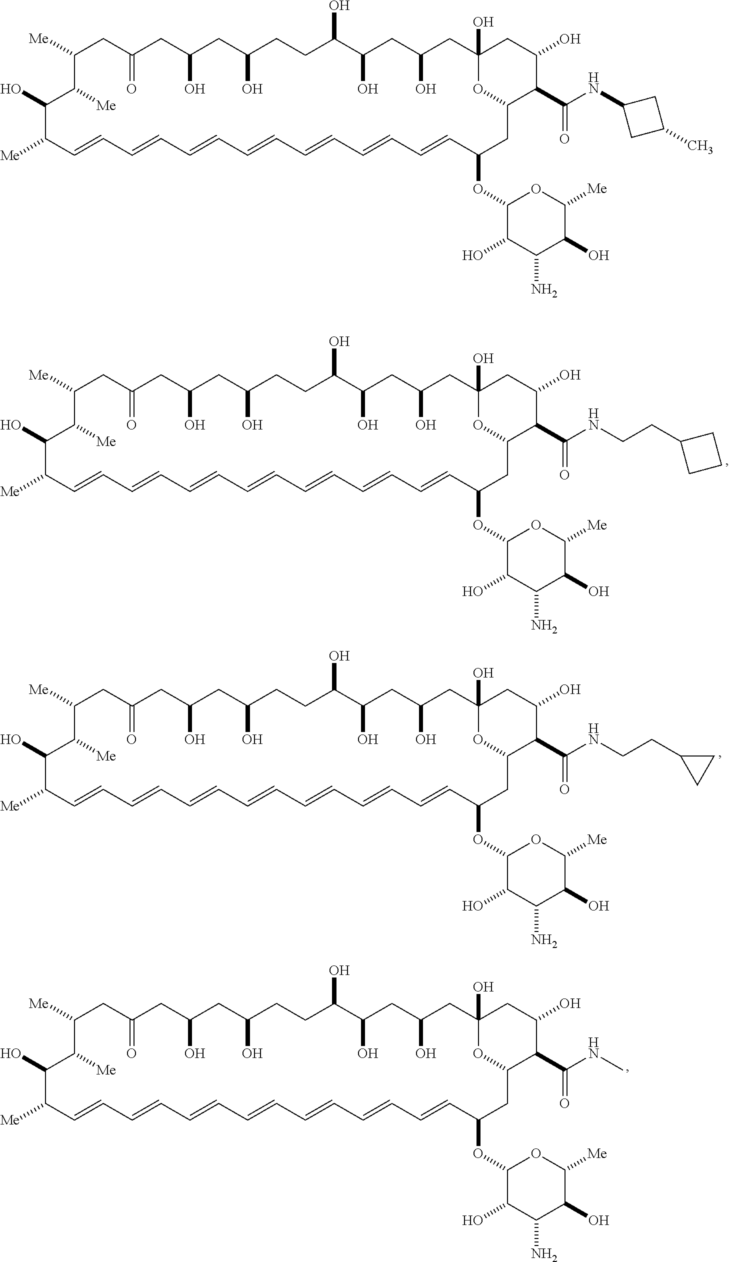

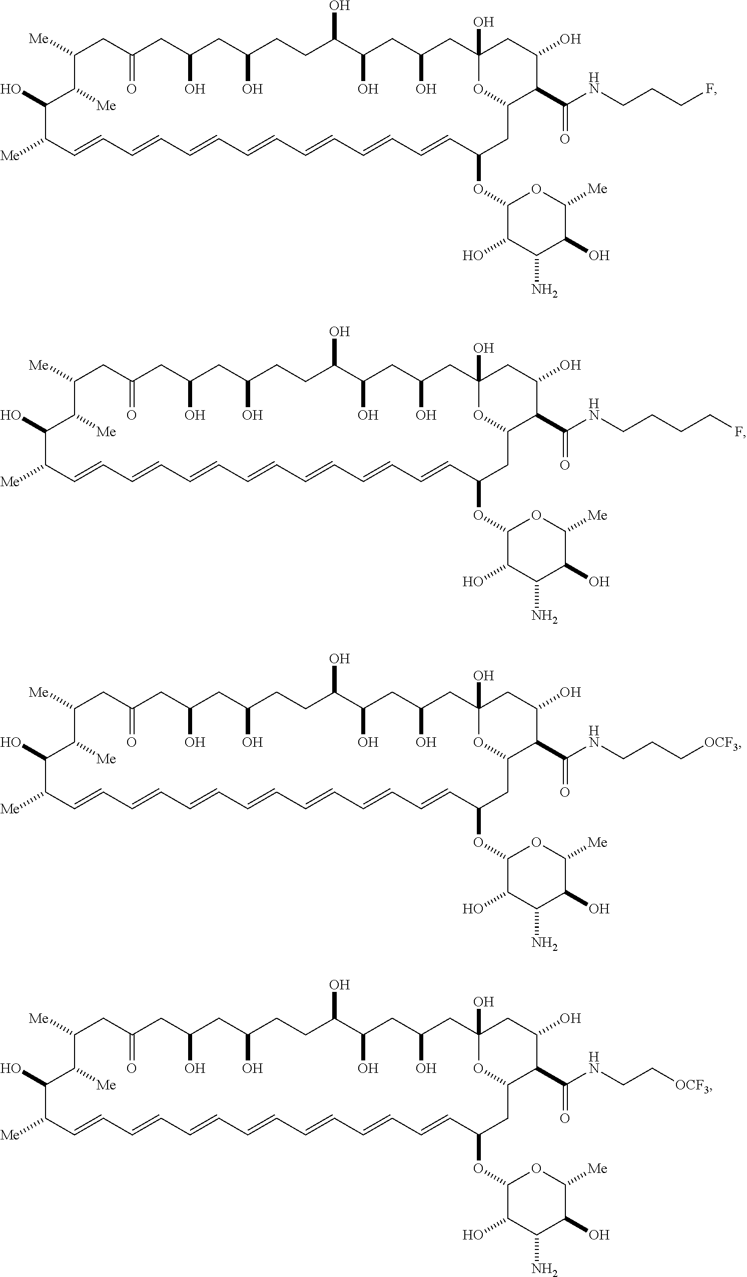

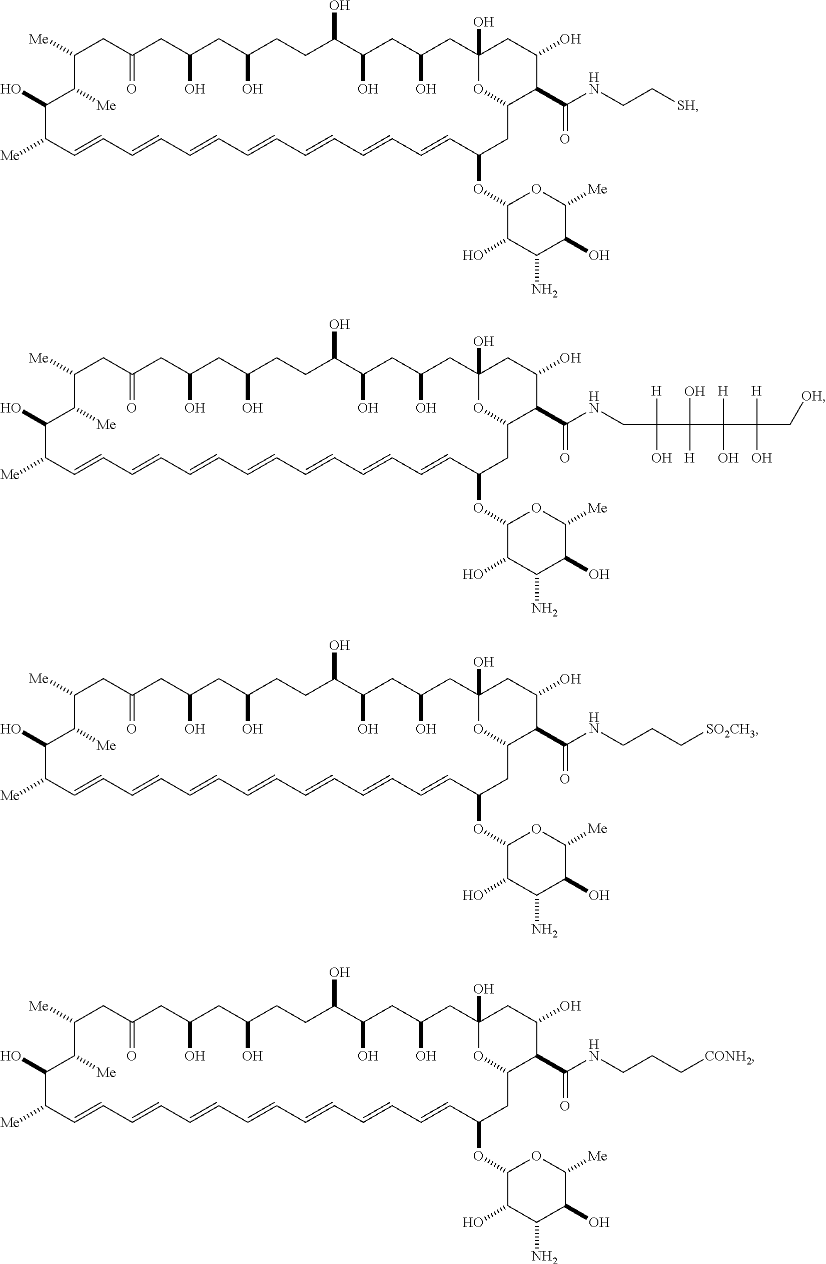

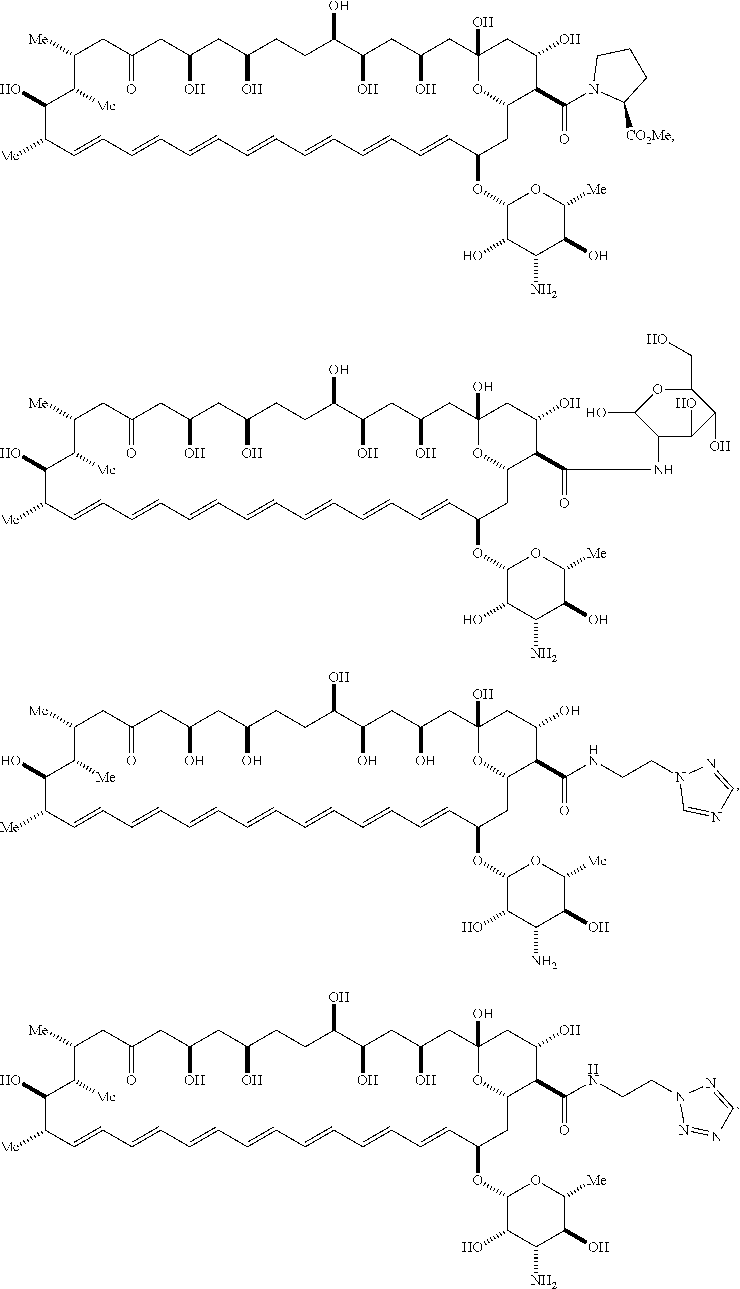

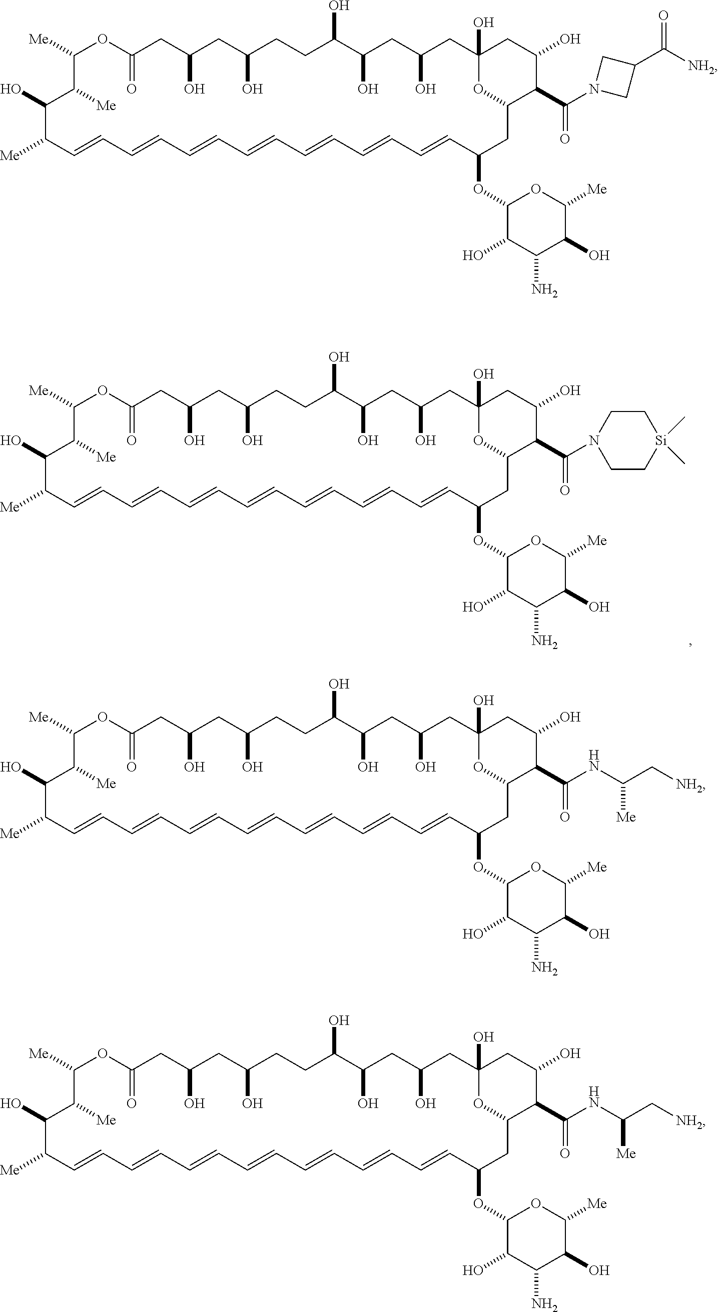

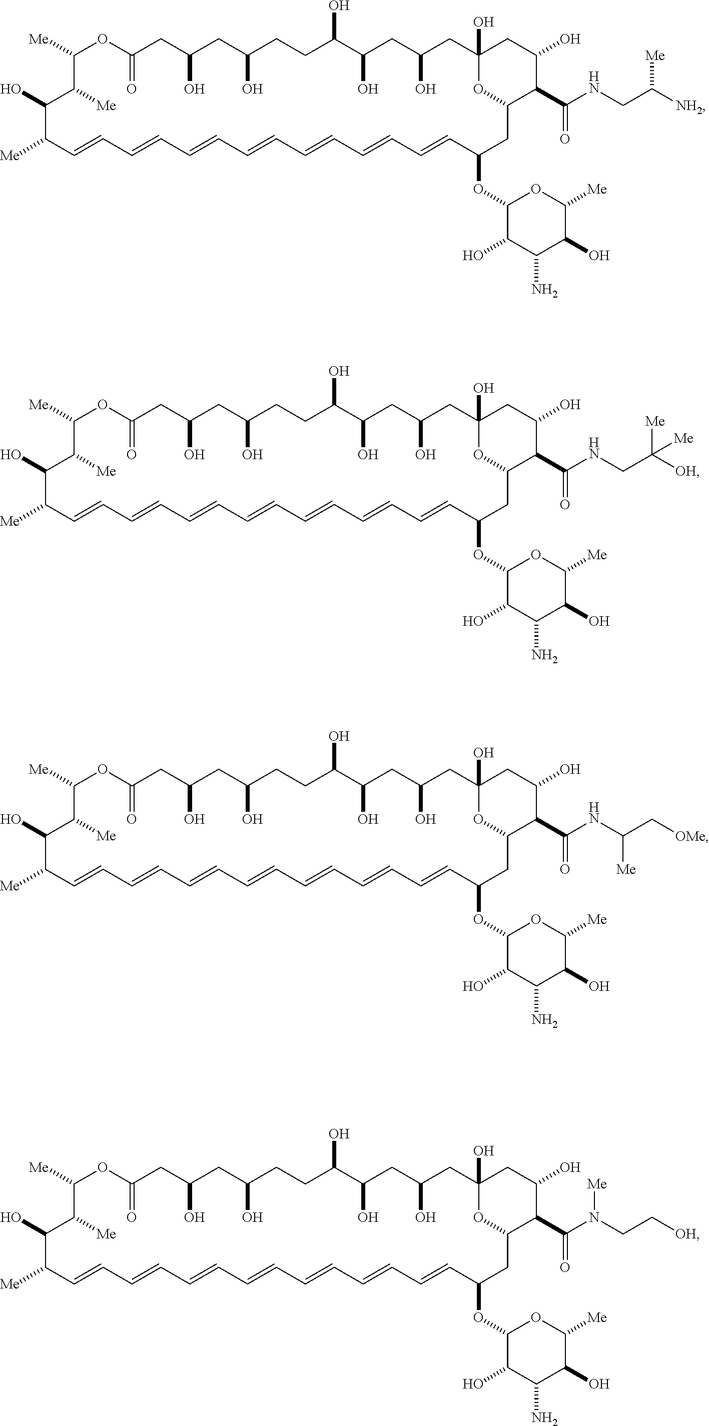

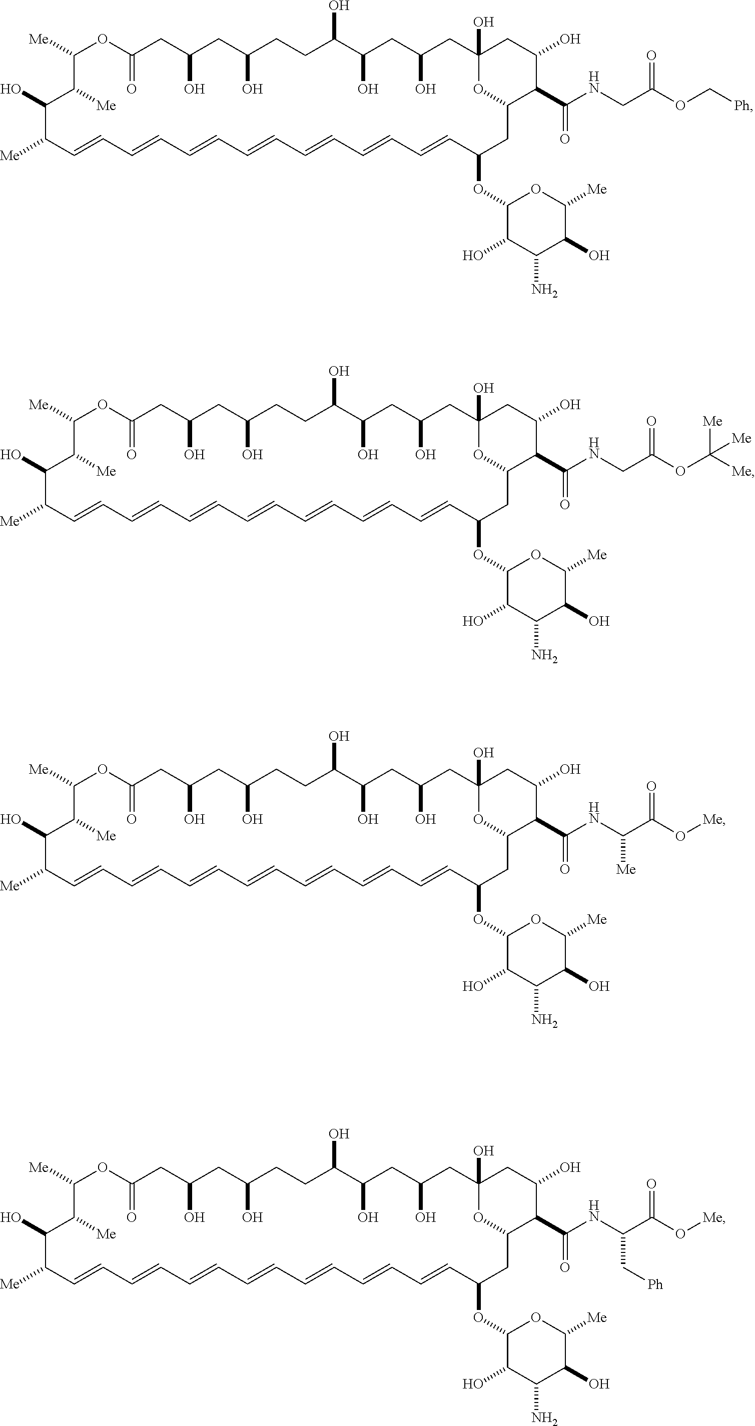

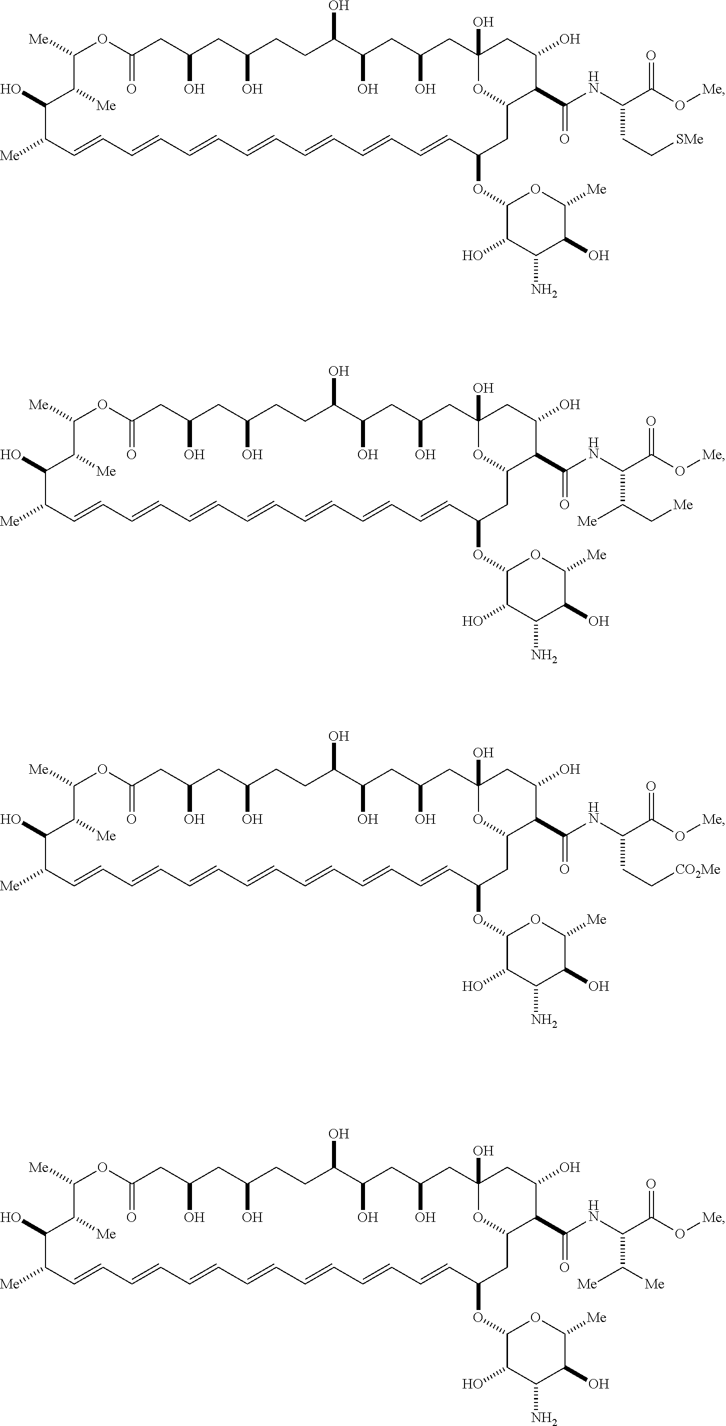

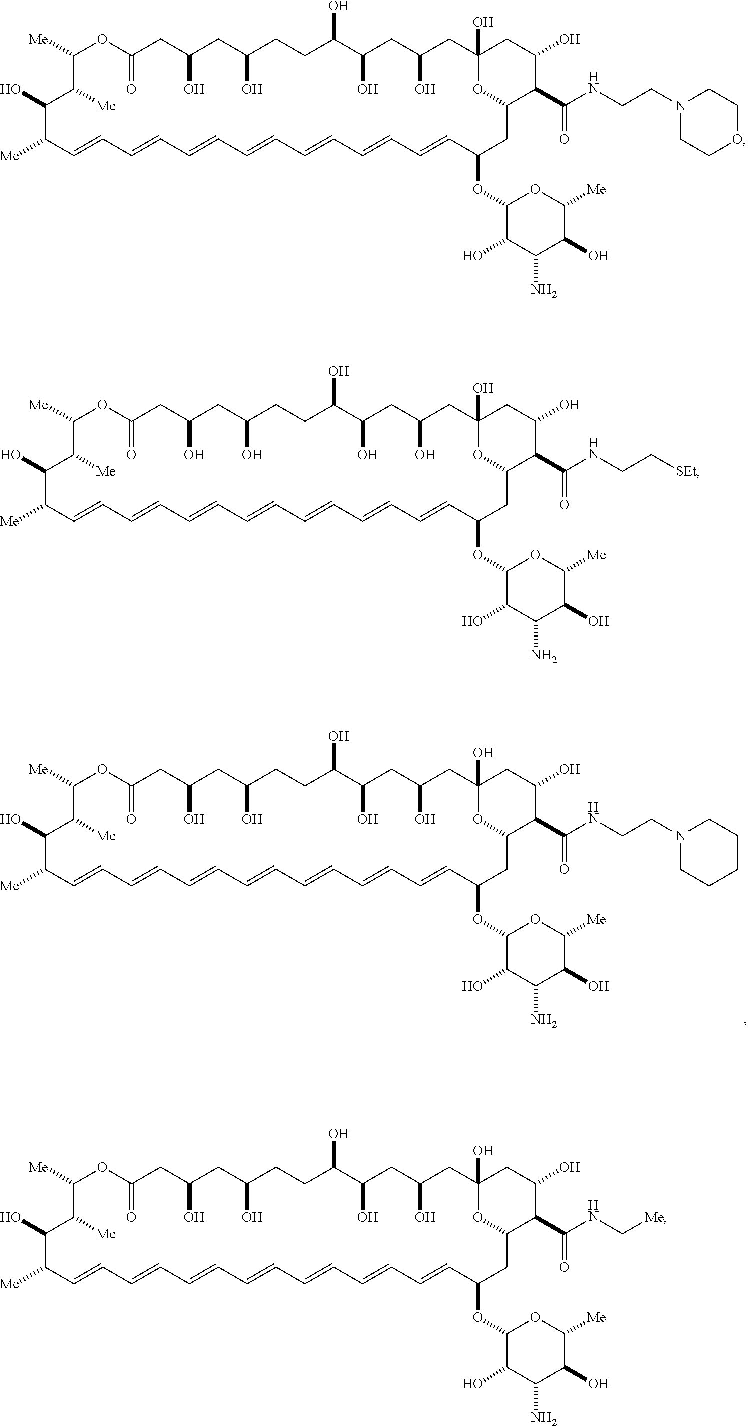

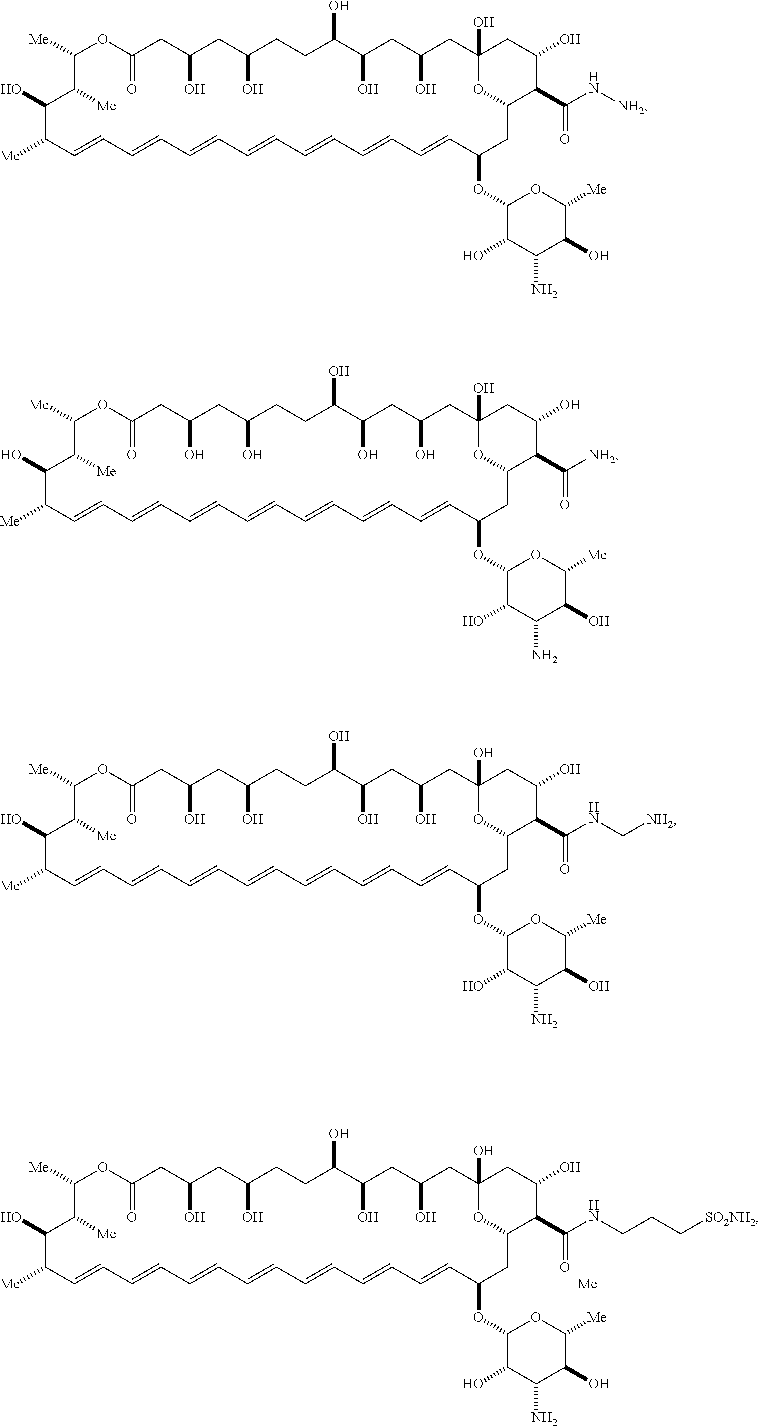

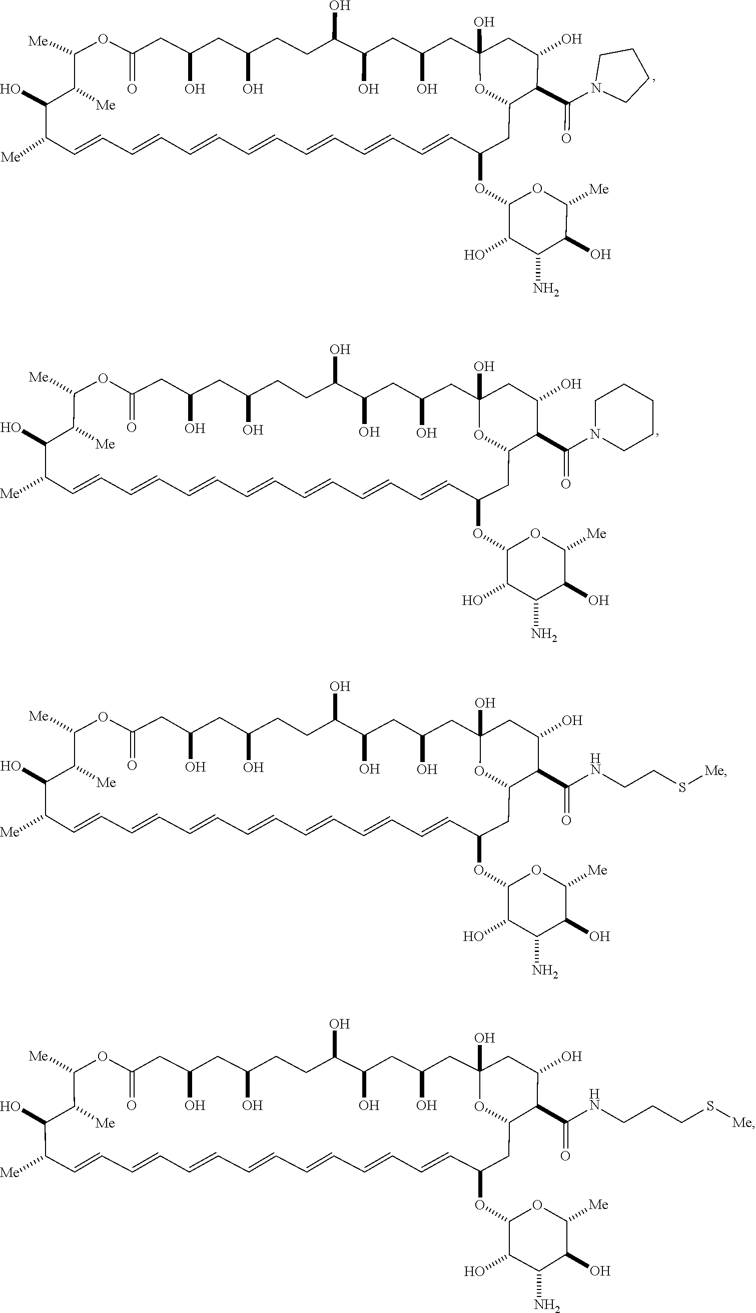

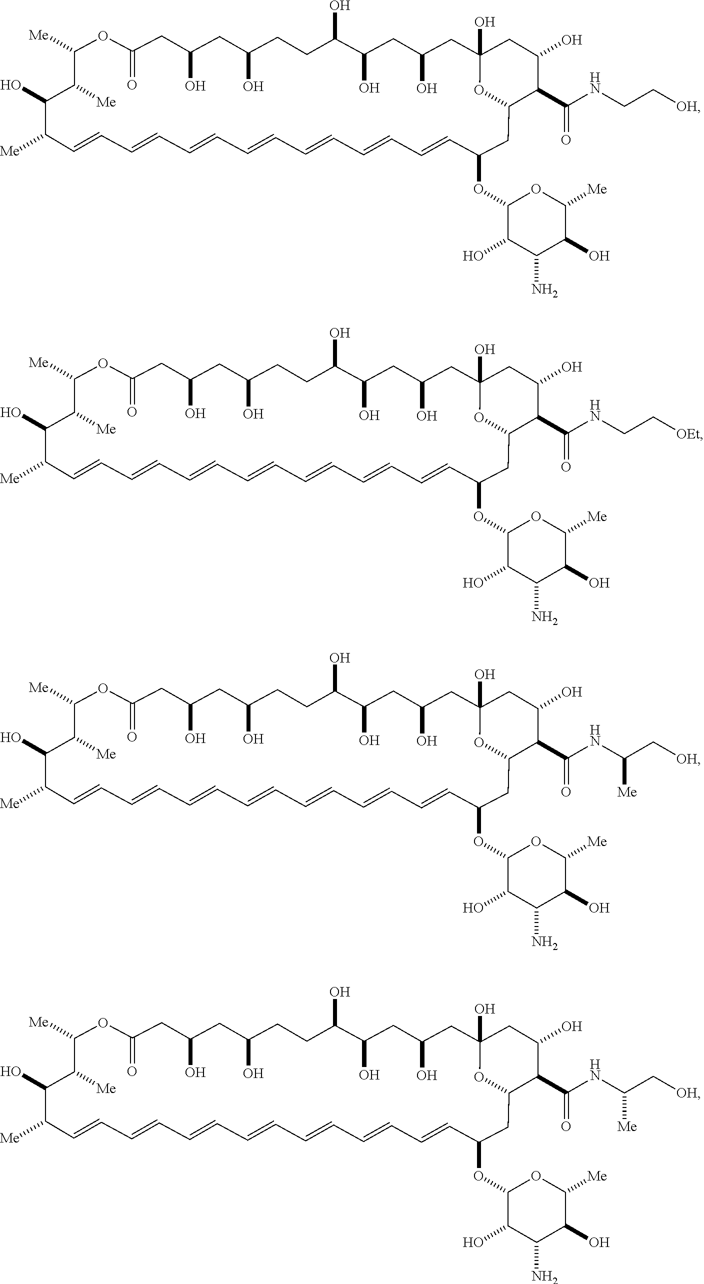

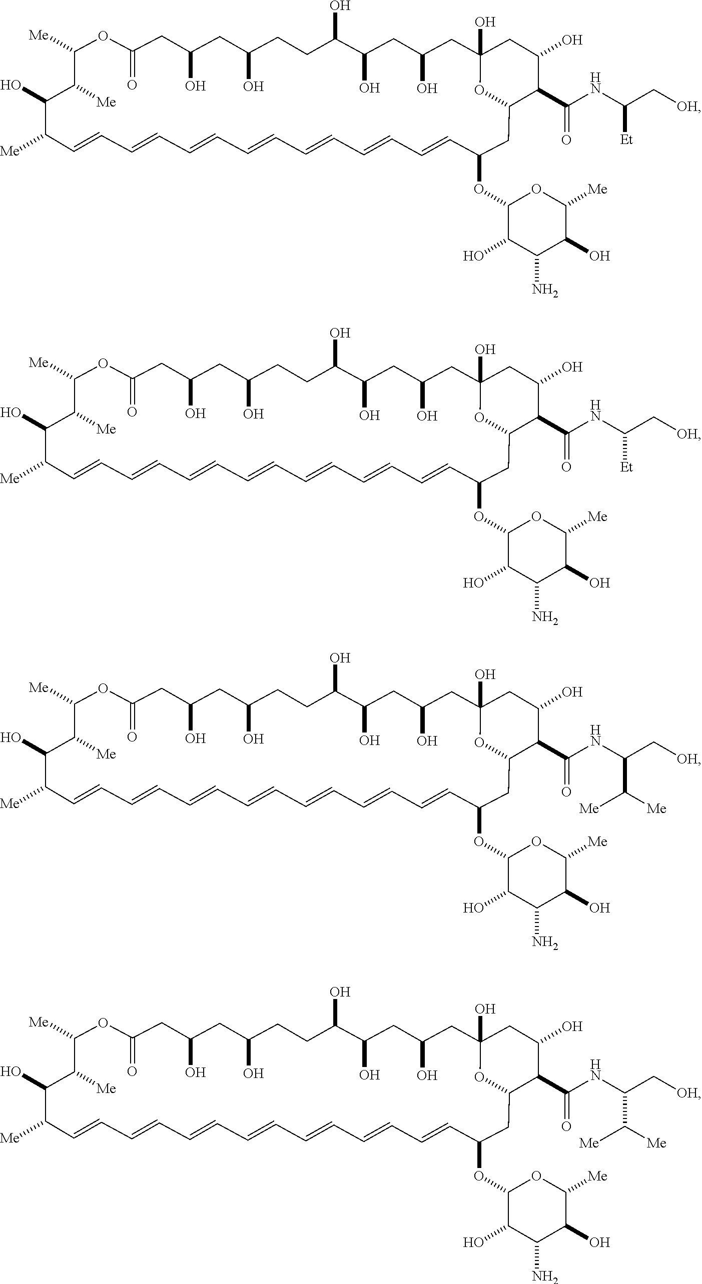

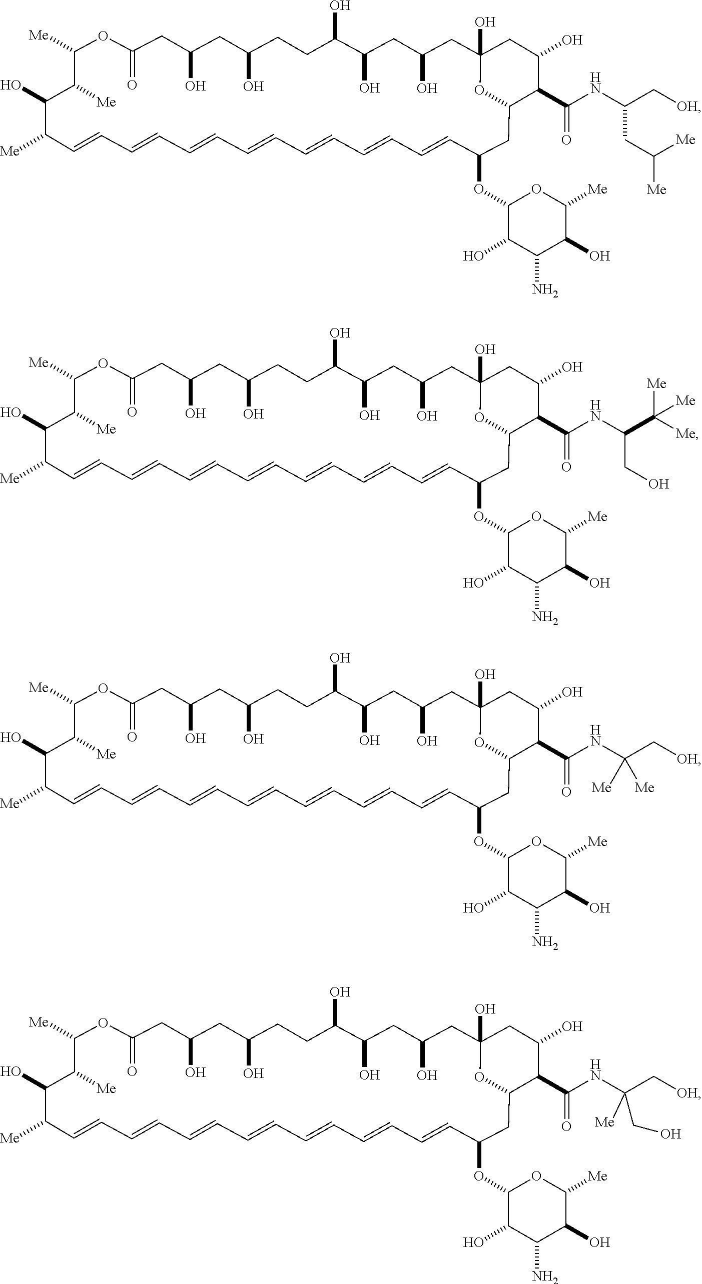

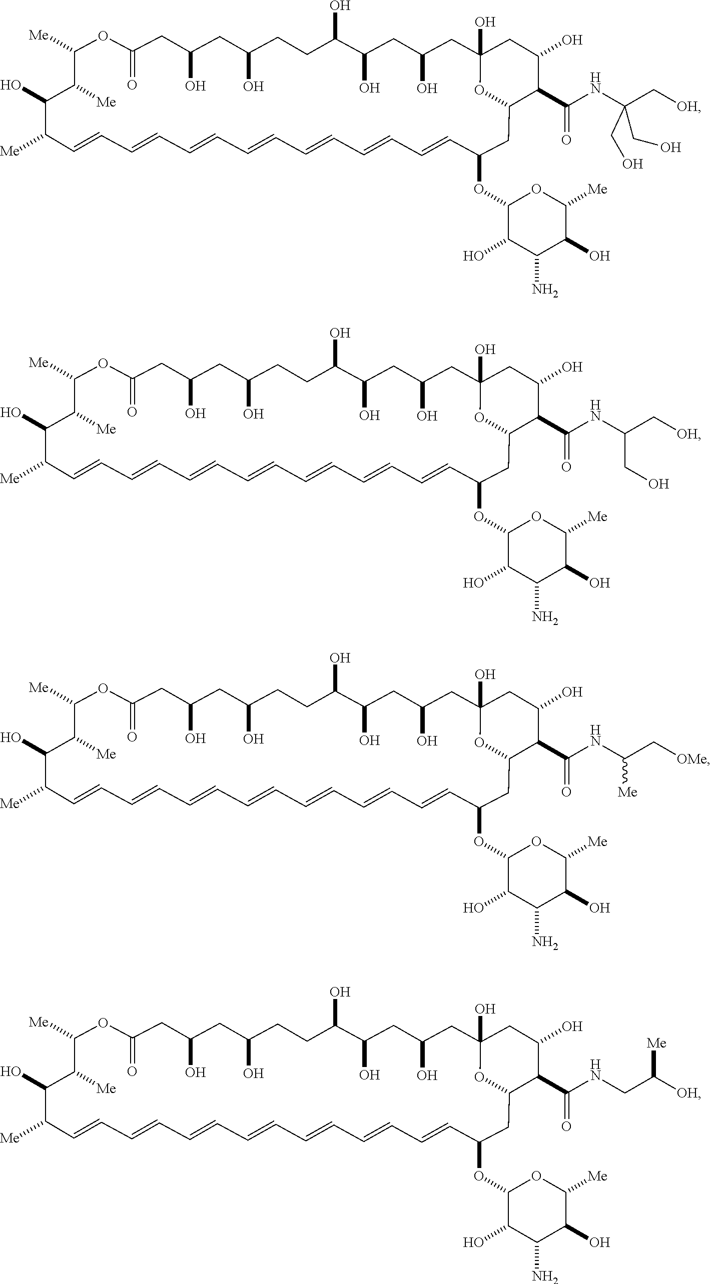

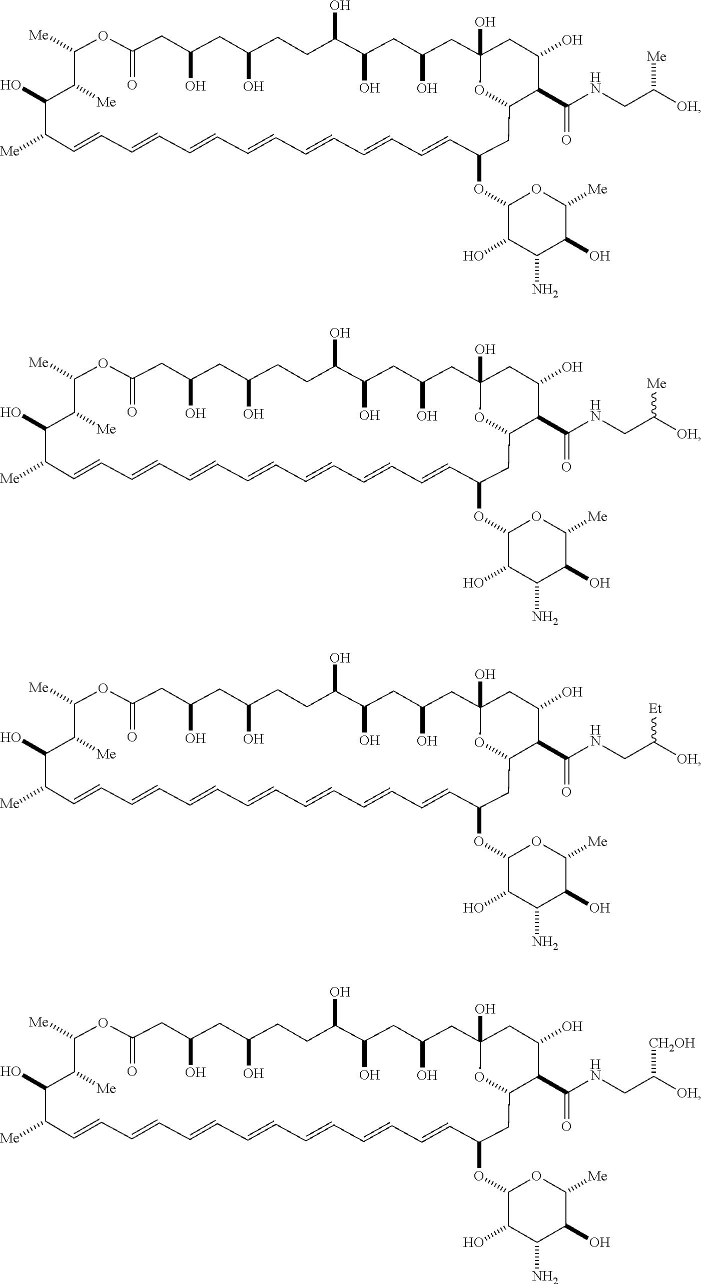

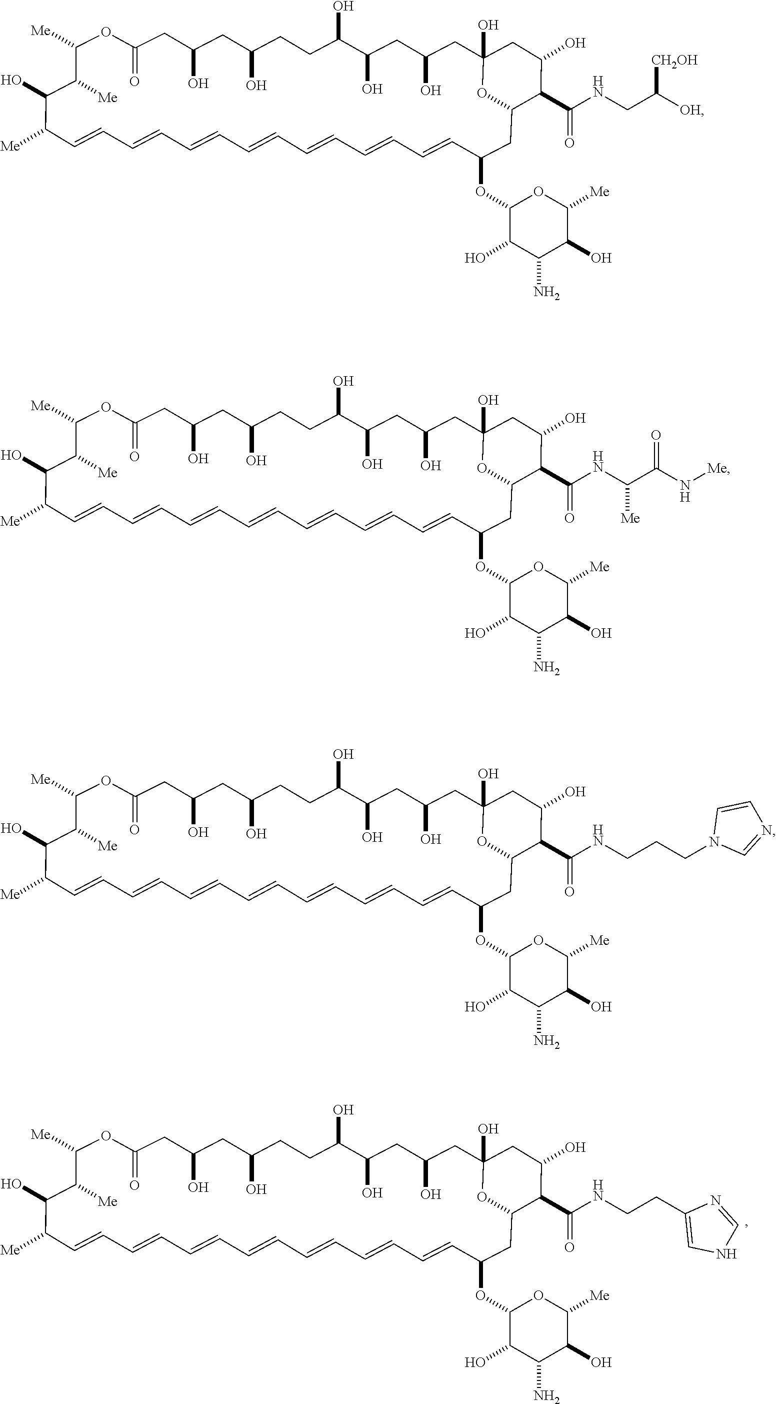

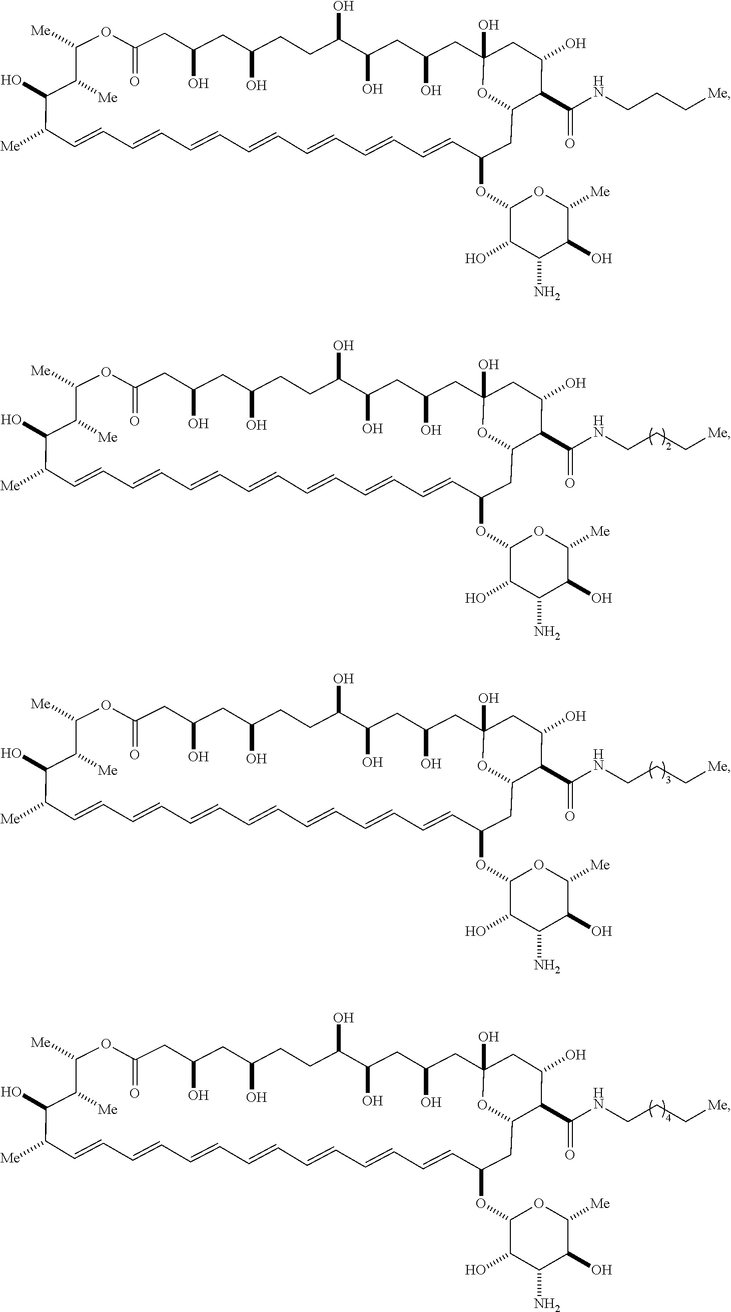

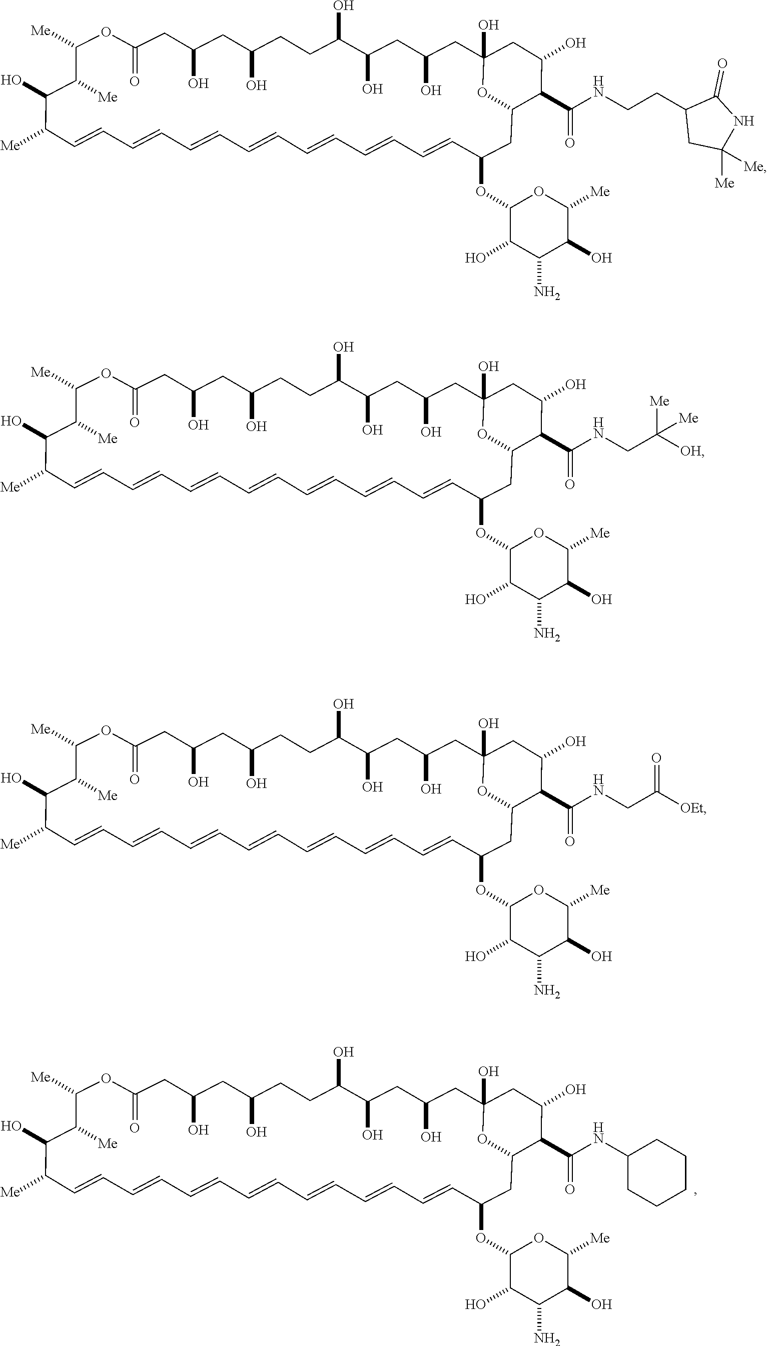

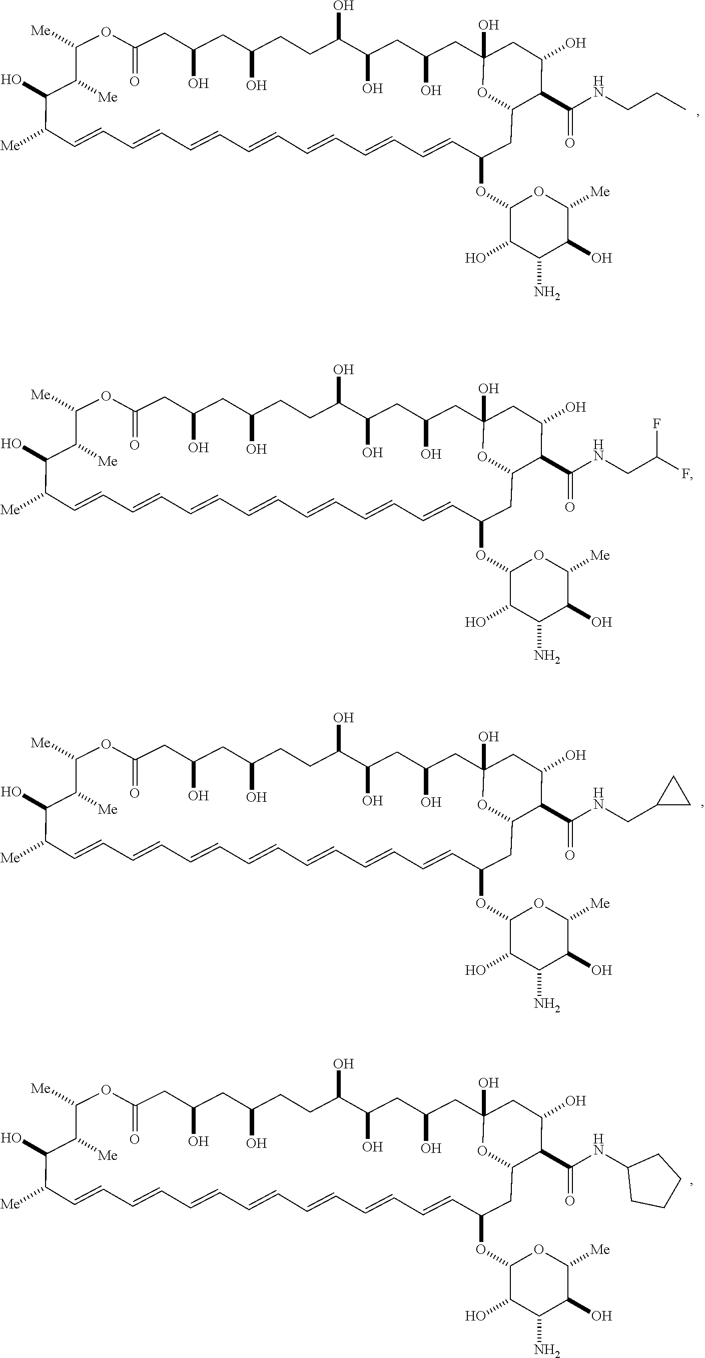

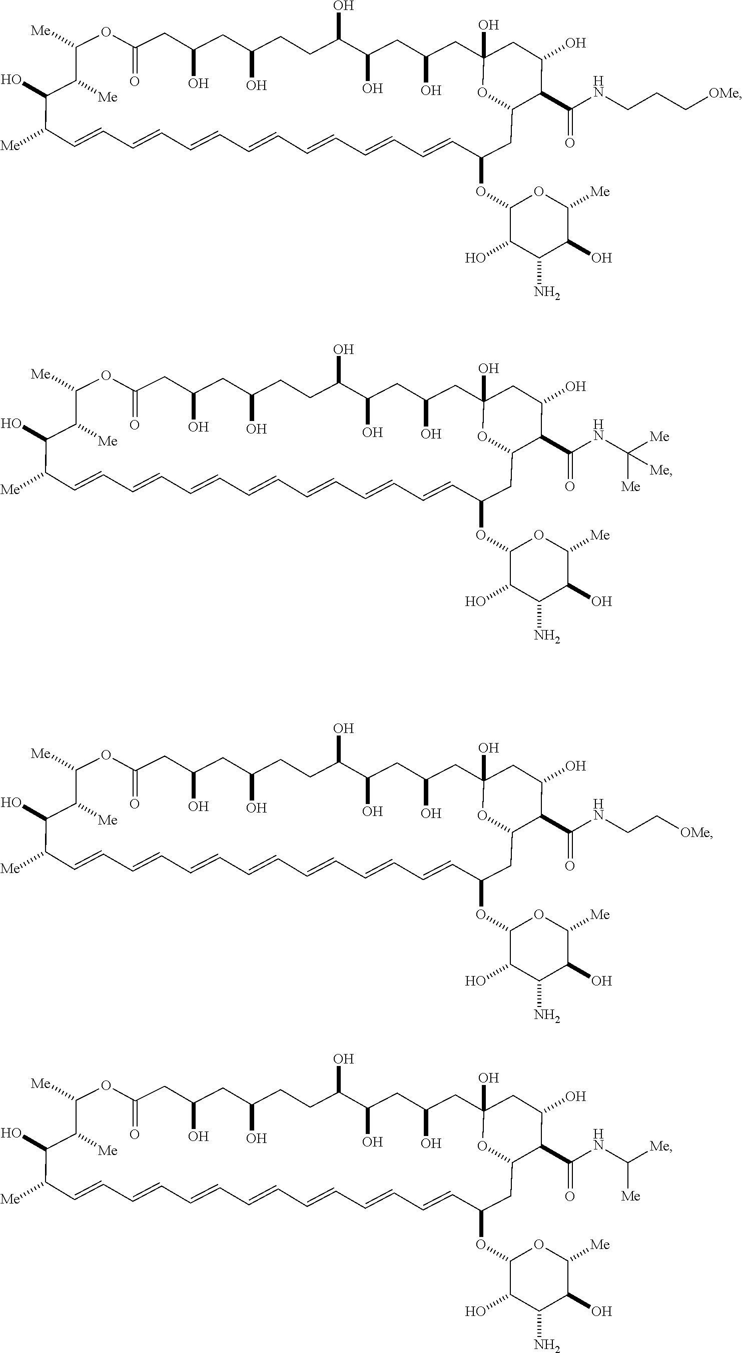

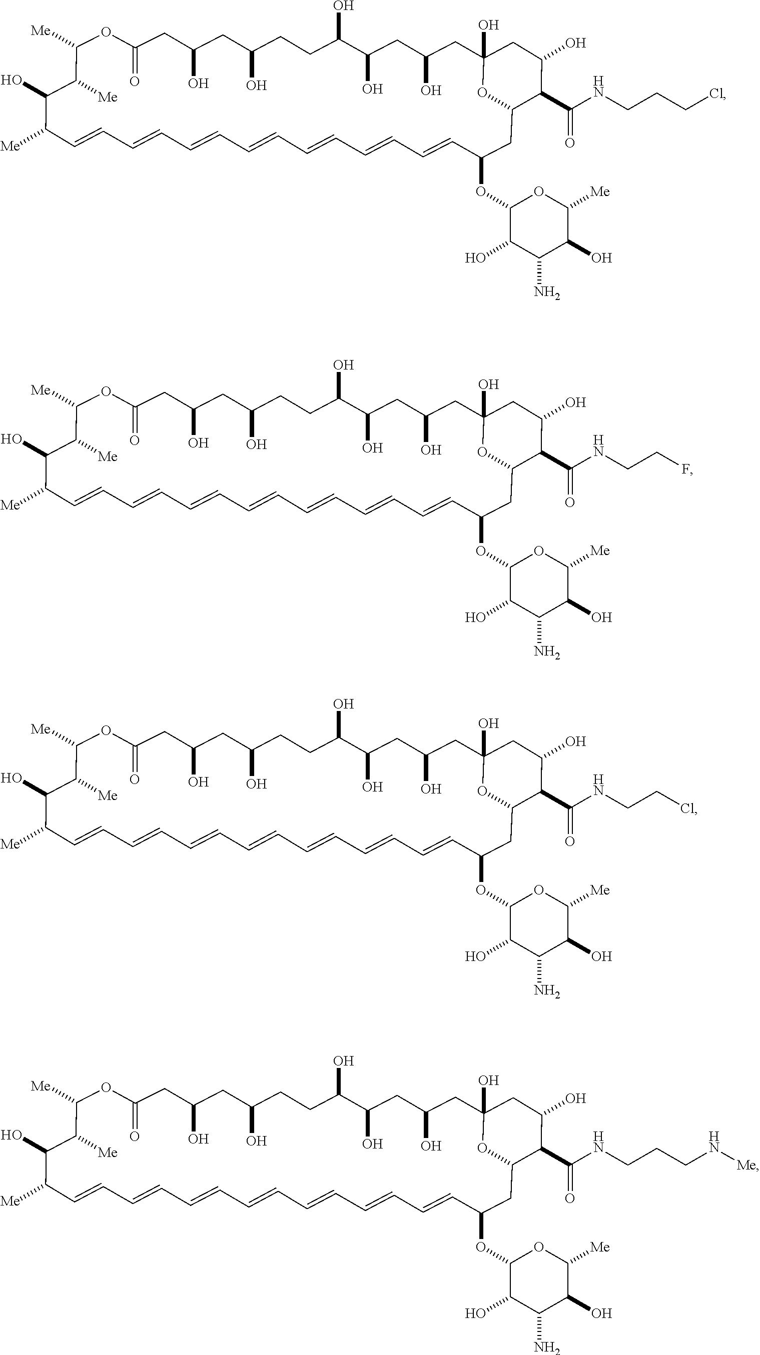

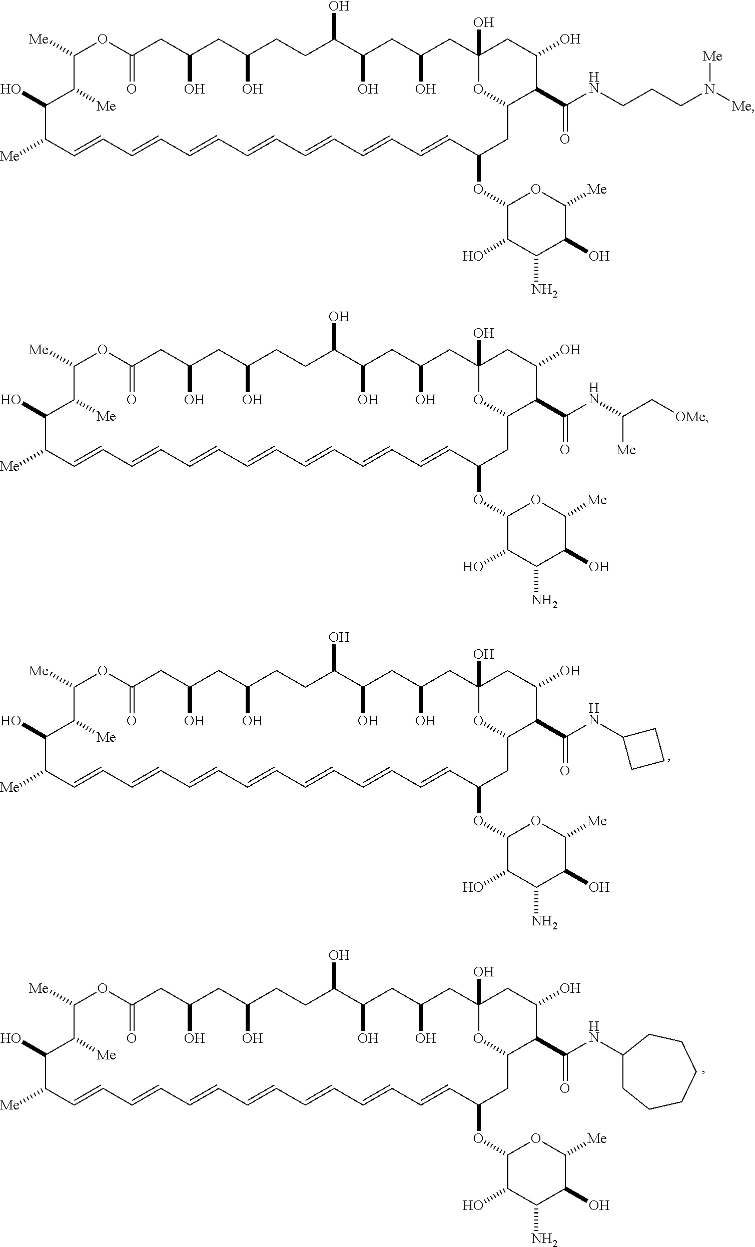

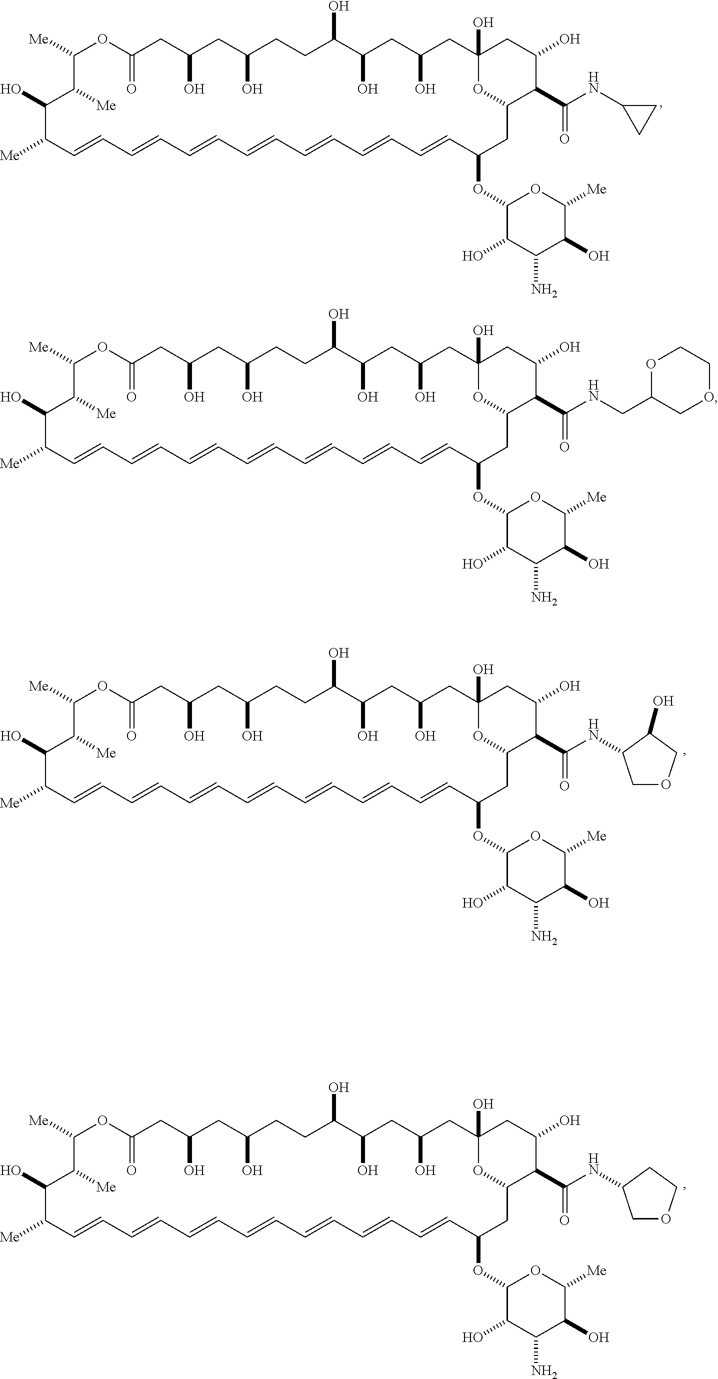

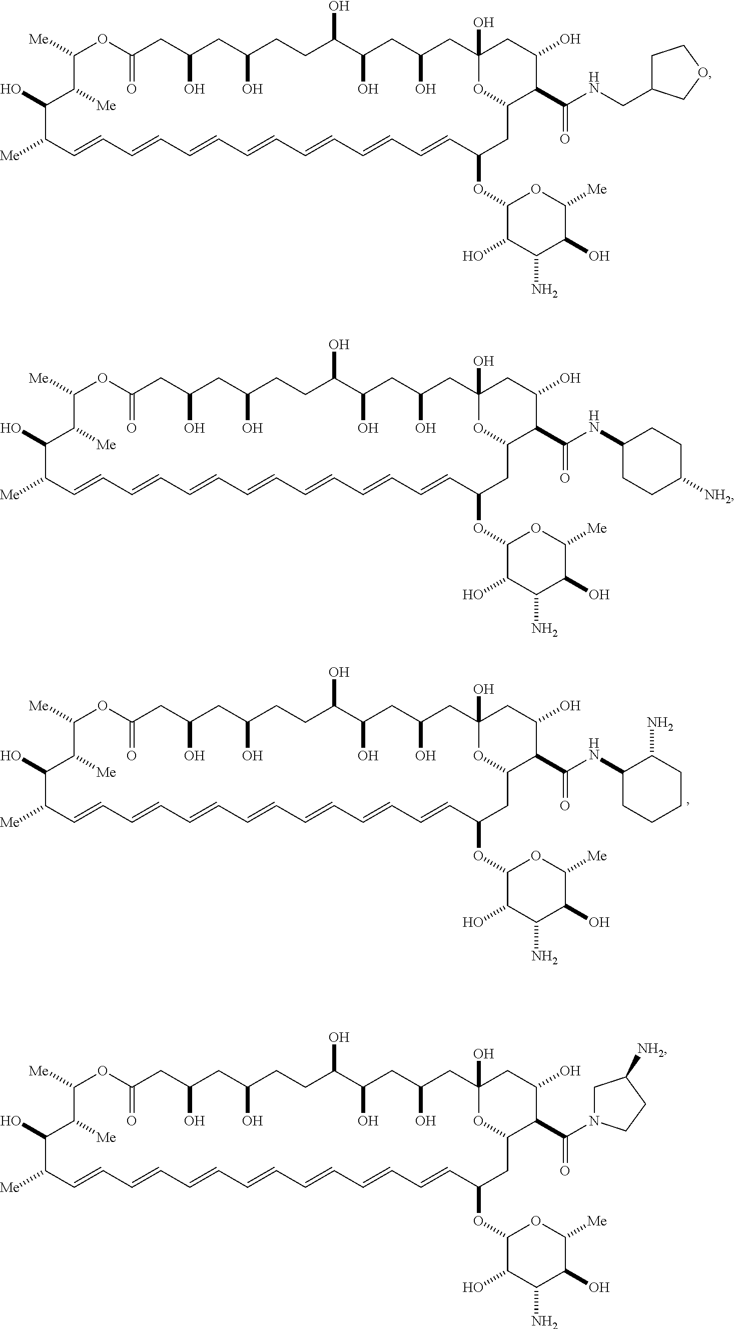

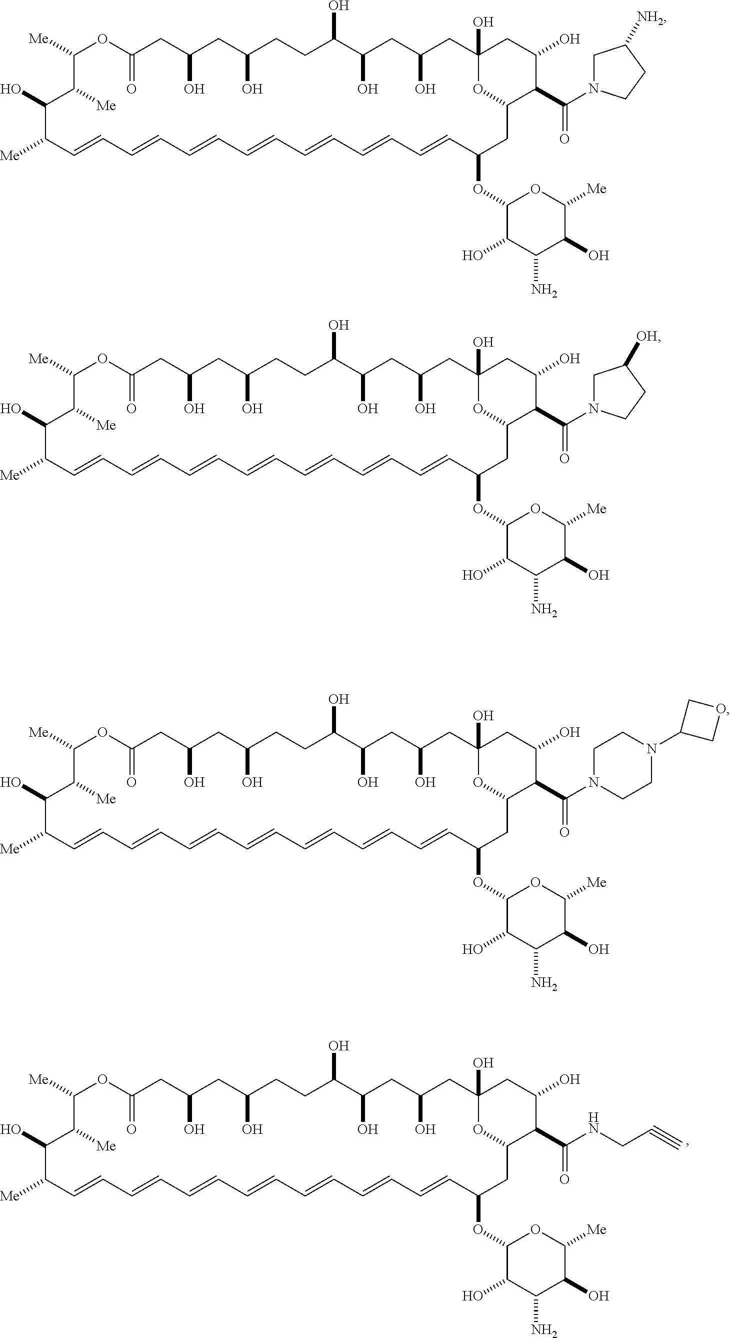

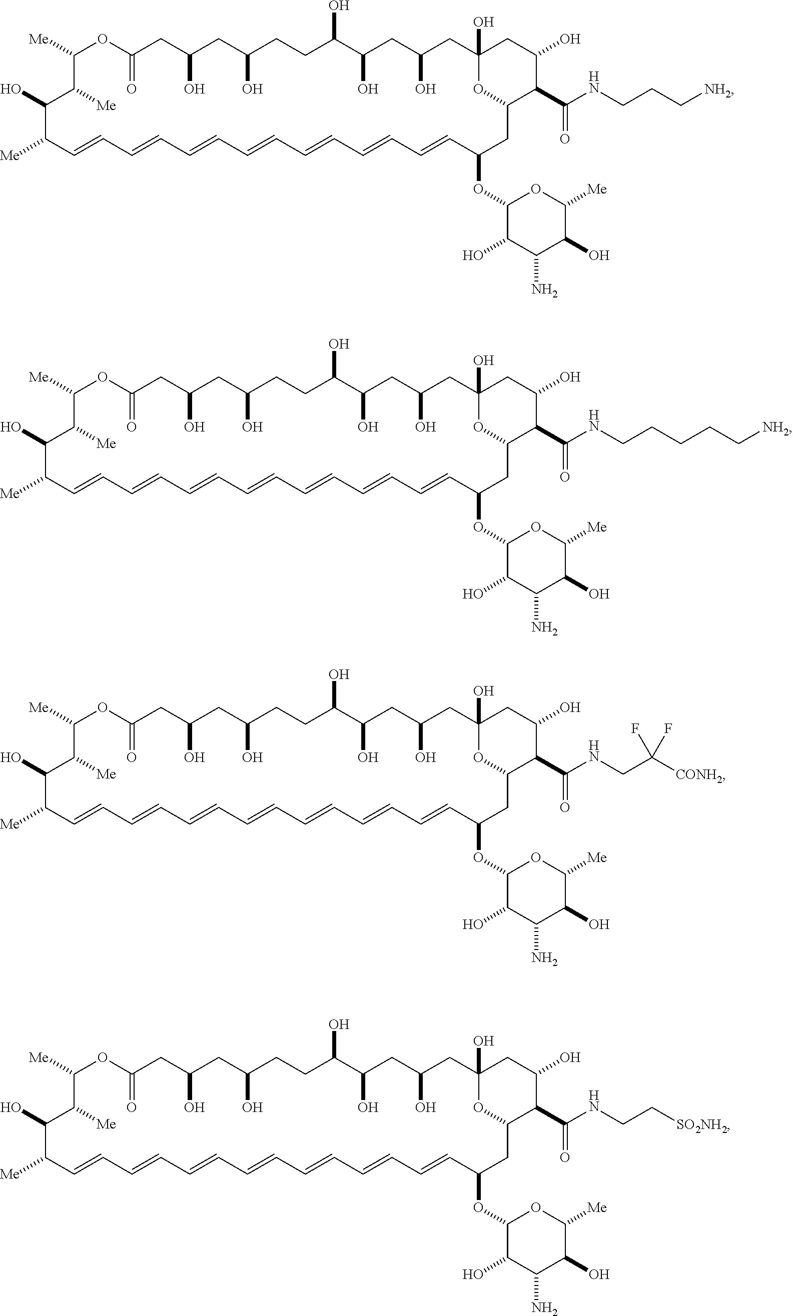

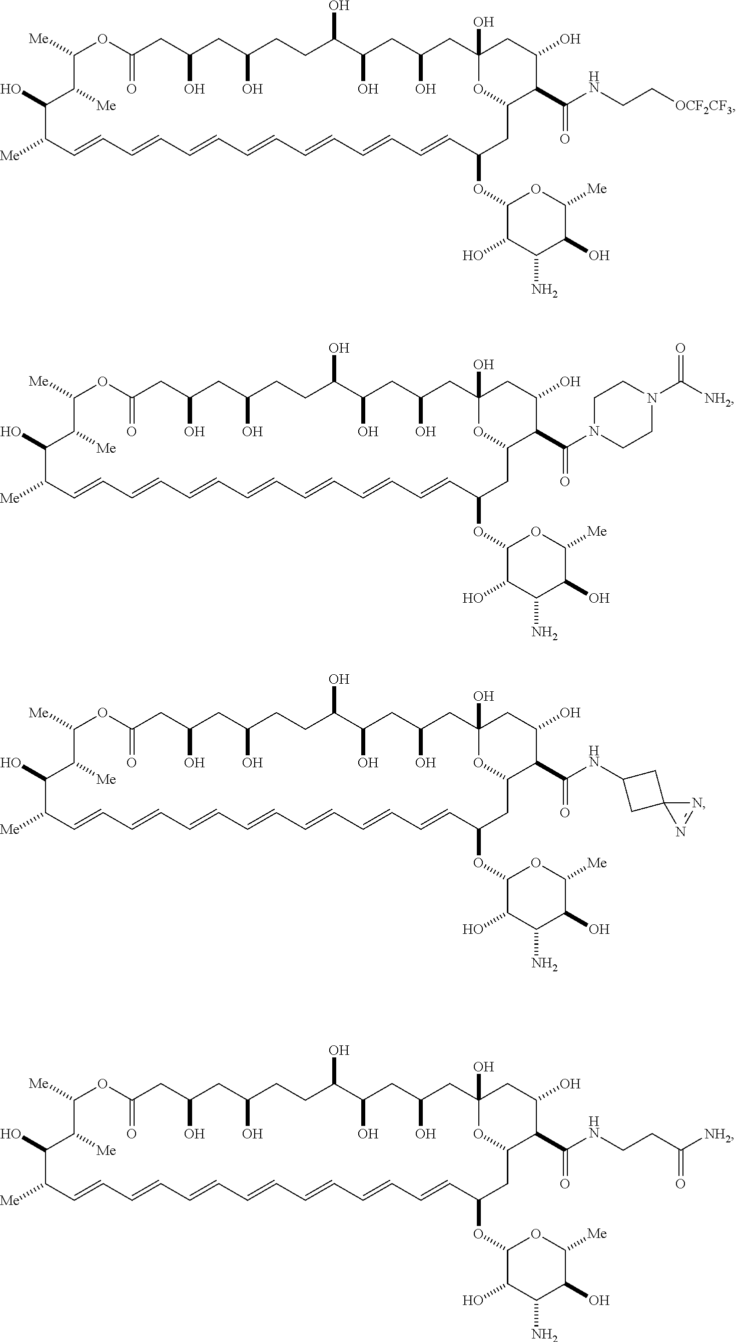

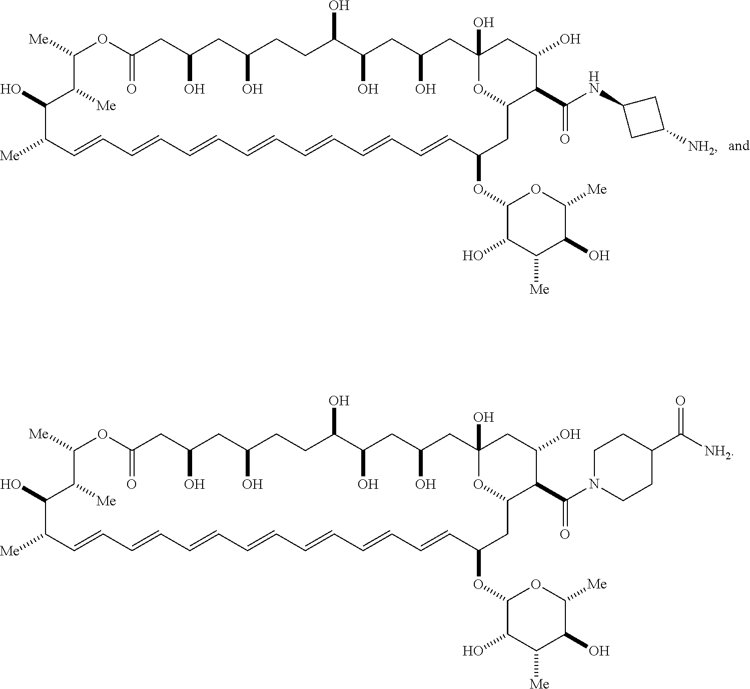

21. The composition of claim 1, wherein the compound is selected from the group consisting of:

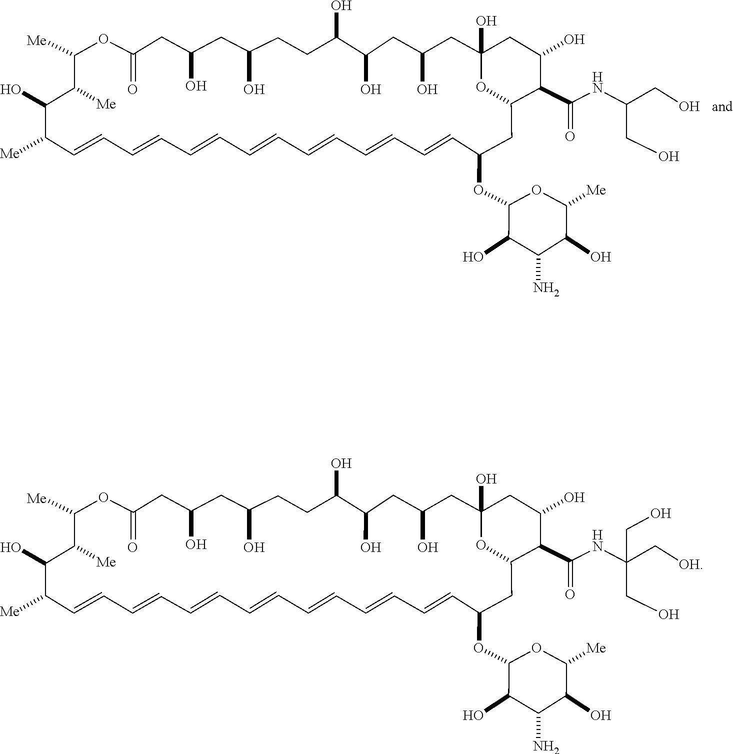

22. The composition of claim 21, wherein the compound is:

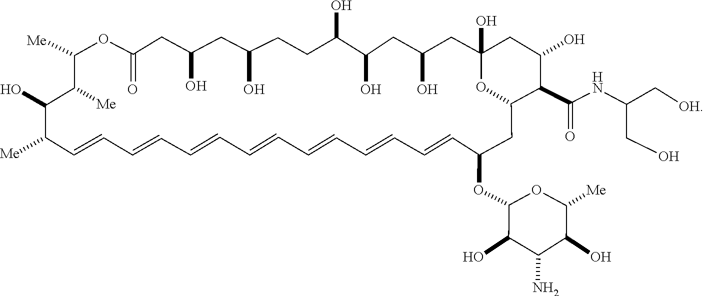

23. The composition of claim 21, wherein the compound is:

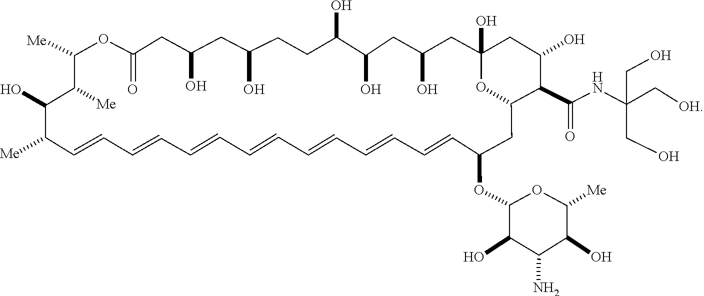

24. The composition of any one of claims 1-23, wherein the compound is in the form of a pharmaceutically acceptable salt.

25. The composition of claim 1, wherein the compound is:

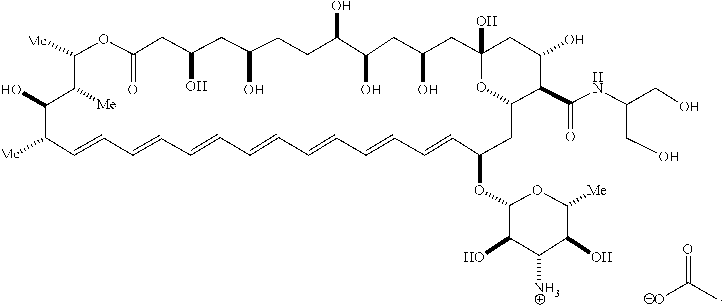

26. The composition of claim 1, wherein the compound is:

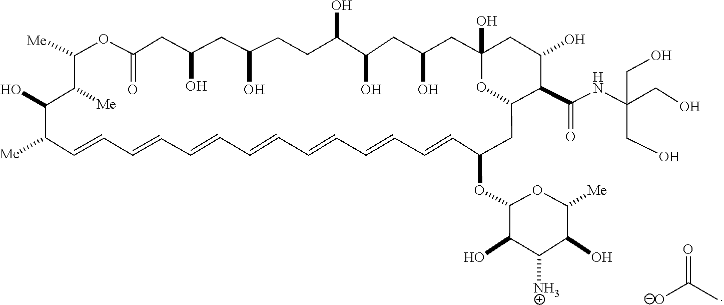

27. The composition of any one of claims 1-26, wherein each occurrence of n is independently selected from 1-9.

28. The composition of any one of claims 1-26, wherein each occurrence of n is independently selected from 2-8.

29. The composition of any one of claims 1-26, wherein each occurrence of n is independently selected from 3-7.

30. The composition of any one of claims 1-26, wherein each occurrence of n is independently selected from 4-6.

31. The composition of any one of claims 1-26, wherein each occurrence of n is 5.

32. The composition of any one of claims 1-31, wherein m is selected from 20-60.

33. The composition of any one of claims 1-31, wherein m is selected from 30-50.

34. The composition of any one of claims 1-31, wherein m is selected from 40-50.

35. The composition of any one of claims 1-31, wherein m is 44.

36. The composition of any one of claims 1-35, wherein the lipid polymer excipient forms micelles in aqueous solution.

37. The composition of any one of claims 1-36, further comprising an agent for controlling plasma osmolality.

38. The composition of any one of claims 1-37, further comprising an agent for controlling pH.

39. The composition of any one of claims 1-38, further comprising an agent for controlling oxidation.

40. The composition of any one of claims 1-39, wherein the molar ratio of the lipid polymer excipient to the compound is from about 1:1 to about 10:1.

41. The composition of any one of claims 1-39, wherein the molar ratio of the lipid polymer excipient to the compound is from about 1:1 to about 5:1.

42. The composition of any one of claims 1-39, wherein the molar ratio of the lipid polymer excipient to the compound is from about 2:1 to about 4:1.

43. The composition of any one of claims 1-39, wherein the molar ratio of the lipid polymer excipient to the compound is about 3:1.

44. The composition of claim 1, comprising, consisting essentially of, or consisting of:
(i) a lipid polymer excipient having the structure of formula (X); 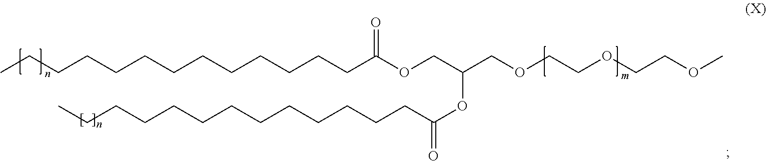
wherein n is 5; and
m is 44; and
(ii) the compound represented by: 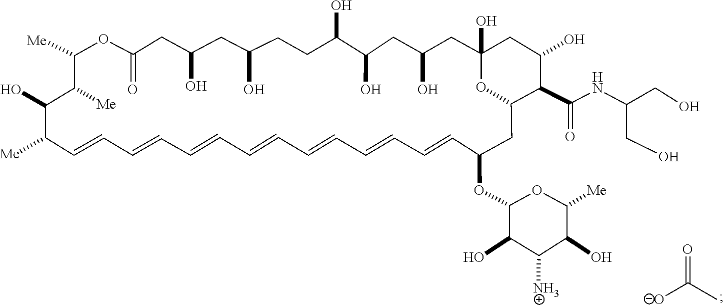
wherein the lipid polymer excipient and the compound are in a molar ratio of about 3:1.

45. The composition of any one of claims 1-44, wherein the antifungal potency of the composition is greater than the antifungal potency of the compound alone.

46. The composition of claim 45, wherein the in vitro antifungal potency of the composition is higher than the in vitro antifungal potency of the compound alone.

47. The composition of claim 45, wherein the in vivo antifungal potency of the composition is higher than the in vivo antifungal potency of the compound alone.

48. The composition of any one of claims 1-47, wherein the in vivo half-life of the composition is longer than the in vivo half-life of the compound alone.

49. The composition of any one of claims 1-48, wherein the composition is a slow-release composition.

50. The composition of any one of claims 1-49, wherein the composition is an intravenous dosage form.

51. A method of treating a fungal infection, comprising administering to a subject in need thereof a therapeutically effective amount of a composition of any one of claims 1-50, thereby treating the fungal infection.

52. The method of claim 51, wherein the composition is administered intravenously.

53. The method of claim 51 or 52, wherein the subject is a mammal; or a primate, a canine, a feline, or a bovine; or a human; or a human.

54. Use of a composition of any one of claims 1-50 in the manufacture of a medicament for treating a fungal infection.

55. The use of claim 54, wherein the medicament is an intravenous dosage form.

56. The composition of any one of claims 1-50 for use in treating a fungal infection.
